SEQUENCE LISTING (3 sequences)

SEQ ID NO: 1 - DNA, 135242 bp, Bacteriophage MikAB48
accatcatcttttagagattgtgtcaaaagattagcgatagcttgctttttgtaacattcatctagaatgtttttgttttcagagatttcatgctgacactctaccaaatcagcttcctcagttgaataagaactgttgttcttcgcggattctaactgttgttgtttagacagaatgttttgatgctgtttgtccataagaccattaaggcgctctacatcttgacgcttgtaccctagatcagaaatgactttattcagttcttgagttaatggattgataatttcttgagtttcgcgagtaagatcagcttggacttgtgacaacttatgcttagcgtctgaaatctttttatgacgatcgctctcaaatgtaccacggttcaccttagcgtcattcaattcttcagaaagtcggttctgttctttgcgatagctatcaagatcattctcgcagtgttcttcccattgagcaaactgagagtctaggcttgcgccttcttgagccttcatttctgccgctttaataccatcttccagcttctgacgcttagagttatactcatcaagatgcttttgatagtactgttcacctactacaccgccgcaatgtgagcacggttcatctttatttggctctttcaactccgaaagtttattcttcagagctactaccatatcttcgtatttttgacgcgtatcaaattgttctttgaagagtttgcgttgttcagcgattgtcttctcttctttagtgatagacatcatacattgattaagactatcctgaaggctgttaacacaaacagttaattcatctgcacttagattctcagcttcagcaatacctttgcgcgcttcttcaataagaggtttgtatttttctaacaatacctgttcttgttgagctttagcagttcttttctcttcaacgtattcctctagagaagaaacgtcagattcagctacgtttttatcatgctcgtatgctgctttgtctgactctaggctgtttatgtcttcttggatgcgattctgaagatcaactgagttagcctttttagcgctgataatagcttcaatgcctgtctggcgcgtctccaaaatggataaattttgatttgtggaagtaatattttggttcacctccttcaatcgttctttgttcaaggcgctcatctttgttattacaatgagatcgagcatctgatctccaaaacgacggcgctcttcagcccccatattggtgaacggtacaaatttgtccgcgcccaagatgatactgttagagaatagcttttcatctgcaccaagaatatcgagaagagtcttttgaaaatcaactgcttctttattcaacttttcgccatctaccgtgatttcaaatacagaaggcttttgaccgcgttcaatgatgtagtcaacaccgcccttttcaaattgcaatttgacatacagtttgttacctgagcgcgagttgataagctgtgtctttgactgccccttggcatacggtttatcaaacaaggcgtagaacaaggcatcacgccaaatggaagatttaccagatccattgtcagtagatgcaactagagtggtttttccactcttaaaatctacttttaaacccatgttgtcaatggaacgaaagttctttgcttcaagagatttgaaattgatatgagacattattgtttttccgtagtgttagccaattgcttttcgatggttaagccctttgaatcattaatttctttaaaggtctgaacatccatcttatattgatcttcgatggctttcatatcttcgttatatttcgtccaaagagccatacgctgatcattatactgtttcaaatgattgaattgctcttctgtaacgtatccgtgcacagaatccaagcgaacaatagatggattacattctaaacaacattctttgtatgttaaagaagcctttgaaagcttcacgatgtccatctgtttattacgaaaaagatgcaccatagatatagatgcgatgagcggaaatagtttcatgatattatacctttgattgctcttcgagagccgactgatataatgataacgcttcttttgctacgtcatccggattcaatttattgactatagcttcttcacgaacgtaatcatcaataacttttgctggagtaagattcattgaacgaatatcaattgcagcgtgctctataacagctagttcgcgctcatcaatgatacgtcgatcaatataaggtacgctgttaaccaacttcacaaatttgtcgtagtgcttagggtcagcgacttctttaacaaagaacttcaccactttgtttttgaatttttcaaagaagaattcatcatacgcaatagcgccgtcatattcatgcgtcacaaacagagtttcatgttcttcattgcgtatgaagttgaattcatctgtagtaacatctaaaatgaatagaccacgattaactccatcaacccagtccatccatgtcagagcgtatggcgagccaagatattctacatttccgttactagaaacggtatgataatgaccagatagaacgcgtttaaacttacggaatggtacaatagacataccgccttttgcttcgatgcctttatacatctcgaagccatcgaattcagcgtgcattagacagtaaggtgcagtagactgttctacggcttccattagtgtgtcgtgattgttcttatgtatccaacccaacaaaagaataacaggacttccgtcaaacttagaaacgccgtgactatattcttccactattttcacattagacatatgcgtaacaatgttcaaattatgaacggcgtttgtatcggtatagaatgtattgtgattacctacgatgaatacaatttcaatacccagctcagctgcatatgcacaaacttcttgaactgcattaatgacatttgtctctgtgcgcttgcggttatctaaggcatcgccagcttgaaataactgttttattcccatacctgctaacatgtctagtttacgcttcatatacttcacttggaagtctaggaagtatttgtcgccgtttttaataccgaagtggaggtctccaataaacgctacttttggactttcaatgacaatttctggaattactagcatgtcttactccacaatgcgcaatttagatttaacaggttcagagaaggtaatttcatctgaaggctttttctgattaacatcagtataaacttcgttaccaacaatcatcccaacatctggaacgctcaaaaattgaatacattctagaggtgctactacacgcgtctcagttccattgaatcgacaattgaaaatcattgattcttgatttacgtccagagaacctttagtagcttcatcgccaatagataaagcgattacatctgggtatttcttaatgatggtagctggtagaccttctacgcggtttgtgtcgcaactaatatgacaaacaatgttgttttcgtttgcccaagctacgatacctgatgcaacatgaggaaagagttttgtaaaagacatgagttattgaatctctgtttgtataattcacccttagtataactcataatagctttattttctaaaatcaaatacgagtgtatcaaaatggacaaaattagcgcctttaatgagcgagtagcaagtctgggtttgaataaagttgcaatgctctatatgggcgctgctcttgttactgagtcaactgtttcctcatttaaattttcgcaccctaataagattgctttggctaaaattgcagaaaaaattggtctaactgaaggttatactttggaaggttgcgatttggtctacgacttctctgaaaaacaattgaatgaaggtcaagaagtacctctgaacgaacgttcagactggggcgaagaaacatcagcgtgcgctactcgtttgcgtaaagcattctacgatgcgggttggcaatcaaaacctgaagacgcttacactactgtattctttgttccagaagatccagactccggtgaagttaaaatgacttactctagtctatactcattcactcttgaatacggttcagaaaaagagtcgtttaatggcgacaacccagacaagcctattgaatggatcaataaacacgtaggcgatttctacgctatcggtttaaatgaaaatttccataaacgccagaacctacctcaacttgaactcgacaagtttatcgttgatctgaaaaagtatggtatcaatgtagatgaaggtgaatacctttctgatcaactggtaccatctcagtcagaattcaatatggaaaaggtaaacagtattgtagctgaaaaaagttgggataaaaagccaattgtcatctccaatgataaagtaattgtggatggtcatcaccgttggaaagcagccttccaagtaaatggtagcgtcaaggcgttcaagattgataaatcattctctgaaatcattgacttcgttaaagacaaagactacgctgtatacaaaaagttgaatgaagcgcaataccgtcttgacgaatcatatgaagacgcagccttcattacagccaagaactgcgggcaatatggcgattgcgaagcgagtattttcaaactccacgatgaagaaggtaaatgctattacaaccttcaaattaccaattggatgggtgaattagtggacgatgatgattttgaattgttggacaccgccgtagcccatgctagagacttcggtgcccgtttcaaccctgaagatgttggttctttacgttaaatgcggattaaaaaaggggcgcaagccccttttttaacgattgtagaaatctaacaacacgcgcttcatcggatatgacatattatacatattatccaatgctcgatctagatgaaatctgtcgcgttctatatttattgatggattagatcgccccttcgtcctatagtctaccattctggacatttcttccagaaaatgtctttcatagtcgctaagaccatctagatgacggttgttgtgatattcacgagcttgtatagcacttttctgggctatgacgcgttctataggatcagatatagcatgcatgtcagtcaatttcaaaagatcatctgaaataactccacttccgtatgcgttaaaagctgatctcacctctgaagaatagccgtttgttggtgtcttcgcgatgaatgatttcatttgaggtaattcatctaggcgctttgaaaccacaccttttagcgcgcaaagttctcttttaggtaataatgattgcatgtttactccgacaaaaacagttctgatagagaaattgtaccagtaggaacagattgtttcttttcatcctgtttctggcgttctttctcttttcttttgcgttgttgttgttcatagcttccaacgcgttctcgcatatcatttgaaattttctgggttgtttcagcagaacctagatcgccattctcaatttcaccgcccatttcatcaaatgcttctacgcccatcttgttacgataatagagttgtctattctcactttctatacgatcatagaacacgcgttccatagaaatagtgatccagctgaaagcgttaggtttaccgattcttgttgatacgccgccattaaaattatgtaggtattttaaggtattctctacagcatcaccaaccatttcttccaaccaagtgtaacggttgaatttaggaaggcgcgcaaggttgacagcaattaacaatatatctcttccaacgcgttctggtattctaggcttaggaagacctgcagacattgcgtcattatattttccgacccagtctaatatgtcgtccgagagtctttcattgtcaacgtaattttgtttgctcatattgaaaacccttccacgctttcgtagtagataatatcagccttttctagatgggcgtcggaaggtttttgtttgtgcatagccttagtcgcccaaaagtctgcgatgttgtttccaacgcatgtagaatgacccttcacccacttcaattctacgtgatttttatccacaagatcaaatagttgttcaaagagttctttattcttgattaaagcagggtttttacgcttccagccgtgacgccattcgttcaccgtcttaactacatactgagaatcgctatgcacaatgagtttacactctggattagcgaattcaactattgcagccataacgcccatgatttcacaacggttgttggtaacaataccaggaacgtgttcagaataaccaatttttggcttttcatttggtttaatggctacgtatgaccaccctccaaatgaattctctttgccgttgtttgtcacaccgccgtcagtccatatttcaagaatagataacggcgtcaataattcttgaaattgttgcggatactgtaaaggtttaatgttcatcttttgcctttaattgttgaatgaaattgcgaaaatgcaaataagtgtgaaattgtaaaatggatcttttcgtttgctttgtagattgccaactaagattatctgcatctattgttaaaagagcatatccatctttatcttgatttgaaaaaagagtaacgccatcagcttcaaacttgtatttgaataacatattttcttcatcaacttctaaagcttcagcccaccctagtcgcccaaacaatagatgttttgtatcttcaaagtcttcacgaagtaatagttgtctaaaactttcagcgtcaactattcttttatggtcagaagccactacccataatattcccataggtgacatcattatcagcattatagctgtaaaaattacaatagtatccaccctaatctcctattttaatgtattagaggtttgaaataaaggattatgtctctaaatgttcaacgtttacttcttttagctttctacggataccttcgtggaatcttgaataaccagctacagaaacttcagacatacatttcaattcttcaaatttcatgattgtgaaatgttcatcaagttgttcagttgcataaacgtaaccgcgcaactcagattcatcttgagaaatataagattggttacgcttacgctcaacgtagtacaggtacgatagcacacctatgacaattaacaatcctaatataataggcatatcattgctccttgtttgaatgatacagctattttactgtaaagaccatcagatgaataaaaatcaaataataaaaatactttacttttaaaaacaaaggatttataatcagccttgcttgttgccttgtttatgttttcacctatacgcatgacaaggattcttggacgtaaaagttcagtctagttttcctcaaggtctaaacttgagagaaagtggtcagaatggtaaaattcaaatttttgaattgattcaaatggcatcttgagatttatctccttcattaagcccatttgtctaatatcaatctgatagactaccgtgtttggaagcctttacaattgaagttcagattgacccctataattgtattggatcatcagatcacgttaaacattatatgatgataaggttgagattttatcttgattctgtagtaatacagtgtgagcaacgtcagctgaacattcaggcgttgagatttccttaattgaaatctgggttcctagtatcagtcttcattcatttgaagatacacttggtcaagtgtcattcttaatttcctgaattatcaccttgtgatttatgggaagaatgtgagaaaggggtgaaaccttgtatatattagacaagagccattactaaattataggtctcacacggtagtacagctgtataaaaatagaccatttagatgctgtttattacagacgttgaaaatggaatttataaaatatctgatcatgagagaaacgtatgaaagaggttcattctttagaacaagcgatgaaatggtttttggaaaatcatagcggtaacgttatttgcgttaaaggtaaagaaaagctagaggttgatagctaccacgccgccacacaattttatcttaaacgctaggagtcttttgactcctttttatttgatttatatagagttgagtcattcggctttataatttatctgcggtgatagattgatgtttgtaaggttcattccgagattactggggttatgttcattaggggcgtgacagaaaaagtgaggtggttcaactagctagttaatgtctgatgaagacagaggttataacctttcaggtgctagtcagaacaaggtgagcaaaccataatattgtgaaaacagtttgagattgatggtagtggtctggccaatcgtctagagtaggtttcaaattggttaccgcagttgagttataactggagactttatgaaaagtacatctcaaaattggtatattaaattatgtggattatgtcatgattggtaaaagactttcaaatattggtacgcttatggcagtagctggacaaatgcaagctgttgaagcgtctccagctagaaaaattagtattccggaaactggtctggaaccgaatttcttgtctcagtctccagacggaaggactactcgacaggctgaaaagagagcagcaaaacaggctgaaaaagaccgccgacgcgcatttagacattctgtggctaagcagaacgctattacaaatggacgtaaaggacagaaacaaagtcagcagatgattctttcaactttagatgagtctgaattatgatacgcataataaacaaaagcgatatccaagtgtattctggaacgacagtttatattggacgtccaactattctggggaatccatttccgctaaatggcgaagttagtcgtaaaagagtcattgatcaatacgaagaatggcttaaaaatcaagtgaaccaaggaaatagaaaaatcataaatgatttgaaacgccttaaaggtataagcgctgaaggcgatttgaacctacaatgtttttgtagcccgaaacaatgtcacggagacgtgattaagaaagctatattatattgggaattatgatggtaagtacataattgatatctatggaagattcttatggctgcttttgttggtgtagtgaacgctagtactttgttcttggaaacggacaatgatagcctatggcaagcagcttatgatactttgagatatcaagaccccactttcgagcgcaatgcttggtcaaaatgggatggaactgtacgtatgttcaataaaaagaatggtacattcccttcaggcttgctttccatgatgctgaaactttgtaaagattacgaagacaaagttgacatcgaaccgttattgaagaatctagttagagatatcagccgagaagacattcaggaatgggttgataatctaaatgtttcatctggcggcaatcccatcaagccttacgactaccaagttgaagcgctttactacagcgtgaaatatacgagataccttgctctggcagctacatcagctggtaaatctttgatgcaatatatgaaagctagattttggctcgatatgatggctgatgaggctaagattcttattgttgttccaacccttcagttgactcaacagttatataaagacttcaaggactattctcaactcaacggctgggatacagatgcgtttgttcacgtagctggtgacggcgtgagtccatatacaagaaaaagaattgtaatttgtacgtggcagactctaaagaatcaagacagagaatttttccaaccgttcaatttcttaatgggggatgaagctcatacattcagctcagaaagcctagactatatcagttcatcttgtatcaactgttggcagcgcgtgggcttaacaggtacactcagaagaaatgatccaaaccataagatcagagtttgtaagatttttggtgattctaagcgcctagtaactacaaaacaactgcaagacagaggtttagccactaaaacagttgttaatatgatcgtaatgtcttatccaacagaacaaagacgcaaatggcgtaatgactgtactttggagaaggaaactaaaatcatatgtggatatgaaccaagaaataaaattctagtaaatctagggcgcaaactcaaaggcaactcattgtttttgtttaacaatattgagacgcacctagaagttgttaaagaagagatgatcaaagcaggtttgaagccctttgttattcacggcgaaatttcagttgaagaaagagaagaaatcaagcgctacatggaatcacatgatgacgtcattctcttagcgtcatacggaacaacgtctacaggtatctctatcaagaagctgcacaatttagtatttgcatttccgagtaagtcaaatatcagaattcagcaatcaatagggcgtatgttgcgtattcacgactctaaagacatcgcgttcatttacgacttgatagatgaaatgaagtttgacgggcgtagaaacagaacaatggaatgggctgccgagcgtttggatatatacagggaagagggacaaacaatcaaaaccattaaggttgatatttgatttttatggaatagcataaaatacattataatagcccctgacatataataactgcctaggggctttttaatgcagttgtcctttgatcatagaatggaagaacttttcaatcaggtcttcattcaagaaagcatttcatgcgagcgcaaagagaatcctcacattaatattgatgaattcaaatataaaccgtctaaagtaaaaacatcggagtttactgataatgagtaaccaagaatgggcgaagtatcgcaaattaagtgaagttgaacacgttttagcgcgtccgtccagatatattgggtctatcaaccctgtacaaagtaacaattggtatttcaaagatggaataattctggaaggaacattgacatcatcacctgcttttcataagttgttcgatgagattatttctaactcagctgaccattctcgaactccagaaggaaagaatcttacagaaattgacgtacaggttcaaccagaaacaggtctcattattgtaactgataacggcggcattccagttgttcttcatccagagtacggtcaatatattcctgaaatgatctttggtgaactacgttctggttccaactttgatgacaacgaagactctttatctacaggtcagaatggcgaaggcgcttctttagtaaacatcttcagcgaaatgttcaaagtacaaactatccataacggtaaaatgtttgaacaggtattcaaaaatcatatgacggagcgttctactcctgaaatttgtgatattgtttctgaaaacggcacaaccattacatacaagccagacttgaagattctcaacgcttactttgatgaagacaccttactgcttctagagcgccgttgttatgaaattgctttcaccaatccacatttgaaggtttggttcaacactaaattgatcaacataaacaattttgaaacgtttgttaattcatttgataaatccactgtagatatttctcaagaccgttggaatgtagcagtagggctttcaaagactggtttccaacatatctcttacgtgaactcaacagcttcatttgacggcggaacgcatgttgattatgtcagtgaaaagattgttgaagcagtgcgatctcatattgagaaaaagacaaagcagcagatccgaccatctgacatcaaaaacaatttgcgcttgttcattgattgtaaaatcaataacccacgctacaattctcaaactaaagaaaaattgattacagcgcctaaccagtttggtagtcagttcaaaatttctgataagcagctgaaggctcttctagcgtctccaatgattgaatctatcattgaatgggctaaacgcaagaaggagctggaagatgcaagatccattgaagaacaagcgaaagctgtacgtaaaaacggattctacgatattccaaaatatcgcccagcaacatctaaacaacgcgaagactgtattctattcctaacggaaggtgattcggcattgaagcctttgggtgctgcttcgaatccaaaggtacatggtctatttccactgcgcggaaagcctgaaaactgtttcgctaaagaagtgaaggctgttttgagtcaagagattctttatgttatgagtatcttgaatctcaagctggggcaagatgaattcagtgatatgcgttatcaaaaggttgttattgcgacagactttgacttggatgggatgcatattcgcggtctattggtattgtttttcaaaaagttcttcccagagctattgaaagcaggtcgcgttcaattcttcaatacgcctaaccgcgttgtcacgtacaagaacaaagagtacgatttctatgacgatgaaacgtatttcaaattcattgaagacaaaccatcagccaaaactaaatactgtaaaggtctaggttcacatagcagcgagaaaattcaatcattcttggtagacccagcttgttttgtgacagtaaactatgaagaagatcgtgatgatggtttattgaatcttgtctttgaccctactatggcagacgatcgtaagacttgggttggtgtagcatgatacgttatgaagatgaagatacaaatattagtcaagaaaatcaaagtgctatgtttgttcctagaataggcgaaaacgtgatcatgcagtctagactgtatgtcgtaataagggtaattcatacaacgcaaaataatctagtgcgcgtgatattgaaagaacgctacagagtgtaaatgatagtataataggggcttataaagccccttatttttggaaaagcaaaatgagtttattgagttgttacacagatactgagaccactggtctacacccttctaagggcgaccgcattgtagaggtttgttgtatcttgactgtagacttcgagattgtagatcaattccacactttggtaaatccagagagacctgtaggattctcggctactattcacggtcacacagatgataaattgatagacgccccattattctctgatatatcaagagatttgaagcaattcataaagcaagctgataatcttattgcttacaacgcgccttttgatatgaaatttctacaacacgaattcaattctaaagggtttgggccatttaatatcaaaaatcaaatagatgtgttaggaattgctaggtctgtatttcctggcgaaaagaacaaattactaaacgttattgagcgtcttggaatagactatagtaatgaacagttgcatacagctttgtctgacacaaagttactcattcaggttcataaagaacttttatcactacttaataaaaacagcggtcttatttgagagaataccaatgcttgtaatcactattgatgaaaagaaaaaccatgtacatcgtaattttgaaaaacctagtgatgtcaagaagtttcttcgtactcgaaaattatctatcaaacccaaaggcgtgcaggtagtgaaaggcgtatattacaaagcctataccgttcattcttatttgaaagatacgacgcctagatttcacacattattgatgaacatttgaaaagaattaattttaggaataatcctttacttgttcgcaatattatattatacttggtctcagataaatcagtttatctgagattttactatcaactcaacggagttacaaaatgtataccattgattctttttctaaagttcttgtgagcaaaaccaatccagctgaaattctcttggctgttgaattcaatccaaattctcttcagtattcgcaaactgtttttcgtgatgtaatcaaagctacagattcaacagttagctacgcgttcaatcaaataccacttccagaattcaatccagaggtagatgctattgtacgtggacgttctggaagcgtattgatgcgcgcagggaaagacctgttagcaatgaaaattggttctgcagctagtcttgtgatagataaagtttatgctaaagaacatttctatgaagttgatttgagcaaaggctttaattatatccacgacaacattttatacactatcaaagaaggtgaaagcggacttcaagcgctcatgtggcgcgcgccgtacgggaaaactcctggttggacttcatcttacattccattgcataatactttcactaagaaaggcgctaaagccgtaggtgtgcgctatttcaaatcatctgatatcattatggattttggatctgaagcagctgatctgaagataacaatcaattcggaattcaacgttcttttggaaaattttgaaacttgtgcaagggctgatgaattcaaaggtagcctagatccagaagacgtttctgaagtagaaaataatctcaatgtcgcaaaaactaatcttgaagagttctttgaaaactccgtggacgaacgccttaatgaattctttgaaaaggcgaaatcagaatacaaactgtctttgaatgtaaagaagtgttagtaaaattttaggagaaacaaaatggctgacttttgtgcatgctgtacttttagactcggtgtcgatccaattttaaatgatttgaatcgggaaccgaagatggatgactcagagattgatttcaatttcttggtctataatgatctttgtgaaggctgtggacatgtagttactgtcgatcaaaaggggtatccagcttcgttgccgtattactatatatacgatggccaagatgttgtacatattggtaaaaataaaccgtacacgatcattgctcttgcaaaccgcactactgcaaattaccctcatatggttgtttatcaagatgacatgggtgcggtgtatactcgatggctaccagtgttcatccgtaagttcaagcctaaaaatggcaattggttcaataaacctgcatcggaattgaacgcggaaatgtttctagattaaggttataatttagtttttggagtgaagagacatggatattgaaaatcacgattacgaaaattgcccaaagtgttggttgtgtcatcattggaaatcaatgattggcgatccaaaaactttagttgacgaaaactacggttgttattctgacggtggttgtaaacccaatgagacaaaatatctaggtcacgctgggcgcgttttcatcattgaaaaattagatggaaagcgcgtagtgacaaataatttatggagcgttgggacagttcctgagtatttcagaacgcctgagcgcttaccaatcaactgcaccataaagttgggtgaaagaaaggatttgttcgaaaatgattaatgtaatgtgcctatgtttgagccttcaatatcctcatccagagtatattggaagtgttcatgttaattcaactcactacattggagtggattcgagtgtagagttgaacaaaaagatgatagacaaaatcgcatcatctatggttagtgaccgacgcaagcatctagaccgttgcgatgataaaacagttgtgacatttttcgttacctctatggccaaggttacagaggtagagaatttttctgattttgcagatacaaagcgtctgctgttggtagacaatatgtctctacccttcatacacgcacaagatcaggaagaaacgtaatgagactgaaacttattcttgtgatagcttgtaagattaatcaagaagtggaagttgagaagggtatctgtcgcatcgaacctgttgaccgttcattcactttcttcataaaatcggaacaaggtttaatgactaagggtctcattgattcttatactcaatttgcgatggagcgtctgaaggaactttattcagctaaaaatttgcgaatgactagtatttctgagacgagtctatatctagctggaattgaattcgtgaaagaacagggtttcagctactcatttacttcatatgatcaggagatgtagaatggctacatggagttctttaaagggcagccccattattcagcaaatttcagacagtatcactaatgctgatgagcgtttggttgaaggtatacgcccctatcgtagaaagtctattcaaacttttgaaggtggaaaggatcaatattacgccagacacattacacgcgtacattctttctgaaggcactaaggaatgggatgttcctcagtgtataattgacgatttttacgacttcaaccgcgctgaacagacgtatgatcgggaaagcatagaacgcttgctaataaaggcaattatcgtggaatatgagttgaatttccatttcagaactgaggtatggacgtaacgcaatcttccttagcgccaattgagacgtctaaagatgtctcaatcagaaattgataaatacaaaatttgtatcttacagactaaaatagaatatgatctgaaagacatgttcaattgtaaaatttcccggaaaactagatatgaacaccaaacttcaacaagtagatgattttatcaatacagattttttaccttacgccgtctacgactgccagcgcagtattccgtctatggttgacggtatgaaggtttctcaacgtaagatcattcatacagttctgaagactttgaaagaaggcgttgacatcaaagttacaaacttgggttcagctacatctaatgaaactcattataaacacggtgaggattcgattgtagatactgtagttggtcttgctcaagattttgctggatcaaacaactatcctctactggaaaaagaagggcagtttggtacagccattgataatgatgccgctagtccgcgttatattcacgttcgccgttctaaattgttatctcaaatgtttgaccaagatgacttaaacttagtggaatatcaattgtttgatgatgaactgattgagcctaaattcttcctacctaaaatgcctctaattgttattaatggtggttttggtattggtacaggttactcatcaaacattcttccgcgctgtcctaaaaaggttaaaaagtatatggaacaggtggccacgggtgcaattattgaccaaagtctactaattcctcattttaacggcttcaaaggaactgttgagcgcctagaaaagaacaagttcatgatcaaaggacttttggaacgagctggacaaactacaactatcatcaaagaccttccgccattttcttcattccagtttgagaaatacaaaacgcgtgtgctattaccccttttagacgccaagaaaattactggttttgataacgaatcaactgaaggtaattggcatattgtaattcatcactctagagattttggaaaacaaacgcctgaaaagattgccaacattctcaaaatgaatcaatcattcacagaaaatattacagtttggggatacgataatcgtatgaaaaactttggtagtattgaagaattgatgaacgtttggatgaatgagcgcttcaaatatctacaagatcgaaagacgcatatgcttcaaaaaatggaagacaatctgaagtggtatactgctcttatggatatgatcaaatattggttagccaaccctgatattttgaagaaaaagcgcccagaaattgaagatgaactatctagcgtatctagcaataaggcgtacatcaagaagtttctagagtctgatattttatctctaacgcaagaacgcatagataaatcgcaatcaattctagacaagattgtatctgagcaaaaggaattacattccaaaactccagttgacattatgatgtctgatctcatgagcttaacaatctgataaataatatcagatgtggtctatagaaagggggctaaacagccctctttttatgtgccttgaataagtagatacatggggagtgattccctacaataatgggggataacctgtgtttctagtagatcggttgcttgaagctttaatcgctggcggtattggcatcttcgggggtgtctctgcatacgtctatcggcttaccaagacaagagaaaagttcaacgtttggtctttcgttgcggcagtaataattgctttctttgtaggaaatgtattcggtactttcgtgccggaagacctaacttatagagatggtttgtttatgctttgtggtttcctttcatacccaatactaggtttgttggaaaggggtggagtagattggttgaagtcttttattccaaacggttcacaattgaaagacaaacgggagaacaatgatgagtaaaacatttcaaagtcgtcactccggatctgatatcgaaaagatactcagctccgttggcgaaaagatatcatatgatgatattgtagacgattatcaaggcggaggtaatcaaaaggttgcctccgcagaaagtgtatccagactttataaatggtctaaacagtttgatgatcaagtgtggttgactgagttattcaagtctatagaaggatttgacgtattcacagaagatgatagacaacttctcaaccgcctaaaacgcggttttgttggtacatatcaagacgctattgagcgtcagtctaatttgtcgacaattgatctttcaggtggtgaaatatctctgttgaaaaacaagtctggcgttcagtctcttgaatactacgatattgttaaaaagaattgggtacagtgtaaatggactccagaagctactcaagctaaacaatctgaagtatctggtggcgataaggttgtatatcaatttgaccgttacaatatccaaactgtaaaactacttgttcggtctgagacagctaacgatatctgcgccgtagaagttctgatgtgcattaagaacggtaatacatattggacaacttctggatatgtgggttccaactcagaactcataaaggtgaagcgcatgtatttggaaggtgacgttgctaagttggagatgacactagcttcaggttcaactacaactgttcacaaattggcggagttctaatatggatgtatcagtagcaggtttgaagaatcttatctttaaactacatcgatttgtgaacggttcagcagatgaaaaggttcagcttgaagatggtcgagagatcaaaaccatggctggtatagaaaaggttggcgctgaacgttcattagtgcaacaggttagagattatgacaactatcaactggctagtgtagatgctctaggtggtgtcatactacaacaaggcgacattgtacgtattcataatgacccagacaaacgtttgcgcggtttatttgaagtcacctcagttaagaatcttagacgcgttgacggttatgaactgtttagctttttcccagtggaaggaactatcaatagaacgtcaacggatgagcgtcatgtggcatttgaggtcaatattgataaacctttattggggcaaagtactcacggcgctttcacatacgcagcattcttacgtacaaatactaatgtaaaaacgctattaaacgggactgtagtgtacggtattatggaagatggtacgccgtatgttgaaatcgacaaattaagacctaatgtgtcattggattttatccatcaggttgaagatatgggtgaatatttctcgtatcaatttattgcaacgcctttggttgcagtttctaattggacgatctccactaaagttgacggaatataccaacctgaaaagtattgatccagcctaccgtgtggcgttggatttttacatacaaaagcccttgtctattacaggggctttttattggattttagaaaataatgttcatgactatttgtttatttgatattgcgtttttacaatatataatagtataatattcgtaatcataacgaccgaaggtaggaaaataccaatgtctcaacaattgaccccagccgaacgcgttcaagaacgtactgatatgcttgaacagatccataatgaggtagatgccttattggaaatcaactctgaacaacttgatggtgaagcgttgaaacatgtcaatatcttctctaagttacaacgcctttatagccgtgaattcaagaagttagacaaactgactatctctcatgacatgctgaaaggtcgtttgaaaagctactatggcggtaaagcttcagaagagacatacaaacaacgtccattacgcgtagctccactgaagtcagaaattgatgaatacatcaaagttgatgaagagttcatcctatctaaattggaacttcagattcaagagcgcattgttaaatttcttgaagaatccaagaaagctatgtctgaacgcacgtacaccatcaagtacgctattgacttcagacgcatgatgaatggtaacggctaatgaacggcgctttatctattacattattggttgcaaccattatagtacttctcattattgagctgagaaagcaattggggcgcacatctaatatttcagcttggattcagaccgccgtttgtttgatggtagcctttatcattggaagactttccttatgaaagacaaactaagaataactcaatccagatatacacgcgtctggattacaggtttgaaagagctttcagaatccgaaaagacactacttgtggaaaagataaaaggcatgggcgcttttgtagagaagcgttttaacttttctattccgctcgctgaacgcctcaaagacattaaatttgacggtgagcatgtagtcatctattatgatgaaaaggttgggtggagagccgttgtttctaaattccaaagaaattacgaagacgttggtggtacatgcttttctgccaagaattttctgagtgaggagtgtgacactatagttacactaggggagtaatgtggtttcatttgcagatttaaagaagataacaaccgaaggtgtaccaaagtctggaaataaggtatttccaaagcgcatcattgagagtttagtgccggagtcaaaatggggcgagacttttcattttgatggaagacgttattatagaacgcctaatggcggcatctatccgtcagcaactacgtttctgggggcttttgaagacgaaactcatctcattgcgtggcggaagcgcgtaggagaagctgaagccaatcgtatctcaaaacgttccacagatcgtggtaccgctattcatgacgccttagagcattatgtacagggtataccatacgacgctgaagctgcaacaggcgaattctttttcatgtacgagcaactacgcgttgttctaggtgaattggatgactggtcgtatgttgaacatgcattgtggtcagacaagttgcgagttgctgggcgttgcgacttgatgggtacattccgcaaacgtagagctattcttgactacaagaacaaaaactatgttcgaggtcgagaagacatgcgagactacttccttcaaggtgctttatatgcagtaatgcatgaagagcgttatggagaaaagattgatgatatcgtcattctagcttcagaggaagataagaagcgtaataaagcccacgtattcattgaacaagttgataatatgagagatgagttaatggacaagtcagctcgtttccaacgtcaatttgctcattggtacgatattccacctttgatgttattgtgagattgaaatgataataggtgctgcagtaaaggtcggggacatattaatagctgttcccaacccactaagacattctgatgcgataagaatgatggaacgactgaacttagacgagagcgtggctttcgactggggcaagggactgaatcaaggttttgttgatgagtacggaaagtactacaacagaactgaagctgccgcacacgcctttgaatgtggacaattgaaggttgaattatcggcggtctgtagcgaagacctttggtaaaaaagatggataataggggctgcaccctattatccattggagaaagtgactctcgtcgtaccgtcctcgttgaaccattgctctactgtcatatcagaaaagtctagtgtcaaacccattacaacatctgttgtttcgccttcagcatctagtgataaaggcgcaaggttgactgcatgacagtctgagaagacaaaggcaacacccacgggcttcttattactatttagcggcagtaccgcaaccgattttgaatgagcaaatggatctggattaccagtacaatctttcatccacttgtaacaatcaatatagtttttccaaagttcatctacaaagaagttgatttgaagttggtctacctcaatgtgagatcctggaatccttatttgacggtctgtgtcggtgggcacaattgtagatgaagcgctgaacataggcatctgaatgtcatacaatttgaattgaatattctccatcatctcaattacaaccaagaaattgtttgactgggcgttggaatagataatatcctgcattttgtttgacctcatttgattattaatacttaacagcttttggggttatgattaacccgatatcataggagaagagatatatgtctcgtttttacacttcactaaccaagcataaagaaaaaatcattgaacgctacgtagaaaatggcgttcgtggtacttcagaaattcatttcaagcccaagttatttgtaccatctcaagatcaagaatctgagcaccgttctctaggtggtctacccttacaggctgtgtcttttgataacatctatgccatggatcaatggttagatgaaatgaagaacgttcctaactacaaaatctacggcatcgacaaccctctggctcaatatgtggctgaacgttatcctgaaaagcagattacgtttgatatgaaacatatccgtggcgcgcttctggatattgaggttgagtctggctataaagacgcaaaaggcgttatctttagtggcccattccctgaacctgctctagctcaatacgaagttacagccatcacggtttatgacacatttacaaataaattctttgtatttggtcttgagacgttcaagggtgaatatattggtactttcgatgtagaaaagttaccgcctgaagtagctaaacatatcacgccagaacaaattgaatatgttggctatgactctgagctagacttgcttgcagattttgttgatcatttcgcagacgctgaatacaactatatctcggggtggaacagcgacacgtttgatattgcatatttctgtaaccgtattgacgcccttcttggacgagatcagctcaaacgtatttctccactagattacgtgcgcaagcgtaccttccaaggaccttatggcgaagaagtgactttcaacattgcaggtattagcaaccttgatttgaaagacatcgttgacaaacacgcctttgtggaattggataacaaaaagctaaacacagccgcaaaacacttcttgggcgaagaaaaggttgaccatagtgattataaaaaccttactgaattctattacaaagactttcagaactacatctcatacaacattcatgacgtcaacttagttaagcgtattgcagaaaagactaagatgttacaactggcgtacacgctggctttcttattccattgtaatccagacgacacaaatgcgacagtacttccttggcaatacttgatgtacggctatggagcacgcggcaaagaatattctgaaattcgtgcgcgcgtgaattctaagcctgattacgctggtggatacgttaaagatttgaagccaggacggtacgaagacgtggtttcagtggatgctaatgcccttaactgataaggggcttgtattagtaataatacaatgaaaatctctttaattgctgggaactctcactgagacaatcagcagccaagtcagattgaagtctgaaaggttcaacgactagacagtcgcaagacatagtacagccaagtgtggtgcctaaatgtatggcttaattggaaacgggagacacggtgagtatagctcatctgcgctagttacaacagcgtaccaaacgtataacgtgggtgatgatatagtctgctcttgtatgcgaatacaagctggatgtaattccgggatgagaatcacgaactcatctgaacataaaggatatccacatatggcgcaacagttcaatatgggcgttgaaaccttaattccttgggccattacggaacagattctagatgctctggcaaaagaattggaatctttacctgaagaacaacaagacaaatatacgcgtttgtatgctgaacgcttccgcgctcgtcaaggtatttgggatatttactttgaacgcccttacgacttccaagttttaaagcgcctcaaagtttgtatggcaccaaacttagctatgttcaagattgagcgcccttcattgttgcgccgtgtattcaaagacttgtacgacacgcgttctgctattaaacgccaaatgaagaatgatgaacgctctttagaatctatcaagaaaaaatatcttgatgaatgtctagagaaggacgcagggttctataagcagaaggatgaatgggaaacaatcattcagaacggcgattcaaaccaacactcgcataaaattgctgccaacgctggttacggcgcgattgcgaatgaaaactttgctgaccactttgatattcgtattgcagaagcaattactacagctggacaatgttcaacgcgtcatatcatgttccgcttgaatgaatggatcaataacaaatacggtctaacaggtgatcactgtttctacggtgatacagactctgtgtacgttgaatttaaagaagtggtgaaagccgaaggttgggataagttgtctgttgaagctaagactgacaaacttgatgaactcgctaaggacgttcttgaaccactattcatccaatgggctgaagaactagctgcatcatataactgcccgatcaatgctcttttctttaagcgagaagcgatttctacagttgccatctttactactaagaaaagatacgctatgctattaacagataatgaaggcgttcgattcgcgtctcctaagctgaagattgttggcttagaagcgcgtaaatcaaactacccaatgttctgtcgagattggatgaagaagtgttatgaatttgcgttgaatgatcaaatgactgagatgcatcagttcgttgacgcttgtgaaacagacttcaaaaaggatcgagatattaatgagtacggcgctgctcaaaatatcaacaacattgaagattatctagatagtagcggtaattacgtaaagggtacaccttatcacgttaaaggcgcggtgaaccataacaatctattgaaacggttggaagtgacacatatcatgccacttcgtagcgggaataaatattttgtgttaccattacggaagggttcacctttcggtatggaagtaatatcatatgatgatcaattgccgaaggagttcaaacttgatcagtttattgataaagaaacagcattcaataagtgcttcatttcgcctttgactaactttttaacagcaataaattgggaggcaaaggctccgacatcgttcagccttgactccctattctaggagtagagaatcatgatgctaacgattcctgaaatttcaaaacaattccattgggttgaaccagtacaagatgaagatgttgtcttggtaaatgctcattcattgtgtttggatttacagacgtcgtttaagaacagtatttctacggttgaagaagtagcttcagccgtgtttaagttctttgacgatattgatagtcgtgctgtgatacgcgccttgcaagacggcgtttttgattttggaaaggagcaaaatcgtcttttaatcaatctattcgcaacatggattaaaaagagcttttacgacattcgtgaattcagccgttttgtagtttcaaaagaaatgcttggaacgttgattgaactgaaacaaaatgaaactaagattgagctcatgcgcaagggctttttcgtagcagtagttttggctctagtagtagtatttggactaacgtatctaggttattcagttgatcattacgccatcattgccttatccattgccattgcatcagtaatggggtttgtttggaataaacttctgaagaaacaagatgaactgacggcaatacttttgtcttgaagtaatacaagaccaaccaaaaaggaagcgtaatgcttcctttttgatttttataataatgtaaaaattaggctatgattaacccaagtatatggagttaatagttatgccaaagtattctaaaacgttcaaacaagatgtattcaaagcaatacgtcagggcttacatgtaaatacaattgcttcaaaattcaacataccgccgtcaacagcaagactttgggttaaagaatgtgcagctgctcatgctaaaggtgaaaaggtagagacagtcaacctagattctgcgcttccatcaacgacagcaatcgtaagtcgtaacatgaataaggttgaacgcgcatttgtttctaagttgttagagacatataagccaataaatctatctccagaatcatctgaaggtggttctgtatcagcttcagaaaataaagccacgcatcatatcatttttaaagacttcaacgcattctatgatcgcaacaacgataacctagatcaagcgttgagcatcagtcttcaaaaggctcaggctataatcatgtctaaaggcgaggttgacgtgaaattacatttcattggagaccatttcaacactggaagtctttatatgaagcgtattgatgaacctgaacaataccgcgtgtttatggctgtattccatcatatgcacaatctcatctctcgcgtgaaaagtgaattgccatttcgcaacgttgatttccaagtagaaggcgctgatttccgtggctctttgcttgatagcttccttcgtcagacgtttatgatacctttcaccaaaccagaagataatatcctgtttttgtctggtctttcttctaaaagcaaagaagatgaacatatcaaacgttctttcagaccatctagtggcgacattgtacgcactgtatataccgatcttagagatatcagccttgggccaattgagttctcattcggtgacctcaatatgctaaagggttgtgcatctatcaaaacccttaaaggcaaacaactttgtacagtaaatattgttcattggtaatatttggagaccataatgactgctttaattctagattctaaacatcagaaaactcaacagccccaaactgaagagattgacttgcgcactggttcagaacagaccttattattcagtgaacaggcagcgcgtctacttgcctcaatcaaacaacaatatagcgaagcgaacgctctcattactgaagaagatgctcaagcgctggaatatcacattaaccaacagattaaaggctataaaaacctagagctgtacggcggtctgaaaattgtttctgctaaaattgaattggttcaaggtggcgtagacctcaaaatgacgcttggtgtagctgaagacatgcgcttatacaaaaattcaggcgctgaaaatgaattctacattggttggatggaaggcaaagacctatacgtcaatggcggtaaattgcgcattcgctcaagtaataatcttggcaatgacaaaattgttactatggacgtttctcaattgcctgaagcgcgtgaagcgcgtgagcgttctaaatggttgtataaagcatgatcgaacttttttctatactattggcagtccacttcctttgcgactttccgttgcaaggggattttgtcgctgttcataaatctagacacgttgaccctaactggccaattattcttttagctcattgttttatacacggtttgggcgtttatatcgtactgaaggcgcttgggcacgagtctaacgtcgctatggtctcaatgtggtttatggttttatcgcatttcattattgacttcatgaagaatgacagccgtttaacgttttttcaggatcaaatggctcatctaattattgttataatcataacacttggagtgatgctataatggatattggcgttgttgcttttcttggagccatttgttgcgttctttgtatactatatgaattatcaagacgtgaaaacaaacatctacagaaagttctagaggactcgaagagcaaatacaattatctcttaactgagcataataaaactaaagaccgcatggtcgaaattgaagttaataatatagagctacggcgacggctctatgatgaaaaaggtgtacatttaggtatgcctgagtaatttatcaaaatgagacgcaagtctcatttttatcggagagtgaaatgagtagaacgcgtaaaggcagtaaaccagttggttacgactattggacagctagaccttgtaacaaaggtggaggttgtgtaggaaagtatacaaaaattatgacgcatagaattgagcgtcaacaaggaaagaagcaatccagagaaatagacagccaataaaaaagccccgcaaggggcttttttgtacctgagaaaaattagccttttaatttttctttggttgaatcagcaacgctgaatgttacgcgtttagtagcagcaatatcaatttcttcaccagtttgcgggttgcgacctttacgcgcttcaactggagttactttgaatgaaccaaagccgataagttgtacgccgttaccagctacgaattggttagtgatttctgcttcaattgctttcactgttgcttcaacagatacttgagtttggccagttgtttttgctacagcagctacaagttcagtacggttcatgtctattctcctaaagtgtttgagacattaatacaggttattacctgaatgaagaacattatactgtattatcagcaccaagtaaaggatttgatttattattttaattggaatcctcacttataatagactcatctatgataaaaccaatgatgggattacagttatgagcggaaatgcttttaaacaaacagaacgtatgagtacagagacgttggaaaacttaaagcgacttttaactactctttcagcaggtaatgactttaatatatcaaagcctcattttcctgagttctttcaatctaaagaagatcacggagaccttgacgtatttcttgctgatactgttcaaaaccgttgtgcagtcaatgaattggctatgatagcctatcgtaacgagcaacctgatcatccttgggttgatatgggagtattcgctaaacgcaacagtacagtagagccgtcagaacgttacttgatacaaacggaaggtagttctcagctacggttgaaatcaaccgagggtaagtggtgtcatgtagatgttcagtttgtacctagcaactgcttatctttcagcgcacattattattcgtatggcgacctttctcagttattacagatatggttaagaaacttcaagatggttctcaaaaatacaggactatactataagactgttaccgcaggtgtggagcatctagtattgataactacagactggttcgcggttttagacatcttagaattgaatcattcgccttacagaggttacgcattcgaggattttcgagacgcatttatttggatggaaaggtcgcatctgttcaaacctgatatgttggatttaagagaaggcgataaacgcgcttctagacctatgttcactgctctgcgagaacataagttcaatgaatacacatatcagcgtttatccactatagatatttcttataaggtattggaacggctaccagaagccttcctagccatcatgatggctaaggttaatgacctcgcgcattgctcagtagaccaacaattttacgctttgaacaacatcataaacatctcgtccatgaagcgtagcaatttcgatggtaataccaacctcagtgtatttgatgacggcggttatagcaagctagtgaaggataacaaacttcctttctagacaaaagaaaagccccgataagggcttttctttatttacaagataattagaacgctttattcaatatagcaatgattcttgcatcttcagcgcctagattgcctacaagagaagctagatcaccgaaacgacggtcataacgttcacggtcagctaacacagacatgacatagatgtcattttcttcatcaatattaacagatactgttttgaaagtacctttgtttgagactgaagaagtgatcatgcgatgggttttagatacctgagtgaatacaacatctgaaaagcggtaattggcttctagatcatcagtacattctttcatcacttgtacaatctctccgcgcagaatgccgccaattgttttactgtctagtttatgaaaatcagattctactaagattgtgaaattagttgtcatattgactactccattgagtgttattaaacaggtttgaaattcaaatctgatatctattataatcaatttcttgggcgaagtaaagtgattcatttgattttttaaaaatatttcgtttgctattataatagatcattgatataataacgagagcaatctcatgttgttaaaaccatttgttgaaagtccagaacgccgacaaggtatgcggagaacgctgatgatgtatcatttaacgcctttagagaatctagactcaatacatcagttcggtctactcgctaaaccaccaacacaaacgtctgaccagtatgatccactaggcgtttatctttcttctgactgggttgaacttttggcttctggtacctatccagaaatatttgattataaatctatagccgttctagaggttgatattcctatagaacacgaagcccttgaatgggatccagagtatccgtctcattctatgattcctgagtacgaattagattttgaagtcagagtctgtcccaagaatataccatctagcatgttatctttatttggtgcagtacatcttgatgacttagggttgggtgagcaaacctttcattcgtcaatgactcaaaggatgaaacatattcgagagtgcctacatagtatgtttccacactaccgtctggaaggtaaaaattacaccactagacaattggaaaatacagactcgatttttgtattttaacgttttttacttcatgaatataggtttaaagatatgacatttgaaatacccaacgttggtttcaatcgaactcatcgtttaaagtttcaatacgatgaaataacaacttcagaatatgtgcgcatgtttatgcacttactaggggttgaattttatactggaaacagttccgtagatggttacatacgtttcatagagacaggaagccgtgatgcgttgtatattcgaggtgatatgggttatgctagaaatctatcagaaattgaattaattgatgaagacggaagtcgtatatcatataaagaatataccctgaaatattatccagaaatgcgtcatattattgatgagcagatcaagcagtacaaattgtaggaaataaagaatatgtacggaatacctagaacaagccttgatggcgtcttaatgacactagatgattcattgaattgtaaaattggcatagctcgtcttaacacagacgattctatggaattgagagacgttctaaacggaaaacaagcatgcgtagcttttcagtgcgatgtatcttacaacgagttctacacctctcataagataattacattcgttcagaacggttactgtattttagaattacaagaaggcgaagccagattactttcccgcaatctttcgaatccaaagtattacgttgaaggcgaagaaggaacatgggcaagatgtgaaatgtttttggacaaagatttcatagagagccttaacaataaggatgtagaagtaatcttggagtcatttcaaaaaacaattacgccgtttaattcgcgccgtcgtcaagtgatcgataaaataattgaatcatcatgtggagaactttaatgaaagagatcaatgaagaatgggcggaagtattatgaatattgatcttgaaaatactgtacaagtcaaaataagcaaattaatgtctgaatcattcagcgtagaaaatattgaagactgtcaaggtgaagttcacagcaatttcgtatttctatcatacactaagaaaggaatacagttcattttatctcttagaaaacttcaaaatggaacgttggcgctgaaggtttgtgagataggaacttccgttttagaaatagagtctatcctacaggttgaatgcgaccttgagagacacctgactgatcataatttcgcaaaattattttttcttgaaaatataaattagtcctttacttcttgatggtattgatacataattctactcataccacactaccgctcaaaggagctacataatgaacaattcagttaaattccaccattcagcttatgattcttatattgaattctctaatgaacaaaaacaaccacgcgctggtttgtttcttgatcgtagccgttataacattgatattaatgaagctatggctaaggcaatttctggtgttatcaactacgaaaatcttgaagttgtagacatctgtgtcaattgctcatcaatctctttcagtcacattacagaccacggcgtggttatggttcaggttgtacgtaatagcctagagtcatttacagtaacaacaataagagatggtttcatgacttcatcagaggttgaaacaatggaagggttttataatgacccagaagattttgaagtattgatcatggaagtttgtaaagactattggatcaaatggtagaaaagaaaaagccccgcaaggggctttactttgttcataatccacattataatagctgataccttgattatcatctcaacggagatacaaatgttatattaccacgtcacgcctttgaaaaatcttgaatccatattatcaaacggcttacaaattggttcagagccaacgtctttggaatcagaccttctaggcatctatctatcatcttgttggaaagaacttttgatgagcgatacatatccagagataaatcaccatcttgagattgctattttggaagtggattgttctagtctagaattaattgatgacccagaatatactcaaggtatgtttgaaagagatgaaattgattttgtagtgaaaatacatcaatctaatattccaactgagcgcattaaaccaattggagttttgaatttagataaacaaccaagatacttctcagtcaaattgataaatgaatctttcaaactattataaaaagaaaaagccccgcaaggggctttttctatacttgatttttatccattgattggggtttcaactattattaacgtagatttaatatcggagtcaaacgtgttttatgtttatcagttgaagataattggcatcgatttaccattctatattggaaagggtaaagaggaacgccataaagagcattttgttgggtatatgatcgaaaccaacaaaggtaacaagcataaaattaataccataaagaaagcgcaaagagaaggtttagaagtcgtctccgaaatcattctggacaatctcacagaagccgaagctcatgctgctgaaatagagtatattgccagatacggaagaagggatttgggtacaggttgtttaactaatctaactgatggtggagagggtatgagcggatttgtaccttctgaagatcaacgtcagagacagagtgaattactgacacaacctgaatacagagacatgttccaaaagcagactgctgaattatggaaagacgaaacgtatcgcaagaatgtgatggagggtattaatcgcagttggacagatgaaagaagggctgcgacttctgtgcgatctaaaaattttgcggctaaaggatgttacgataaatgtctagagaacgctagaataaaacttcagaatgatccagaagccagacgcttatggatagaacgccaaaagaatactagaaacaaacctgaattcaaagtccaagattcaataagagccaccgaacgtatacaacgtaaagaagttcgagaaaaagtctggctcccattctatctttcaaatagatgctactacaagactgaaggatggcgtctagaatataagaaggctctacaggtctgctcacttgaaatgcgtatgctgtatgataaaggatatggtgccaagcgattatgcacattcttatttgacgttgacgaagagtataaactattttcagcaatatgtaatgctatgcgcgaccctcatggagcaacaggtaaaccgttcaaccctataggtgatgttgaatacatgaattggaggtctggtcaagatgttgaagctgctagaaagattgtattcagagataagttggacgcagtcctagatatgttagaaatgaattagaatactaaagaaaaagcccctaaacaggggctttttctttcaatgagtcaaagatgctgtttaagccacctctaagccacctctaagccctgcaccaacagtttacggtagtagccgttgctttcagcgccaagacctttaccaacggtgtcggtaccatcagcgttcttggcataatatggatttcctgtaacggcgtagcgcgttttaaatgccatacgcggttgcatagaatcagggtcttgagcgcgatgcatttcaagcggagtatatggcacgtagtagataccagcatccaattcagttgcacctttgtaagctacagtcaagtagttcacgtcagcaaatgggtcgatgtagacacgttggccagaagctaacacgccgcagaaagtttgaccagtagggtctacttcaagttcgccgtttgcagcaattgcaggagagaagtcaagcatacccgccatagcaagagcagaagctacgtttgcagaagtcaacaggcggtttgcgcgaccacgacgtgtgtcaatcgcaactgcgttgttttccaattcaatacggaacaacaatgctttaaggcgttcaagtaaccaacgaccgtcagtatcagtagtaaggttgaatttaccagcttctgcagtaccagttttcgcaccaatttttgcacaaacgttcatgcgagtaatgaattcgcggttcatttcagcaagaatttcatttacaagaaggcttgaaatgatttcgtctacgttttcaccgtggacagcttgcatgtcttgacgcaattcattagtgtaatcagcgtatagaccacgagtaccaacagacaccgtttgtttttcgatgctgattgccatacgaccccaagcattgccggcatcagagcctaacaattcacctttggcagtagtcattgcagtaccagaagcaggagtagcagtagcgccagagccagtgatgaatgattctgtgaaacctgagcttgagccaacttcagcgcctgcaccagagtgagctgtatttggcttaccaaataatgcttctgggccagtttgcgaagtgtaacgcgaacgtagagcaaagatcagtgtgtctggagtagtagcaggctgagtaccgaagaaatccatagagataagcgcttgttgagcacgtttcgccatacgaactaatacaggtgcccaagttgcaactgcaccgccaccgatggtgttagtaggcgctgcttcattcaatacttgaacgtttgcattacaccatttttcttggttttcaagaatttgggccatgatgccttttttagacaaacggctaaatgcttggctgttcgcttcatctttaccttcgatgattggcgtccacttttctactgtttcgttaaattgatccatgataatagttcctaaagatcattgagttgatgaattatttagttgaggaaagttcgaccaaaagatcaaactttcttgccttgcatttgcaacaatgttgcttcagcgagtgaaataggcttctgttgttctgcggggtcagctgcagcaggagctttagttccttcgttgaactgatcttgagtaccagcattatcaacggcaccatcaacttgtttttcaacagtagaaggggcgcgatgagcttcagcaagcgcttttaccattacaccgaattcagcttcgtcagcaaaagtctgttttgaaagagcttcagtgattttgtcacaagctaaatctgaaagaccgtctgtagcttcagcaaagatttcagcgcgacgataacctgttagttcgtctaacaatgattctttttcagtgttagcttcatctaattgggtttgtaatgtttcaacagtaccattcaacataccaatcttgccgtctgcatattggtcaacagattcattgaaggctacggtatctacacctaaaagtttgcgaatggcttctgacgcttcattaaccacttcagcttgagcagctgattcgagaagagatttattggtctcgccccattgttcaatgatagtgtccaaagcagattctactaatccagcttgattcaaagcccattcttgtttagcggattcaagttgcgtttcaacacctgtagagatgccttcctgcagcttttcttcaaacgcttcaacaattttgtctgctaaggcttcttcaatacctgcttcagccaacagatcaacaattacttgtgacatttctgtttccttttcaaagagtttcagctcttgttacaactactaaggttgtaaaattttagccgtaaattttattgtgtaaagccatgttcaagcgctctaagaatttttcatgatccaagcctgcagcttgttcagctgtttcagcttcattaatcacttcaccttctggagtgcgttgttcccaaccacctgaactagatggataccacatagcgctttcgtttacaacatcaaagtagcaaggttgtccagatggaagatcaacggcgtcaaccgcattcatgcgatattgttcgaccgtgaacttatcacgagcaagtttacccataccacgcgtagaaacgcccaacttgaacccttcgtcaatcagcgcttttaatttgcgcccttcatccatagactcaagcacctttgccttaccgataacagtgtcgcctttccactctaaagagtcgatacggatagctgccatcttagggtttggaaaaggatattcagggtgtaaaaattccccgaaagcgcgtctttcgttgatgtattcagttttgtattggtctacgactgcttccatcattgacttagggtaaacgcgtccattacggtttttcatattacaactgacgaacggtccctcaatgtacatcgctttaccagtagaagtattctcattgagaatctctacttgatcagttgattcgcgtaaaaacaatagaccttgtgtcattttgagtacccatatgctttacggaacttcatggctttacgcatcttacgtactgtacgcttgataaagccaacgcccattgcctttttagtgcgcttacctgctcttgcacccaacttcaaatgagtcttcatagatgaagtcattggaacgcaagcagtaccttctgcatttggatgatagccaggaggacaagataatttcttgcgcttcttgcctttgaaattcaccttatagatgaatttctcaaccaaatcttctgtgttaatatcttgcatcgcaatattctcacttgttttcgttgataccgtcaccgccaacgaatttacctttgtaagccttttggcattcgcgcttcagaacagctagaccatgatctagacggtttgtaatagcgcctgtaagtgcttcagtgctattacctagagcaccaccttcagattcttgtacttgcaaaattgcttctgctagttcttctttaaaagccatttggatcttctccttcagtaggttttggcatttttgctacacgctctttttcacgcttgatttgctcaggtgtgaaacgaaggatgttttcatatacccattcatctgaaatatatttacctacatactcgtccacgcttccaagattggagaaccgattcatcaattggtctgtttcttgctggtcttcgacccaagaatcagataaccatttgaaattcagattaggagcaatatcgcgttcccattcttcgtctgtaacaatattttgagaaacagcttgaattttgagaagaggaatgaatacaccgtcagcgtagcgtttacgcttacgcataacgaacttattaaacttccattcatccctagtcatttcggcatttctgttgccaaataaagtaggttgttgatctcccaaacgcccttcaggtgtgtttagagaacggtacagaagtttttggaagtaaagaatatcatcaatttcagaaaggttttgagcgccagctaatgtagaaacgtcagaacctcgaccgccatcacgaactgggaaccaaaagtcttccaacatatgttgttggtgacccttaccatttacctttccagacttaggatcatactgcatcttagtcttgtgttcacggatgactttcttcatctgctcttcagcctgtttaggcggtaaagaaccaacatcaactttaaacacacgcttttctacagaacgcgtcaagcgatagatcacctgagcgtcacgtagcatttttaaattatttagtggttttttggctttttctaaatctgatacaacaacgcctgtaagaggatctttaacgcctgaatgcacgtaaataagggcatcctgatctagtatctgagtgtctaggcgcgtgatagcaggcattgacaaactgttactgaaggtattgtattcaggtgcccagaaggggttgaacacaaaacaatctttgatacggtcaattacttctacttgtttgcggtcaacgattgctgatttcttttcaatcatcttgaccttttgaacagttctagagtcaagaaataccagctttttgataccttctttgtgtctcgatggatctagaacaggctgaatatattgacgcccgtcaacgtaccattgtttgatacgttcgtagccgttttcattgaaatccattaaatgaagaaggcggtcaaattcctttctaaacttttcacacatcgattcgctcatttctagcttagagaaatcaagttcaacggcgcgtttgtcttcttcacaagtaacaatagaattaataatatcgtccaccgccatatcaacgtcagggtcaatagccatatcccgatattcattgataagggcagcggagttttggaacgtaccatctatggctaggttggcagattcatggtggaatccgctcaaaaccaaagaaccatctaatgggtctgtcgctcttaattgtaacggagcgagttttgcctcgatcgcttttgcttttttgctagtcaatccaaaccatgaagctgccatttgtcacctgccttagcgagtagtgttcgatgtaaactgaaggtaacgtagagtcacgttgaattcagatgagctgtccatagaaccgcgatccaaatcaataccgtcaataagagatggccatgcgtcttgaagggtataagtcttcacaacgcggtcatttacatcaagcatttcaacagtgatatcacgtaagaaatcagtgtagtccaatgaagtagtagtatttgattcactaccattgatgtattcttgccaaatttcaagcgtattacgcactttgaagtcattcacggctaaaagcgtgattgagaactcttcgaactgacggtcgccaggaagaggtacagtacgtccttcaaagggcacttcaattacacccatagttgacgaaggtgtttttgaagaacgccctaatacagctgcttgtttcacaacgtctgctgtacctgcgaactcaggaaagttaaacagaacacggaatttgtggttacgatacaaaccactcttcaccgaattcttcaagtcttgtactgacatgttttcatccttatgaaattgagggagtttcctccctcactctatttattgagcttcgttttcttcaaatgaagcgttagaaccttcagcttggaagtacaagtcaatgtaattgatgctgtactgaggccagatacggaagttagccacgaaacggttttcttggcgaacagacaatgggttgttattttcatcaacctgcgtttcaaccttttcaaatgcttcttgtgtaaccattttacggaagaatggattcacagcattcaagaattgagaacgcgtttcttcattgttgttttcaaagaagtaatatttcgcgtagttggcaatagactgctcagccacaatgaacgcagaacgaacgttgatacggctaaatgctgtaggcttagatgtagtcatcttgtcgccaagaagtaccataccgtcagagcctgcatcaataatagagtttacgccgattttgtacaactcatcgcgctcacccttttcagctgaccaagctaacttgttatagcctgcatacacgccacgagtataaccagcgtgagacttccaagcagacacttcgtcagtagtacgcgctttcagaccagctgtgccgccacaagttggaatccaaacaaagcggtcattgtattgatcatacacttgagcccagttgtcggtagtgaacacatacgtagagttaatgttgaaacgcacattacggaaatcaataacagaagccatttcattgccgctattgtttacaacatcttccatatctggagccacaaagccaatacagtctttacgcccttctaccacttcatggatcagctttttctctagtggacgcaaagcgccaaatagatagttgatcttgtagcgttcggcgtttttgaacaacgacaaaccttcgtagagggttgtatcgtagtcatccacgccaccatctagaacataatccttaggatcaattgttactagggcagggttaacgtaaatccatttcgactggcggttgataacatctagaatgtagttactagaaccgtcttcctttttgctaccttttgtcaaagacacagattcgtatttttctaacaatgtaccgtaaccacctgctttggttacgtttgaaacaatgtgaagaaattcgtcagtctgaatacttgggcgttcattacccagcacaagattacgcatgctgatcacggcagtgtttccactaacctttgtctcaacaatttggtatttcgtgctattggatgcggctgctaaaaacttagttttgatgtcagcaacaagatcggcaatagaaacacctgtagagtattcgattaccgcgccgccaatggtaatggtagacgttgcaactaattcaggcttgcgaatgcgctcaatgtataagttgaattcttgatacgcagaacctgttcctttgattttaccagttgtatctacaactgctaccgccacctgacctgcatctgggacgtattcaaaagaataagcgaatggccacgtagagaatgaagcgctgtctgccacgctgatcataataccgtttgctaggctaccaggatagcgtccagcaaaaggtgaaccagcaaagtccgcaaattcgagctcatcttcattacggattaaaaggtttgaatcaaccattatttacactccggagaaaattgttgagttacgcgccttctcgcctatcacacgtacaaagaacatcttgttggtgtaacgtaaaaagtccgcagcaatgaagaaacttagtgctgtctgcgcatctggtttgaaaaaggtttcagcgaactgagtaccaccagtagtgataggtgcgatttgaccacacttaccccaacgcgcgtaacctgccattgcccccatggtgtcaccctttgtttcgacaaaagtggtattgtctacttcatagatgttgatctgaggagaaatactgaaagccataagacttcctccttatttgatagttgtatatgttcaactgttattacttagtcttgtttggtcaaacgcatccaaacgattctctttcctttcggaacttcaacttggaacgtcgtgtcagcattctgattggtaacgtttacatcttgtacaactccattcacaattaattctactgtggcatttggttcagtcgtagtgtctacgatatgaagaacaggcgctgatgaaatagagattctgtgatgggcaacgttatcagaagcctttttatagcatgcgtttctggcttttttaccaacaactcgtacaaaagctagtttattgctatattgcagataatcttttgcgatgtaaaaggtctctgagcttggagatttaggaggaaacatcttctccagatactcgtcaatacctttgttaatgaatatgggcttcatgactgcgccccattgggcagtaccagacatagctcctaccgtgtcgcctgagaacggtacagccgaagaagtggcgtccgtctcaaaaatgttcacttgtggtgagatagaaaatgacatcgtatttctcctgatctatctgtacttatatgatttgaatagtgtattcttgttctaccttattgcctgctacatccgtagacttaaacaaatacatgtcccaatcaacaagttctatagaaaatgaatacgcgtttgaaacaagatcaatggttttgatgtaagacccatttctgaagatagatattgttttcacatctttggaatatatctccaaagtattattcacgcgcttaatatcaatttttggtagaagaagaataacctgaacatttctagacagagcatttacagtgctatggaaacgaccttctttagttctgaccattattgaattcattttatatgaaccgtgtttaagtctgaaactatttcctgagccatcattccaattgaaaccgccgtcaacggagtattcccaatcatagccttccgtatgcccgactataactttaccgtcgttggtgatgtaatctttgttagactctccgctgtcattgctcaatcctaacgtaggtataggaagggacttgttgcgatagtctaccgtcaaagaaattgtattctcgttgccggcattagatgttacgcccaataggtattcacctctggtaattcctgaactaaacaagaaacggtatacacctttactatcagattttgttgtttgaatgacgtctccgttataacgcatcaacttgacttcaacgcgtggaatagtttgacctctgatacgtttgtattcagaatcaattccagctgttatctctggtttagacgtttcaatgacgaactcagacataaaggttgtctcgcccaactctccgtacggcgtaacttgacgcacgcctactcgaccttcagagtagattccctcaggtaattggaatgaagttccaaagcccttaatgtaccgtgagccataatttgtcgtatattcccaagagtttcctccctgtacgcccttaacggatatcataccagtattggagatattcggcgcgtatggtttactaaggaatggatccaacttattgatgttgtattcattataatttggagctgtgtcagagaatgaaacaccaaaaccagctggatggaacatttttagataaacatcggtgtacaagggttcataatattctctagggtgttcagtctgtacgatgatactatattctgaaaacaacagatcatctttaatctggacattagaatctagaatgaaattatcatctatgcaagcaatcaacttgttagggttgatgattgtaacctgttcaccgtaaaacacattgaagaacaactccaaagcctttgatgttcccttcaattgatataatgaacgtaagatgcggctagtaagaattgcatctccgccgaaagcagaagcttcaggaaaaccagcatctttcatccaattttctatagttttcttatgaacaaaattggattgatatgtttccccgtcagacgtcttgaatatgttttgttcagatgtcaagaatgactctggtatgcgttccatatttaatattgacatcatattaacagaccaccagttcggtgctctccaatcagaaacgtgtatatcagctgtttgttgatctaaaccactaagacgttcatccaaagttgcaacaaatgcttctgcatcgtctcttaccagctctgtctcgccattttgtatgcgactgcgaataaccgaattcatgaacccagaatcgttcatgaattcataatacgtttcaatgaatgttaccagcttggggtacatttctttaaaatgaagcggtagatgtttcccaactctcagatgattcatagtgatactccattaattgtaccagtacccaattggaagatcacacctttagtcgctttcattgatttatctagagcagaacgttgcgcccaagccttcatcttttctagttcatcaatgtaaaggttttggatgtatactacacccttagcctcatccacacttccgtattttccaggatatactgaaacagaaccagtgactttattcactctttccattacaagttctttccctgagacaaccagtctataatcattttggtcgctatttgtttcaataggcttaaagcctgatactaacacggacgaacctagaatagctgagccatattctattttcccgttgtaataatatccaacctcagcttccgatatttctttgtatatcttgacctgagtgtcagcagatagaatagagttatctatagacttgataaaggcattaagcatattatggtcaaaatctgtagagaaattttccagattttttgaagagtacgtcttcactgcttcttcaatttttctatctaattgagaacgagaaagaactgtgcgctttggattgtattcgtagtctacatcaactgacagatggaaaatatctgcgtctttgattaaaggcgtaatctgtccaacgcacaaaggttgaatttgttcttgtagattatcctttgtcatttgatctaggtttgtgccgtttttatatcttgctgctatgaacgtgtaaccatactttggtgggtcattctgttctccaccccatgcattcacggataccgagtcgcccatgaagcgctttactgcagctatgaaatcggaaggcgtaacgcaaacgccatttgagccgaaccctagaggggctaaacgttttatggattcaatactttcttcatcagctccacctttactgtattcgcttaacacagttatggtaatatcagtagacccaccaataagaccactagctgataatttagcagcgccattcccttcagaacccagcgtagcaatataatctacaataacggattcaccatattgtaaacgtctactgaatacgtcatcgccgaactctagaacgtattttccttcagggttgattctgatgtaatacagtttgttgttctttcctaactgattgggatgttgatatctcgtataggtttggagattatctccttccttcaccataacctttaatgtattgatgtcaatacgctggttaggaatgatatatgtttctataccttcagatgtctttactacaaatcggtttgtgatacgttcaccttgaagtagtttaacctctgggaatgtgtaatacccatcaactagattagcgcggtatgtaccatcgggtacgaaattcaatgatacctgatttgaaacgccgttgaagcgcacatcacggtcaaatgcaatatcttcatctggattgattccagtagctctgacatccaacttgactagaatttctgaacagcgctttgaagcaggaaggtagttcaatataccactagccttagctgctacgttacttctgatagtggctgaattgattgatgattcattgaacgcaaaaccaacacttgaagcattgatagatgtattataggcgtgaagatcgaccatgcgagcggcaacagaacctttccagttcacatctttgaagtcaggatgcgtagaaagaaattgctgtaatgcctcttgcaagtctctgaagtcatagctgtcaggttgtctgttgctcatggctattcccaatgtgagagttcttgtactgagaatatttacacttgaaataaaaaggagaccgtaatggtctccatatttgataacacatttgattataaaggttggaagtattttttcaccaactcataaccagcatctgtaaatgtaacgtgtgtattgccgcgtcttggatttttgattacgattattaaacctttcgattcaagattatgggtcaaaccgcgaattgctacagggtggaagcctgtgaagtcagatatttctttaacattcaatgatctgatattaaggtctactgggttggtgatatcacgaatatgaataccacgctctacgccatcagagtgaacagccaatgttttgaaaagatgtttttggttgtctgttaatgtagtcatttgatttactccgttgagtgttattgaaccttatgaatagtattatagtcaatattcaattaaaagtaaagtggtttaataaaaataattgataaatttatttcttgtattccttaaacatagtattggttcaaccccaagacaaaacatataataaagccccaattaaggggcttcattttactattttgaccagtattctctacaagcttcgtttacaatagtatagaaatcatcttcgcaattttcaactatttcagaaacactatcaacttttctaacagttacattaccgttaacaaccttagttatagtgaatgattctaggttattgcgtacaactgcaactgttaaagcgccgtatttggaaatggaagtgaaagaggcagagtcagcatttaatgtaacatacacatcttcggagtagtcataagaattgactagactagacatcgctgtcacaagttcttcagagaattttaaaccgttgaaagaacgtccaactggagaagatgagctttgctcgaaagtgtattttgaatcagagaagttgaattgaaccttagccattttgatttactccgttgagtgatattctgtatatgaatagaattataatcaatattcaattaaaagtaaaggattatttcccgaattccagactaccgtttggaacccactattttggtattaaaaagccctgtactagacagggcttaaaatctaaattttaagatttactgttacatcattttgacttcgcatcttcaaaaatatcaaaaatacgaggtaaagaatcagctgttgttttatctagacgcaattcgacaaaacgcggcaagaaaagactatgatcatccgaaccatcttctttaggtttcgtgatgtcattaaaccgtacagtaaccaattggccaatcaaattgtttccaatgtggtgtaattctgtacgtacagcgtcttttaagcccgaagaacaacccacttttaataaaccatcttctgattctaattgtagagaaccaaataaatgttcatattttgaacccttttcggctggattaaaccctacaattttcaattcacattccgcttcaatcttcatcttaatctgaagtttattgtcgccatcttcccatattgcattagggtgtttgatgatagtgccttcaagaccagcattgattttttccaagaaatgatcaacgcattcttcatagctgttgaagaatttagtttcaataggaaataccaatgtcccttcagagaacaagcgcacaatggcttcgaagcgctgtttgtatcccacaccgaatgaacctttgtatttacgcttttcagtaggaacaatgtcccaaatgaagtatacaagttcagcgttttctggaagttctttaccagattgttgaaggctgttcaacataccattagattcttcgcgcggtaaaatctctccatccacccgcactaaaaattcaccttcaattgtacagtctttaactttgtattcgttgaaaacaatttcatcttgttttaagcgcacttcattgataagattcatgaatggaacgctattagggtatattgttccagctctggacgcgcaatggtggacttcatcagaagcatttaatgatgcaaatgatccgtccgctttcaactgagatacgtagaagccccaacctttgatgtcactttttgtatctttggctaatacacaacgctggtacggttcaatgaaaatactatttggccagattttgatcaaagttcctgatgccacacctgctttgatatctctgcgaatgagagctgcaaagagttcttgaccttctacagaaagcgtaccagtaaattctttgattgcttcttgagccgcatcacccgtcaataaacgttctgacagcgcagatacaaaatctaacatacgttcaatttcagtatcatcgtattcttgctttgatggttcagcagcttcatatttctgtacgtaaaaacaaaggcgtgtatcatatacagtagacaaatacttctgtagaattttgtttccggagttggcatttaatagaccaatcttattgtttgatcctttagcgttttttacagcttgtacaatttcaaaaatgttcatcaatgattctccaaacgagtgaaggtattataacctattttaataattcatcctaaaatcaaaagggaagccgaagcttccctttatcattctacagtttatgcaatatcaaagcgcaaaccaccgcgttcatcttgtacaaggtcttgacccaacttaacgcctagcattttgctgagataagcaacattacctgaagttggtaagatatcaacaacgccttcattcaatgatacagtgaaggtcgaaccgccgtacgcaaatgaagattggtacatacccaattgtcgcatagatttttcaagtaaagacttgtcaaccttgttttgcttaggagcaattccagttaaatcttctttgattgattcattcatcattccaacctttacagacttcaaagacttgatggcgttgatatcgtatttcaagaagatggctttaacaagattagttttcttgattgtgtacatccaattagagataccaacatatgccatatagtcattaacaacggcatcagggctaccgttactgaacgagtctgcaactgtcttagcgatacctgcttgcgtcacgctcacgtaacgaccatttccaatgtgagtttgcattcctgacggaatttcaacaccttgttcagtgaacttagctttgaatttcttcttagagtatggctcagtgaattcaacctcgaagaaaccaacgaaacctgtatacactacagatgaacgcatctctttgtaaatctcgtcaatttgagcttcgttgcccgcaacatagtaaacaactccaactgtacctgtttgaccgttgtacgtcttagtaatacccacagacaagtctaacccagtcaatttcttagagtcgcctactgttttcaacttctccaaagcggttgcgatagggcgtttgatgtccttgatatttgaatttgcgcgttcacctctaacatcaatcactcgaccacgttgtccaatcatctcttcaaacttagccttgatcgcattattcgaagctccggagttgtacatgcgcagaatttcatctttcacatacttctctccatcgctatatgtcatacctttaggggcataacgatagaagccgtcagtgcgtttggtgtaacctgtttgaagagataactcgccatctttcacatttatataatcagtatccattgacgagtaaagcgaaacaactacttccaattcacctgattgtttaggtagttcgccagtttgacgtctcttagtatcagccaattcataccaacgcaactgtaattccttagctttggttccaatcgttttgtagtgactcttggcttcatcagccaaatcttgaaccttagatttttccgcctgcaatctcaaagagttgtataaatccgataattcgtttatgatgctattatattcagattccatatcaataatatctgattcatatttgcctctgaactcttcgtctgataagccggagagttcatccggagaatatttcaccatccattttctaaaaattttgtctcgaatctgggctagtttttcatccaaattgtttgacaatgtccgtactttaggctgagcagctgccgatccaggagtagcaacatgttcaccttcaacatatccagcggcagttagagcattaacaacttgctttttagtaccaactgttttactgaagtgtgacaacttcacatacacagatccatctttattatcagtaaggtctaattgtagatcattcaataacaacatagttgctaaaggagaagttgttacaatcttgaaaccacccctttgatgttcaaaatgaaacttctgttcaactgtattgtctacgccagatatttcagatgcagtagcttcagataactctttatccaagaaattgtctaaaacggcaagagtgtcattgaatttcttgtgacccatgtaataggtttttagaagagcagactcatccccttcattccaaaaaggtgtatcgatcttaggaagtttcattcctttcacgttgataataatcatatcgtcactaatggtaactttcaactctttaccatttttatcgattttcaactcaccgccgttagctctcgcgtcactaatgtattttgaacatctgaacgcttttaggtagttaccctcaaagtcatagttgtccagaacctcacaaagactatagaactcttctattgtattgatatcatcgaaacctacaactgacgacactttactatattgttggtatttttctaaaccaacaagataacaagctacatcttgcttcaagctcaaagaagcgtaattcttagatggttggaaagtcactcgtcctggtttaatgactttaacaggattaaagcgtttagataacacaccgtagttatcatcgtgagccttcagaaacttccctactaaacctgcatcgaactctttaattgagaaattaccatctaatggtactttcaatgtgtttaaatttccattgacctcaaaatcaacagaaccaaattgttcgcgcgtagatttagcaaatggcccagtgttacttgtaaacactgcaaaagcagttccgttgtcgtttacaacaacttcacctttgaattctcgatagtctgaaatttgaccctctggtgctccgatacttctttcgtacattgtagttgaaagatcatattctctgtgagtctttgaaatgatagtatcaaactgttccgccacctttttagagatagcttccttaatagacaatttaccaagtacgccgatgaagttctgtgtgagattatctccattagttttatccacgaattgacgtttattatcaattgtatcaaactcagatttgaaccacccgaacacataatccatgattgaattaaagtttacatcagccttaaatgttaattttcgaccattcacttcaaagacaaaaccatctttggttttgtacgtcatggtttcgttatgttgctttattgcagcacctagagccttgatatgatcaggcacttcaggcggatggaagacagcttctgcatgttcatcatttaaagcagttgcaatttcttcaattttgtcattccaagttagataaccgcctttcacgaccatagagaacatgtcgtcaacttgcgatccatctgcgcgtctagcttgaggaataacctttttaacaattgcggcgatcgcatcctgaataccagaacgttccatttcagaaggatgttggaatacaatatgagcagtacccgattcattaccttcatccactgtaatatcaatgcgcgcatcttccaatgggttagacggatcattgaggtcattagcgccgagattggtcttttctatactaggattaccaagtaactcaataatacgagcaaccttgacatccatctggcgcccagttgcattagctttcaaagatgagattgtattactgtatagacgcattgtgtcattaccgtattgtaacgccactgcacctttaagaatatcgtatgcacgtttagcatcagcgtctgtgctgaaacgtaaaaggatatcatagcgttcatcagttaacaattcgtatttgacatcgtcttgaaaagtaaattggtctaatttattaccaactaattctgattctatgcccttattttttaggtttaatatcaaagattttatgtaaggtttcaaatcttcatattttgttccgtacacaaaagaaatgatttgcgcgccgccattgttgcgtataccatctttttctaatccttcaacgtcttcaaagacttctcgacaagcctttgaaaccagacccataattttaacatccctttcgatagaaatgtacggcactggcccttgtaccataatcttaacggagttaacatcaggctcagcggaagctttcttgaattctaatgtctgttcatattgagatgaatccgatccattagttttatcttcagctgagttgatccaatttacgacagaagtgcggtcaaccttgactttatcaccagcatatgtagcagttacgtatgcatactgacgttctaacaccttcccattctttttaacagtaagacggaatttatacagagttacgccagatttaggatcaacaaattcaacaaaagctacgtctgtgacgtcgtagtcgctttgtgacaactcatttttaatagctttatccaaaaactgtcgagcttcggcaacatatttttgggaatgggaatgtaaatcttcatccttgtctctaacttcgacaggcttttcgggaacaggcgtattttctgctgactgtccaccttcactgcgtttatcaagaatatctttcataatcttgccgttgaagacagaatcgaattgataaggcttaaccgtagctacaacgcctttcaaccccatcttttcaaattcttcaatagacaattcaaggtcttgaaccttcaattctgtacgcaattcgcgtttgatgattttgtctaccagacgaccgtgagcgccgaacgcttgctcaggaacttcaataatgagaccctttacatctgaacgcttttgacggttatcttttacaatatcaatcaatgtagcaatgacgcgcagcggattatggaacttgccaaatacgcgcttgtacattttgatgtttggagacttttgccctagagtcaaaacaaatacgttacgaccgtagttcacgcgcttctcaaaatggaaacggaaactatgatttgtttcgtcttcaaaggtataactaatatccatcataccgttcttttcagtacgttctgtaggataagggatggaattgaatgcttccagcaaaatttgtgacatggagtagtgcctctcagtaagttccaatgtaaaaactttacatctatttacattggaacaattattactgaaagtttaagagctatccaaaattcaattcagatatgttcattgtcttggtttgagtctttttagctgctgtggcatctacgtgagccatggcttcttgaatctgctcatccatgcgatcagaaacgtctttatattcttcgccttcaggcaagtcaaagaatatctgcaaatcgtcattgataccgagcgcgaactttggaatgtgcatcttgttagcaaaacggtttttcagaacaataccaattgctttaccctgttgtttcagctcattaggtgatactagagccaatgtaaagtcagcagtagcctgaacgccaatagataaccctgagttagacaacttaggatccttttcatcttgtccagaacggttgaactgttgagctgaccaaccaatagcatccatctcaatcattaaaccgcgtagttcctcggcaacgttggtaaagtacgtgttcgagtcactctttgtactagggtggagtacagatgagttcaatagcgtaaggtaatccaccatgatgaagtcgggcttcttaccatgtttgacctcaaattcttgacacacatgacgtaggtgacctacgtgagctgaagatgccgcaaactgttttactacaactcgaccaaaattttcatctttgatctttttaacgcggttcaaataggcaaaagaatcgtgcgctctgatctgttcagaattctctccgaacaaacgtacatccaaacgctcgcgtaggacttcttcggcaatttctagtgtaaaatagataccgtctagaccttgcttagcatagtcggcagccatattcagcatccatgtagttttacctacgttgatacctgcctgaacaatggatagcgttttacgtgtattaccaccttgagtgactttattcatacactcaagtttgtaaggaatcttactagctggattctgataataatcccactgtttctgagcgtcatcaatatagtcatggccaatagattggtcaaaacgtattgcgagcgccttctgaatcatagaaggaatacctgtctttgaaatgctactgtcttcgccgttcatcgcagctagagatgtagacatagcaatatacaaagcgcgttcttgagcccacttttcagtttcatcaaatgtccaatctagattactgatttcatcttctggcatataaagcatctgaatagctacgctagtaggatcatagatggcttcaggtagattggtattgcttaggttaataagcaaagcgtccttagtcggtggagctttgtattttttgtaaaaatgattgattagtttgaaagtattacgttggccaatatccgtaaaataatcaggttcaataaaattgattactttgttcgcgaactgaggcgcgtaaatcaaagtactcaacaataaattttcaaattgcattccattttccccgaaaagataggggcttgcgcccccatcgttatatcacttacgcttcaactaaatcttcagataccatctcaccattttcaaggcggtatttgtcttcacagaattcatgaaatttaggtgacgctaaaagtgcttcaaggaaaccagattcacccttgttcacaatttgcttgcgttggaactttttgctgagattaggcgtttttaattcatcagccaattcataccacgcgccagcctgtttgattagtttagattccacacctagttcaatcaagcccgaagcccaatcaataccgccatcgaatgtcacgtgcagtttgaacactgacttttctttcacaaaacggtgttttagggctttatagttgaagaaccaacctttcaaaccatcatcatctttatcttgggcacgagaaacaaaccaaataatatcagatgaaaggaagttctgtttaccacctttaataattgggtctgcatactgattagcattgtcttcatagaagctgttaatagcaacaagaggaagatcgcgaattgctaacataggtgtaatgatacggaacaaagagttcatttctttggcgcgcgtcatatctaccttcacgtcacctttcaatgcgtcagcaacttctttaatagaagcaacctgagagattgaatcaattaggaagaataccttttcaccgcgtttgacgtcttggataatctgagtgagcgtttctttcaattcttccagattttcaaatggaatatatacaacttgctcaggtggaataccaaactgttccaatgtcggacggatagcaaattcagagtcgaatacaactaaaacaggctgcttttctaagtcatttgggtcataatctttattatgatccatataacctttggccatcattaccgccatattggttttaaaggtacgtgagtcgcctacaagctgatgaataccaaagcaaagaccgccgttgaatgaaccactaaacatcaggttcatgagaggaataatgtatttagccaacatggtcttttcaccaatatctgtttggttcacaatgcatgcttttgattttggagctgctttgataaggcgtgatgctaatgacgacatgtttattcctctaccgtagtgatttcattttcagctgctttatttactaataagccctgagagattttttctgcgcgttctgaattacgttccagaacctgcaacatagtattataaaactgttctgctgtaatattaaaataatcaagtgtgatattaatttcttccattagacgttggaagtccattgctacatacggcgtatactgaagataagacatacccttggttttgatggtctcgtaccaaacggcgaaccccacgtttttaatactttcgcgcggttcaaagtaagcatcatcgtttacaccttgaaaatctggatatttcaacaatagcacagacatcttgaagcagatatagtcacaaaattccacaacggctttgtttacatcaaattgttgttcacctttcttccaccaagcccactctgtagtgatctcatctaaaaattcagagaactcgcgctcagcggcagcgcagtatttgtgtagatcagccttttggcgccagtctgttgttcctagatagagtgcgttgttgcgattctgaatttctaacatagtattcagcatctttttgtttagacgcgtaggcatcccaacattaagattcaaggcgctaataaaatcagccacagatttttcatcacgaagattcttaaacagaacgtcgtaaccactaaattcacgcgtccacttttcaaaatcatccggaggtaaacctgtttcggattcaatttttacaaacaaaatgctatcaaatgtatgagccgttttgcgtaatagatcgtattcatcttgagtgcgcatatccgtaatgaagaataaatcatttgacgggctttccagaattagatgagactgattctcagaccaacgtttggcgaaataagagctatcgacatctcgacagacctttcctatcatcttaatgatatctcgccaagaaaactctttaggattgtctaagttgccgataattgggtcattcttattgtcattcaaaacattctttagaaatgggaacaacacgtatgccacgcgacgcacttcatcgctaaaagaaagacgtgtaacgttaataccagatttagccatttgttcagctacagctgatgtgaggaaatcttttcccacaccgcgttcaccgcatagaacgatgagttgacccattattttgtctccacaagttggaaagatacgctttccgtatcatagttaatatcaaagtttttgaaggctgagccttcaatagcagctgctaaagaatctggaatactagagatattcaagactaatttctgatcaggaagcaattctttaatctttttacccaaggtgtatgctttgattagaacataatacaattcaataatttcattgaacttgccaaatgggtaatggttgacaagagcgcgtagatgctctacgttatcacaagcgatgatttcgttcagggaactcaacttatgagcaaacatttctttagtaaatggataagctacaggcaaagtaccttcaacgtgctctttatcgtcatcgaagtgtttaattacatcttcagcttgaagatagtaatttgaatagatatgagcatttgtaacgtgccagcggtatacacccagttctaggtcttcaaaaccttgaagctttaacagacgctgatagatgatttcttgtaagaaggtgaattcaaacggattgatggaaccagtaccgaaaatcatatcgccagaacgctgaataactttacaattcaacttattgtctgtaacataaaaatgaatttcgcggttacatggaatgtcttttggtttatgaccttcaccatacaagcgttgaatagtcgttagattatcttgcattgggttagcgatcatcaaaatagcacggcgtgtatttttaccttcagaacggaaggtctcaatcaagtcatccatctgaccatgatcatatatacgtgggccataagctccatgccaatgagcgccgtcatcagaataatctgtagcacgcggcagataaaattctagaaatcctgtaacttgatcttgtccagccataacccaaaaagattcagccatcaattggaagatgttacttttgcgtccagttagagaaaggtggcgctcgcgtgggttttgaatttccaaagtgatgtcatttagaaatttggctgtaccgtttcgagtttcaatttctttacccatacgcataatagaggtgatagcgcgtccaacagtctgattaattgttagtcctgtaataaacataggcggttgcatctttttctcctagagcaaacatttgacacctaatattataatgtattaggtgtcctatctgtaataatcaaatagatttattatactgtataaaacttcgagtgggtattgtattttgatcttggacgaccttctccattcttcactctcagatatttgtccatctcgcaatgggctgactcgatacatctagcatctatctgagacttggagattaggtgataaggaatcaaaccttcatcatatgccttatgcacagcattgataactttcaagctgtattcattccatacttcttgaccccaatttttctcaagtttggtttctccagtcaatcggttcataccacgatttgcgcctatgcctatattgctccaagtgtaacgatcaggcgcattctctagataccgcgtgtgattcaaatcagtgaccaattcgtaagccataaacccaccccaatttgggtattcttggaatagattacaagctgcttctagagtgtattttggtttactgagttctttctcaatacggtctcggtcagccattagttgacctaccactttacaacatacaaattcatgttttgttttgttgcgatacggagatgtcttatccccttccgtattgatcatatatgaagcggtatacaccttttcaccgcgtttatgacgttctagcatggtatcagctagaccttgaagactaaattcaggctttccgaaccagtatttacttttcaccagttcttcaatcataggtggccagttgatccatctagataaagctaacatcaaccaaaggtctggatgatccgcgaatggttttcgaatattgtctctcaaccatgtggtgactctatcatcttcacggtctacgttacaaaaacgatattcagaaaaaattgcatcttcagtccattcaaactttggtagacctgaatttttctttagcattatgctctgacgttcagcaataaaatatagaatccatcctaagtcgaaatcatattcatgtttgatgtctataagatgtttgttattcatctgcgatgttccaaaacccgatttttaaaacttagtgcctgtattcttcaaaaaggtagacggcagaacatatactgtaacctctacacgactatcattaaaaacagcgttgataattgaacgtataatattccaatctcctccacctagatcagaaccaattttaggcatagcgatgcggtcaatacctagacagtaacatgtctctttaatctcttccatacacattaatattgcgctgtaagagatggctacatctccttgttttttagctgttttctcttgggtaaaggcgttaacaatgtaatttcctccaccgacatcaacaaactgagctttacccaacggagactcttgtttaatgcgacactcgttgataaacttaatgtattcactatgcgcttttgggaagtttctacgtatctgtgcagctatacctgcacccattacgcccatacaattcacgccgtgtacaataactctagaccgcgtagaaaatacattaccaacctgtatcttatcatcgcccagcattttcatgaccttactttctccgccttcattaacccagcgaaaaagggaaatatttttgtctttcaatacaaggtctgaagccatattgttatctatagcgtaacatatttggcgcatagcttcaattgaagagtccacttgactcattgttcgtttttccatagcattcaatttagctgagatatgcatgatgtttatccttaggatggtagtataaattagatggtatatagttcaatccaaacacgttgagcttagacaatggaacattttattgagatattaaaatcatcatacggttattctaactacttaaaatggtattcatctttaatcaataaaaagattagtaaatcaactagcgatgaatacacagagatgcatcacatattaccaaaatctttattccctgaatacaaagatgataaaaataacctagttcaattaagtttaagggaacattttgtcgctcatcttttactttggaaaataaccaaagatagtaaaatggctctagctgttttcttgatgtctcaaaataatatcacgacaataaataattccagaatgtttgaaacaataagaagcaatatgtttacatctaaaggaatggtatggattcataaaggcgatgtaagaaaatacgtgcccgcagatgatgtcaataagcaactggagttgggttggtctttaggtatgtacgacgttaaatgtacttgggtctacaaagacaacttaaagaagatgataagaattaatcaattgtatgaacatctagataacgactggaagttaggattttcgcacaattacatttgggtcaacaaaaacggcaaagctgaattcgtagactcttctgaattggattcttggttaaatgacggttggattaaaggtattggtttcggaacaacctcaaacaaaataatgatgaataaggatggcgtgcgctgtttcatagataattcagataaaaattcatacatttcaatgggttggggcgttggtactgatctaactcatcgccatttggggaaaagaaaaatacaaaagggtgaactgataaaattcgtcgaagaatcagacctagaaaatatgttatctaatggttggaatatggctgaatttggtttagacggtaaaagatggataaataatggcacacgtgagcaaccaattctagatacagacgatataccccaaggatggcgtttgggacgactaagctctaagaaacaaggtagcgtctatatgaatgatggtagtcaaaataaaatgataaatcaatctgatgtggatacgtatctatctaaaggttggaagtttggcatgttgagatttaaaacagatagactcattccgataaataaaaacggagttaagactagagtgcctgagaatgaagttgaaaaatacatcaatgaaggttggaataggggtactggatgtaagggtaataagaacgttaacatgatattagatggcatcgtaaagtcaatacctgtcgagaaaagagaagaacacgagaaattgggttggaagacactttcttctatgtctaattggattaagaaggacggaaaaagaaaattcattagaaaggatgaaacccaaccttatttaaatgacggttgggtttacggtagataaagggcacgcgacctttacttcttcaataacataacttcacaaactgacttccaatcatccgaagtcttaatataactgatcatctttagaatagtacgtagagacatctcgcgtagtttatcagaatgttccgataaccattgaatcatctcttcaccttgaccctcatctatacctttacccgatagaatagtagaatctctcacaatctgtttaatacgtatgagagcttcgcggttgctgtgaaccatcaagtcaaggtagtttgcgcgtcctacaaatgctctgagatggggcgcggcagatgaacctttattaatttgtttatccatatcaatattggtgatgaagattaaagtaccattatattcaaatgtctgaggaatatcattgtcttccatccatctgctggtagaataccatgatacttctcgtttctcgcctgtatccaaggcagcctttaaaatagctagacacaattcgtctttgaatgggtctacgtcatctagaagtactacttgcccctcatctttattctcataaagcatagtatagagctgaagtggcgtaacgtgacctttaacgtgtttaatgctttcaatctgtccatcctgttcagcctgaataagtttagtctccaatgtatatgttttaccaataccagatattccggatacaatgagagaacgtgtatgtccattaatcaaagaatcagacaataaagacataatctcgaagcgcttgtagatcaatagatcaagctcttcatcagttaattctacgataggattagcaactggttgactagcagagttatctacagcgcctttattctgaactcgaccaaaaggaagttcgattgtctttgcgttttcatctttgaaaataacatcttttgcaaagatatgcacaccaaactctttttgagtatctgggtggatgatacgaaggtagaagtcgcgtttacactctgagtctttaggctctttagcagccacaacatattgaccagaaatttcacgattgaaataagaagcagaagagataaaagcgtggatagtcattttggttaactccgttgagttgatattgaacttatgatagaaattataatacacttcgccgcataagtaaagtgtattatataactttttcgaagttttttataaaatcaactcaagtaaagtaccttgtttagatgactagcagtcataacatcttttttgtgttcttggaattctggaaccattttcacaaagtctatgcctttaccgcgcttgcgaatttggaaaactggtttacctgatttcgcgcactgtacaaatttgtattgcccttcagatacttcgatcaatttgatgttgaagtattccagtttcggcttcaattcttgaagcgctttatattctgtgaggtatttactgcgtttgtactctttgtaacaagatgcaagaatcaagatactaactagaattaaaaaagaagaaacagccaaaacaacaatattcaaaatttccatctcggaaccctcaaggtaataaaagttgagggactttacgcccctccgtatgggtatattagtaagttaaaattgattcaactttcttgaagtctagtgtacgaatagcgtagcgcacttcgccgtctgcacatgtaatcttagcaatgttataacctttccacattacgtcatacattgcttgaataactttgtaatcatcaatctgatcttcatcaagaccaacagcgtataatgcttcttgtaattctttgagagtcatacggcgctttttgagagctacaataatttcccaaacttttgagcttttgcgaactttcagaagctcttgaggtcctttagggttacgacgcatccagttcacgccttcaccagctgtaaggtcatctgcatcttcagtttcttctttttccacaattacttcagctacagcagctacggcttcgacaactggttgtactgcttcttcagctacaatttgagagatattttcaaaagaaggttggattgcttctgtgataccgaatactttgatgtcgcggtctgaccaagggtacatttcgccatcaacttcaaggttgaaaccagcagaagattttttgtttttgatgatgcgatttaatgtcgcagcagtaatatctagtgagttatctacacgtagagttttagcaacttctttagaagtttcaagagtaaggttgtcgattgtaacagttacagttgaatctgcgatgtgtttaagaatagctttcatttggtatactccgttgagcgttaatttatcaattgaggcttggttttgattttgtaccttgcctttcatgattactattattgaattattacttgaagaagtaaaggaaaaattttaactttttccgaaatatttccttcttcaagtcttatccctataatctacaggtctaacaggttgaatgatgaagacgaatcttcaactttcaggtacggatcattaaattcaatgtcatgaggtagaggtctcggcatgtgtttcaaaagccaaccagcggttttacccactttatagacttcttcatacttgcttctcaagtctttacctattttatcaacgtgagttcctttgtacatatgcacaatgccacatagacacgtctcaatctcttgaatatcaaccagacggtctagagaaggcggtgccataagaccaatgcgttgacattcttgaatcatatgctgcagaacgcgtttgagatcaaactcatcttctggccaaagttccgaagacactaatttagcgcctttgatcggagctcctggtagattctgtacggcgtagtctagcgtgattttacattccataccatatacgcggtcatataaatctaaccacttccaagtgaagtatggcccaaaagacggaactttggcaaacttctcagccatcttttcataacatggatcatacatctcttcaataaactgagtagatgtttcaaaattttcaacaagatagttgagacagttcattcccttcttttcacggaagaaccgtcttgcgcgcccacgcttcaatgtctcgtagttatcaatgagataattccacaattgattagtaggtagattcatctcaacaatcttacagctttcttctaaagaatacaaaagagcgaagctaaccatgaagttgtcgaattggtctcttgttatctcttgagacaagaacgcaccgcgcataccactatagacagggtctaaatcaagcgtagtgaccatggcgtggcaatagtttaaccatcgtggtaacaacggttgatataaatcagccattaggcgtttccttgcgcattttgtaaatagaacggaatgtccagttgttagaatcacggtatttcaccacgcgcaaaccgtgcataaaacgttcttcacttggtttttcaaggacttctttattaacaatggtgcacaatccccattgctctaacatataagctacacattgagtgcgtttgatgtcagcttcgtctagtttagactgatgtccatcaaacataaacatcatcaagaagtgcatcaagtaatatttgccttcatggaagaaaatatgacacgattgatttagcgtgttagaatgagaagcgctttgaataccaacgcgtttcaaggtttcacgaatgcggttgaagttattgttgccgtcaacttcagggttaatctctacacagtacggaagaatatctgcacccaatggtcgttctacaagaatgtctactaattttttcatatcatttaccacgtccagctttaccgcctttatcaaacttttttaataataccttacattcgtcaatcgtaaggattttcaagtattgcaaagcaactttttcgtttaccttatagaattgcttcacgcattcaaggtattcacttttgtcatctcgctttttccatttggcacgagagttgttctttttgatgactgacatcatataatcatgatgcatttctttatcaatcaataccgcctgattagctgactgagcaatcatgattgtacgctgattctgcatcaaagaacgattgatcatgaaagggttgtattccttttcataatttgactgaaagaataggctgcgttcctttccagaattgatattgttcaaaatatcaaacaatccaaattttttctctttactagctgcggcttcagaggtcatcatctatatccttcacattaccttgacaaacaggacaacgcccatctacaagttggtaaaacacccatacacacgggcacttgagacaggcgtaccagcaacccattatttaaactgcgattccatcatgatttgagtagccatcgcgcttaaatgtaagaagcggtctggaacggtagcatgatgacgttggtaatcattcagagtaataatgatctgagcacgagattgatctgttagatcatctttcagagaatcgtagatgaaacggtagaagtcgtcaccgattgcatctgaattgtctagacaccatttaagagcagggtcgaaatttttagcgcgtagaatatctacgaacgctttggtatcgcgtgaattatgtagagctagtacaccagcgtccactttaccatattgacgcacgtattcttgaacagtatgaatcaatttacggttatctggcacacatgcttttgttaaggcagctagagtttttacgtcatgctctacgccttctaatttaagaatttcaatcacgcgttttagcatcagggttgccaattcaacctgctgttctttggtgtagatgaaatcaatttctgtacagcgtgagcgcaatgggtcaatgagacgcgctttgttgttacaagttaaaatgaatgaacagtttttagatacttcttcaatcagggctttcaaaccatcttgagcagctggtgataagcggtctgcttcgtctagcacaactactttgataggcgcgtcagagaataaagaggcagtggtagcaaactgagtaaccaagttacgaatggtgtcaatagagttgtctgtagaaccattaatgaacaacttttcacatttcaggtcttgacacagagctaaagctgtagttgtcttaccagtaccaggacttggagaaaagaagatgaaagaaggcatctttgcatcttgaacgcttttttgtagtgcagctttaaatgaagctggtaaaatggtttgttccaatgtgtcagggcgatatttttcgcaccaaagaaaatgctctggttttgatactgttacatttgacattgtttgtctcctaaaagttaacattattataatggttattaataaaaccagtataaatcaatttattacgctatgaaaaacgttatctacaaaattactaacattaaaaccgaccaacactacgttggtaaaactaccaaaacaatggaacaaagacttcaccgtcatataagagaatctatcaaagaaaattctcaaacccatcttcacagagctatgagaaagtatggtgtagaacacttctcaatagaacttcttgaagcggtttcagatgtaacaaaacttgatgaaagagaaatacactggatttcttccatcaaccctcactacaacatgacttccggaggggaaggcggtgacacatcaaactctgaaaattacaaaattggtatgtcaaaacgtcgatccatggctggaaaagataatccaatgtatggtagaacgcgccctgataatactagaattcaagcccttgccgccaaagctgcttcgaaggccaataagagagccgttatgtgcgaaggcaaaagatacgaatccattcgtgaagcttgtttagcgtatccaaaaacagatatacgctcaaggttggacagcgacaaatacccagactttttcagactgacaccgaagatacgaagaaaatgagtcatgaaacagatgtccttaaagtaaaatatgttggtgtatcagtaatacggtagtataatgtaaaaactacaagaatataaaaatcaagggagaatacctcccttgaccaatctagtttatttgcgttctagaccaaagaagatgcgagcatttagcacctgatgttcgaactgagctgctaaacctttgaaaattgtaatacggtatgcaccgtcaataagaatcatgttttcaattttaatagaatattcaccatcagcgtatgtttcaccttctggcgtttcaatatctacagtaaattcatttgaagactgatctaaagtagggttgcttgtaaccagcttgattacgttgttttctactttaaatttgatatgggtgtgacccaatacaccagccattttgcgtacgtcagcaatagtttgttctgagataacaaatgaaaggttgacgtctttagggtcaattacaatagctttgtcgcgatagctgatcattgacgcatcactgaagttataacgaactttggcgttgcttgatttgatttctacgtattctttaccgtctgtaaagttaagagaagcgcccgtcatagcagattggtttaacacctgcagtaaacggttcaattcatagacggcaaattcacgtggccatgtttcgtcaataacagcatgaagcatgataacgccgttttcagtggcagttttgatctcttgacctggtttaaacagtatagaagtgttaatttgtgcagctacttttaaaatagacagcgtgttcttagatagaacgatagatgatgcagacattggtttctccaaagaaacaaaataaacctttaacagtgaaactaaaaaggctgaatgattacgggataaatcatactacggttttattataatgtaataaatcaaataaaattgtttttcaatggatgaacttttagattcttaaatgaatgaacattaaaatataaaattagattctcaatccttgtctattacaagctcttagagtgtaaaaatcgtacaactggactactatatgggactagataaaaatatggtatgattgggatgtacagacggtataatcagtattatcaactctttcatttactttaagaattattaggagttgttttattgtaactgttataccgtctgtacactattatttagtcaattgatttttgttatatttctaaaataatttataagaggaacaggactcaggaaatgtaattttgaacctcgtaatactcgcttcgctcgcattactcgggtgcggagcacccactccgtttgatgtcgcaaacatagcatcacccctttctcataccattccatcaaataactcagacaattatattctcagcaatttaataatgatacccttgtcattaggaataattgataaaaatcaattacgatactcagtacccttgaggtattatcctcatacagtctcagataatcaattatgttaaatccatagtaaaacataatctcaatatcgtcattcaattctgactgtatctcatctttcacattgtgaaagcactcttgtttggcgtgaatgagcctttgaagatttcatctgaagaatatcaaaacaacagaatcaataatttaatctctcaaaccaagaatgaccctgatattcaattttatagatgcaaatatcagatattattgttgcattgtgactagaaattgaatatcacaacaataatatattccaaacatctctccctagacttcaacattcagagaatatagactttgtagattgggtctcagaccgtaggaacgctatattcaatcattcaatagaactatattgaagcccctcaacttatcagagcgttacctctaacagtgataaggatatacgcaaaagattgataaaatcaacgtctgccgtatatgggtcggatttgacaaatgaggcacaagtctcaccgactcaggaattataatttatttttagaacaaagtaaaggatgaaattttagacaataaaaaagccctgtagttgataaaattacagagcctttttattgagcggtgggattcaatctgaagattgatctcaaaatcccatcactcagatagctttaaagtgagattcttttaccctatttaagctatccagtttcacatacacggcaagtgatgtagattactattttaatacatctttgtatgataataaagtgttttttaataaaaatctaatgataaaatagttcaagatataatttattatagatttttccaattaagagattttgagaccaaatcaactaagtaaagacagttgttgaactaaacagtatatccggagtgtcacatgagtgataacaaactgaaaattgacgtcgtattagcagacgtaggcgaaagtggcgcagtcctatcagactttgtgacacaagatttacataatacagatatggccaaaaaggctgatttggttaatggtaaggttaatccaactcaacttcctgaattttctgagttgagcggtgtcaccgatgtgttttcccagttagaaaacaatttaagagaaaatatcaatatatcgctagctgaatctaaagagtacgctgactcaatagttagtgaagttatgtctaaaaaggctgatttagtgaatggtagagttcctaaagagcaagctacaaatttcaaagacattcctggcgcgaaagctgctatagatggattaaaacagtcattgggacaagagattattgggatcaatgatagtttagaaaataatttcgcaaaattaacaaatggtaaggtatctcagacggttttgcccaccgatctggtttattcagaccgtttaactcgtgaaatgaatgatgtaagaagcgctttacagaatacaataacgcctgaaagtccattttttggtaaaaagattggtgcgatcggggattcaatcacatggggatttacaccacgtaatgacccaaatggcggaactacaggagcgcaattgagtagctggcttgtacttgcagcacaaaaactaggaatgaccgcatttaattatggcattagtggttcaacattgggggcaaacagtgctggatcaaatgctcctttctcaagacgttatcaaaatatggcgaatgacttagatgttatttgcgtgatgggtggaacgaatgatgtgcgtaaaggtgtgccgatcggaacaatgtcagatcgtgtagatacaacttattacggtgcattgcacgttatttgtcagggtttaattgacaaatattacgttaatcaaggaactgaattgggcaagtcaaagcaaattattttgatgactccgattaagttacaagtcggctgaggtacagctttagatacagctatcgagccttatgtccaagctgtgaaagacgttggtgcttattactccatacctgttcttgatatgtacaatttatctggaatcactccacatattagtcaaacgatccaggggactgaaacaggttatacaaaaacatataatgtatacataactgacggtacacatccaacacagcagggtcatgaaaagatggcggctgctttttcaggttttttaaaaacacttaaatgacacgacagaccgtctccaaccttgatcttcggactggggttttatttagcaaaataggggatgaaatgacatcagatcaccctatgactgaggttgaattatagaggatatacatgttttctctgataacgagagttattgaagaatgaaatacataaatagttcaaatactgttctatctatgggccattgtcatgtctaaaacgttaaaacttgaaatctccgttgccgatctcggtcagaacggagtagttgtttctgaatttgcaactacagattatgttgcaaacgaattatctaaaaaagccactctaaatgacgaaggaaaggttgatccaagtctattaccagattatacttacatagatggtctagttgacaaattagatgacgttgaacaatcttatttggacgctgacgctgaaatcttaaacgcagcaaaattatactcagacaataagatatcggagaacaattctaagaaagccgatcttgttgaaggtaaagttccattcgaacaattgccattttccatgaatttcgagcagaatgtctcaactgaatttgaaaacgcgcgtcaagaattgagacaaaattctgaaagcatagtagctcaaatggatggtcttgtgggttctgctaattcttatacagatagtaaagttcttgaagaaagagaatttttgaacaatactatcaatgagtacaagggtgaagtcaatagtcagttgaataattttgataatgaaattggggtagtgaaaaatactcaagatgatattatcaagaatggagcttctctaccatacgatcctgatttggaatatgttgacggggcggttgttcttaaagatgaaagactacaaataaaacaaggtattaattggaaaattttaggaactaccattgcttcagaaatactcaactctgacggggaaaatttacaagagttgtgcgaaaaattgaagggtaatgtgatttttctcgaagactttggcggtacagagggcgcagattgcagcgacgctttacaatcttgctttgattatgtaaaaaataatagaaaatctcaaataaaaactaatttaaaaagtatttcgatatcaaaacgctgcgtatatcacgataatacaaatatagacttcaataattgtaaagtcaccttgattggtaatggacaaatcagtagcgccttgtataactcagatagaagtgattttataggattctctggcgcggaaatttattataaatatcaagccactaagactattaacaatcctaactctagaatctctattgatgtgccatctaaggcttctagtatttctgtttcggattcttctggatttactgtaggcgattatattcttattagtaacggctattgcgacatgtggcgcgtgatggaaaaatatcattctgaaggagcgcccgcagaaaaatctcaccaagactgggtgaagcctgaagttgatctctggcgttgtgaaatagctaggatcaagtctatttccggtaatacaataattttggaagatgaactcgaaaatgaatataaatctacaacaaagacgtatggattttttgcagatgaaaacaatagggcagatcacaacggttggaattatcctttcatagaaagattgggtggtgcctctaactgtactttcaaaaatttaataatgaaaaatgagggttcggctcccgtctctcttgttagttattgtggagtcaataataaagtcttagacagtgtttttacaggtaaggggcaaggcgcggactttatcacctgttttaacagccatattttgcgttcgtactcagaaacagagtcttttggcctatcaatacgcagaggttcgtatatgtgtattatggcaaatgcctcggctaaatacgttagtggagattgtccacttattatctgggagggtgcgcaacattgtattgcaaacaatattcaagtagatggaacaggcggaaatacaagccatgcaaaaataggattctattttaatacgtgctggaattgtataggaaacaatttcacgggtaaaaacttagattccgttgtaggggttttattctgtcgcggcaatattattgtaaataatatgaatggggtgaattgcggtgttttatttggagcttattcaactttcgatgttaaagcagatattggtacattaaaaggtgattacttggattctcctagtatttatgatgcgagtcttttctatgtagcagagtgtaataatattgaaatttctaaattaagatccttcgagaaatatagtgcaacaaaggatacaggacgcgctcatatttataaatcgttcgctgttgatttaaaagacattgacgcgccaaacgtgattttgtggaactcagtcgacgatgataaaaaatacgagtctttaaaattcaagatgaaaacgaaggattgcgtttttaaacttgcttatttaacacaaacttataataacggggaagctcaaacccgcagaacatattttcgggataccgattttatgcagtttgtggatataacatgtacgcataatacagagttgaatggcgtcgtaattaaaggacttgatgtaaatgttagtctgaaattaactatctcgagttatactagattgattgattgtattattgagaatgccacgtctggtattgatttcttaggtactggagcgtctggagcgcaaaactggacaagcttggtgtatatgcgaaatacagttgttaatgccccaactagatttttaaactataacgatcctagttatatgctttctataggctccggaagtccaaagggtaaaggtattcaatacatctctgttttaacaaattacccaaacttgagaactttcgctaatcctggaatggacggtaatagagttggatggtatatggtcgaagataccagacaggttgcaatagaatcattaacaactgctcaactctcggacaaagcgcatgcgatcaattctataggttctaaatggttgggtcgtgaggtttataattctactcttcagaagtttatgaaatcgaaaggcaatactacaacttcagcttgggtatcaactgacggtcagaatacaataactcctgtttgatttttaacataactcaagaagagttcagtcataaatagttcaatacctcaatctaatgaatgttgaactgacatgactcaatctctaaaaattgaatgcctagtgaccgatcttactctagagggtgaggtcgcgtcctttctagtgacaaaagagcaattaactgctggattaagcaaaaaggctgatctagttgatggtaagatcaaagaatcgaatcttcctgatttcattgctaatgggtttcaaacggtaaatcaaaaattatcaacattggaaacttccattactgatggaattcaaagcgcaaaggactacgctgaagcctacactgatgaaaaactgattcaaaaagctgatttagttgatggtaaagttcctgcgtctcaattgccatctatttcagaatatagtggtttaagtgaagccctaactggcgttctagatcaagcgcgtagagacatgattgagcgcacagatgaaattcaacgcgcgaaggctgatattggcgaagatggaaaagttgtcagagaacaacttccaaattacgacaagattcctggtttagaaacagttgttactgatttatatgactcgaaagctaatgtagatgacgtagaaacttcttttaaagaatcgcaagatcaattagatgcgcattctttacttctacaaagtttgggctcgattcttccgtataacccaagtaatacctacgcagaaggttacgtaactctgaaggatggttctcttcaacaactcgtttctgggaaatgggtaccatttaagattaatgctctaaacttggttgatctaagtggtgaatcgcaacagcaagtcaactacaacggcggttctaagtggcattataaagatggcggctatcaagagaatgagagagctgttttagacaatggcgacattgtgaaaagcatggttaactggaataagaatgaccctaatgtggatatgacggggtgggttaaaaccaattctacgagtcagatttttgatataagtggtttaactcagcaagagatcaatggcttgatcggaagcccgttaatgtttggtgctaaaggtgatggcgtttttgatgacactttggctattcaaaagtgtgcgaacaagtttagatacctggatatgcgaaatggtaattggaaaacaacaggtaaaattacgctaagagagggtactgttgttgatttaaggggggcgtacttaagtgcaaacaacgctaacgaacccatcttcgagtttgaaaatgcgggtgatggtttatctatactaggtggtggtgggtatgtttacggcacggcagactcatttttaaaatgctcaggaaaaacttatcaacccacgatgcagactgactatgcaagacaaatccgactagacggtgttcatgtttctggcaaaaatattgataagtttttagactttgataaagccgttaggcaggtcttcatgacaaaagtatttgcttacgtgaagaacggaatgttctgtaatggtaagtgcgtggaaattgagatttacggatcaattatatatggcgcaatggggactacagacctaacaacttacggaattaagtacagaagtccgggtaatggtcgttggtttaacgaagggttgcatattacagattcaacaattgataacttcggaacaactttcgacatcactgatatttacgcaatgagcgtaacaggtgggcatataggagctgctattcaaagcgatggaaataacgctccaggagctttagttgctaaattcgggcaaccaacatccacgttttgtcgcaatattaaattcgcaaatgtgaatatatacgggaatattgaatttgcccctgttggagggcttgactatcaagctaaattcacaggttgctcgtcagaaatgtgcaaagatgcaaatatttttattaaaaataacgcatcaagtattacagtgtctaaccacactttccactcctcgacaaacggtgttgctgtggtatgtcagtcgaataactttaatatttctgtaaatgatattacttgtgattcaacgtttgtcggtggtgtgcatgttcgcggtgcggttggtgataatgtttcagttcgagacattaattacgcaggttctggtgatgctgtctattgtgaaagaccgattttaatttcaggcgtacctgttgccacggcaggcatggcaaattataagcaaaaactggttaatgctccgcaggctacttatgctgtatcgcaaactatagcaacagtaagcgcagggtttgcgaaaggcgaaacaggagagattgttgttgagttgagttattttggtgctacaccgacaaaccgagcattaaaaattaatattcccgacggtttagttgttcctagcggctcaggctggagttctgagtttgttttattaaaatcggagtcaggtacgattagtgcaagaataccctactttgtcacaaagtctattgcgagtgctaatatttcgctcactaactttgagggtggcgcattaacactgggctatcacagctactttggattcaagagagactggtgataacccattcttgttggttattttgggagtaaaagtgctattataggtaaaactgatatagagcgaaacaatgcagatttttaaaaatttagaggagcatatcataatcgtgcttaacagcgttaagcgcgattatctactttctatcatcactacattcgcatgagaaagaattaagtctgctttcgcagacggtttaggacgtaaataatacattgaatcttctgagagaaaatacatgattatcaaaaaaggcgacaaaggcgctttagtggagcagattcaacagaatctactccaactggggtactacatcggaaagcgcgttgacggtgacttcggtcagaacacccataatgcagtcttacaatatcaaaaggataacaatctgaaaccttttgacggcattgtgggcgctactacagcaggccatttaggtatccaattgggtttgcaacctagtgatatgtaccaattaacggaacgctgcgcggagcgtttgaaaggtattcatcctgatttggttaaagtggtggaacgagctattcagcttactccagtacaatttaaggtgggtgaaggactcagaactctagctcgacaaaaagagttaatgaaacagggtgcgacacagactatgaatagccgtcatctgactggtcatgccgttgatctacatgcctacgttaacggtactgtttcatgggattggaaatactactatcagattgaagaggcagttaaacaggctgctaaagaattgaaagtgagtattgaatggggcggtgattggaagtcctttaaagatgggccacactggcaattaccttggaacaagtatccaaaataaataaaggggcgcaagcccctttattatggaggtcgaaatgacttgggtactattcaaagaattattattgaaacaattgcattttgtaattgcttctgtcttagcaattgtttttgccgtttgttgggtcaatacaaataatgatttcaagaactataaaatctcagttcttgaacaagaaggtgaagacaaagacaaaattatagagcgtcagactcaagttaataatttgactgaacgtctaatagaaaagcatactactctggagaacgactatgctgaaactctcaacgctatcaatactaatgttgctaacgctcgcaagctcattagtgggttgtcaacccaatcccaagaaatcagaaccagcgttgtatccagtgctgacacagcctacgcaaccgaagttgccacaacctacatcgacctctacggagaaattaaacaacatgctattgaatcatcagaacaggctgacaaagctacagcagaagccttcaaacaatatgaaagttgtaaactgatggttgatgaatataataagtttaaggatgattcaaatgaaaagaaagattgacaatacagatttgaatgaactactaattgaagcgattgaactattaaaaagtaaatagatgtattcttcagccctaagattgttcattcactagggcaaaacaaaggactctcatgagtccttttcttgttccttttgtattatcattgataatcttagttcaaattcccaaggtgctagagcgtatgacgtttctatcgactgattgaaatgttgctgaagcatttgggtatcttcgaaccgtcggagaaggtcttttccatcctctccgacaatcaaataaaaaaatctagaatagacgtaaactccacatcttgttcgtgattgcattcagggttttcgcatttgaacttagttttcaggctgatgtatggttgcgcaccaaagaagaatttagacatttcagcctttgtagacataggaacatctttacaccagtttttgaactcttcctcagtgaagtcatctagaccaaaaatctgttcaccttgagtgatagaatctaggtgcttgtacatacctgtggctgtgtcaatattggcaaaatctcggctgtattctccagcctgcatctggcgtaattttacgaaaattggaggattggcttcaatcacaaaggtgtctttatacccttctgttttaatcagctttacttgattcagatcgagaataacattcatctcgttgccgcaaggttttgttttaggtgtaccatctgattttagaatcggttcaccattttcattcttaatttccaattccttattacaacgtaatgtgataggaatctctgtgccgttgctcacttttcttaattcgatgaaaagtaattctgctatatatgacggtaatgatttagctcgccccttccatttaccaattagacattcatcctcaaggtttagaatggctgagaaaatttcagaaggttttttggagtccttgatcagtagcaggttattttccatgcccacgttgaatggtctgaagtcgaattcatactcttcaagtttaacgttatggacttggtacgggatagaaggaagcattttaaaattctccaaaaatatttgataaagtcagctttctaataaatagggttatgataggctttgggtgtctggatagaacgctattagacagttttataaatttacggtgaaatatatgacacgtgtttacatgggaatcgatattgatgattctcgagataatttgcttacagaacctgcgaaagaccttatcaaaaacttttatacgcgtagtggggaaacaatccaagaaacactagctcgtgcatgcgtagcttggtccgatgacgttcatcacgctcagcgcctttatgatgctaactccatgcttcatttcatgtggtcatctcctatgctgtctaatgctccggaactgaagcctgaagtgtggaaagctggtatgattaaccaatcattacctatttgggaacatgaagaagatttcaaccatattggctctcaaaatatttcgtgtttccttcctgctgtctctgatagcattccttctcttttgatgggcaaactcgaatcctctttattgtccgttattggcggcggcattggcaaatactttgatatacgttacacttctgataaatctcctggcccaattccctttatcatcgaagatgactcttctattctagcttggaaacaaggcaaagtacgtcgtggcgcagaagcgtgttggttgtctgctaatcatggttccgcaaaagaatttttgaagatccgtactccgacaggcgatgtaaaccgtaaagctgaaaacattcacattggattctctattgaagatgatctaatggaacgtattgaagatggcgctgaagaatatcacttgatcgatccgcactctaagcgcgttattgatacagtaaaccctacagactacttccgttctatgattgtagaacgcggacgtacaggtgaaccgtttatgtacttccgtggtaacgcaaaccgcgcattacctgaagcacagaaacgtctaggcttgtattcatatgcatccaacctttgcctagctggagacacaaaggtaattgttgcagacggtcgtaagcctattacaatcaaacagctggcgcgtgaatcatctggttccattccattcaaagtttggtcgggtcgcgtttcaaacggtaaagtgtatccagaagtcaaaacagctacggctaaattgacaggtcgcaaagaaatggtcactttgaccttccgttcaggtgaaactttaacctgtacgaaagatcatgaattgtttacaaccatcggaaaacgcgtaaaagcaaaagattctgttggtaagcaaattgattcgcgtctgttccgtcacggctctgtaaattttgacaattctgatcttacagttgcttctgttgagcaaaatgttgggacacaagatgtttactgcttatctgtagaagacaaccataacttctacgtcagcgtaggcggtaaaaatgatttcgatcatcgtacggcattagttcataactgttttgcgggcgaaacaattgtagctacggctgatggtcgaaatggggttgatattaagactctagctgaggaatctcagggcgttatggagtttccagtctatacatctaaagaaagagattctcgtaaaaagcacatctcgcactatgaagctccgtctatagcaatggcgactgcgttcaaaacaggaactaaaggtctagtgcgagtgactttaagtgacggcggcacattccgttgtacacctgatcataaattacgtcagatcaacggtgaacctgttgaggctcaacattctttaggtaaagagatgcaaacattcttctcttatgttacagagatggatggcgcgtatcgcacaattaattctttatctgatggaaacggaaaacagcatctcaagatgtggaaatttcataacggagatataccagagggtcatgtaattgatcatatcgatccatctgctaagattagagatggtatggacaacttgagatgtattcatcgttctgaagattctttccacggtcgagatggtaaagggcgcaagaatgcaacattctgtggtttgtctaatagtgagttgattaacttagctaaacaatggaagttagacaatggtgtagaagtattcaatggttggacttacgaaaaattaagactagagcaattacaactaggtgttaaactacctcaatcgttcagtaagtatcgttttgacggttcttggaaagcattctattcatacgtgaatgatgaagctgaatactttgaagaagatgattcaacttggattgataaatgtcgcattggttcgtctgaaattcgtaatcttgttgaaactgaattgcgcgcagacaataccgaaggtaagtttccaaaatataaacacaaaggtttatatgtaaccaatgttgaagttttggaagatgttgaagatgtctattgtctgaatgttcctggaacgcacaaattctatatcttgacaaaagattacggcgagaattttaataactctgctggtattctggtggataactgtaccgagataactttaccagcgaacgatgaccgtactccagtgtgttgcttgtcttcactaaatgccgagtatcacgatcaatgggcacctaccttagtaggcgacttagtgtgcgcattggacaatgctattgaacatttcattcgcactgcagactggattgcaaaccgctatgataatccacgtcaccgcgaattgattaaaatggctttgtacaaagtgatcaattcagcaactcaagaacgttctattggtctaggcgttatgggtcttcattacgctttccagaagcaagatataccatttgattctgaagaagcgcgtgtattcaacaaagcattattcaaacgtattaaatcagaagctgtagccgaaacacttaaattaggtgaagagcgcggtgaatgtccagatgcggctagttctaaagatcatccacacgtcgggcgtcgtaacatgcatttacttgctatcgcgcctaactctaacaatgctgatatgctaactacatcagccagctgtgaacctgaatacgctaacatctacactaaagaaacgcgtgttggtatctttgaacgccgcaacccttatttgaagaagtatcttcaagaactaggtcaagatactgaagaagtatggacaagcattgctcataatgaaggttcagtatctcatctagaattcatgagcgatcataaaaagcgcgtatttgctacagcttttgagatccctcaattgagcattgttcgtcttgctgctgaccgtcaacctgaaatctgtcaggctcaaagtctaaacttgttcttcaaaccaggaagtgatattaactatgtagtcgcatgtcacttctacgcttggaagtggggcttaaaatctctgtattactaccgtacgaaaccagaagtggagattgaaacattgtctcaagctctagagcgtaaagctctgaaagattctgttaagaaagaacagaaggctggcgagaatattgtttacggtacaaaagtctgccctaattgtactacagttaagcgttggttaagtgatatgggcgtggaattcaaatttgtcgatctacaacagattgaaaaaactgccgctgaagtaacaggcgacccttcttgccgttctgtgcctcaagtgttcttagatggcacttggatcggcggtcataaagaagtacaagccctttacaaagaacatctcgctcaagctgataatcttttctctgaagagaaagatgaagactgccctagctgccaaggataatattacatgtctattttaattgctgattctacaagcccattgttccaagattctctggtttaccgcccatttgtatacccttgggctcaggaaatgtaccaagatcacgaaaagatccactggattcctggcgaagcccaaatgcaagacgatcacgaagactgggcaaacaacttgacaaatgacgaaaaatcatatatccgtatgattttcaccatcttcacaactggtgacgttgttgtagctaaaaactaccgtgattgtttcttgaaacattacaagaacaatgaagtcagtaacggcttaatgtctattgcgtgccgcgaaggtatccatcaggaagcctacgcgttagcagccgaaatctttggtatggatgataccattttctctatgttccaagaatataaagaaatgcaagaccgtcttgaaggtatgttcgtgaacttggaatgcgatactgttgagcaattagcaatcaaccacgctcattctgttattgacgaaggcgttgcacttttcggtatgtttgttggtcttttacaattcatgcgccgtgaccgcgttgctttcaatggtaagcgcggtggacgtctaaaaggattctcgaagatcaatgcatggtctttgcgcgatgaaaccttgcatactgactacaacgttcaattgttccgccagctgtcgcatgaacatccaattgttgatgacaaactgaaacgcgacatttatgagatgaaccgccgtttcattactgttgaagataagtttttggaattagtgttcaagcatctgaaattggatacattcacaattgaagaaaataaggcgtacatgcgttttatgtcgaatcgccgtatgattcagatgggtctgaaaccagaatggccagaaatttctgaaaatccttgtgaatggattgagcctattctgacagaaaacaaagtgagttcattctttgaaactaaggtgagctcgtatcaagtatctggtgccattgtgggtgattatgacctgaagactatgttgtctttaggtgctaattacaaataatattgattttagagataatcgcaatacagggtttataatagccctgtattcacttaaatgaaagttttttaaattttggagtaacctctcatgacacagttaaacgtagacaaccaaaaagacatcgaacgcgctgaagcaattgaagtaaaggtggatgaaaccaatacatctcaagcagctgaagctcaacctgtcgcaatctcagaaacagttgaattaaaagacaatgttgacccaaatgatcctgtagaacttattaaagccatcttcattcagttcggtacagactacagtcaaacaatgcgcactcagcttgataacgtagaaggcgcgtatactttgacgtctttcggcaacccaaccttgcagtttatgtatcactctgatcctaaaggtgataaaattactgaagaagaaatggctgaaatcaaacgtactgtatacgtttctggcttcccagctgatgacgctgttggcattctagaaatggtattgaatatgcagaaacgtttatctgctaaaggctttgatattgcaattgcaggtgttatcgctccagttcaaacaggtgatggtcacaatgtaattatctctgattttgaacaggttatggctttgtctgctcgcgcggcattcctttctggacgcgcttctttaaaacgccttgaagatgctcaaaatgcagcgaaagcggcagaacaagcggtagctgaaactgagacagaaagttctgaagtagaaggcgctgaaacagctccgaaatcagaagaaaagacagaacaagagacacctaaagaagcctaaccataactttagataatcttaaaagaaagctgccgaaaggtggcttttttattgcccctttatagtacaactattttaaattgtattattgagggtgtaaaaatgtttgatgtatatgatccagagtctcctgaagagaaggtagaagaaaaggaatgcgataattgcgagcagctatataaagttgtacacgcaactaacgaaaagcctgtattctgtccattctgttctgaacccttggagtaataaatgaaagctggtatcgactattctatgagctgtcccgcaatctacattcagcgagatgatggtagcgagaaatggtatatcatccaagaagttctcaagctagtacaagaaacggaaaccatttctgtagaacgtcatccaaaatacaactccaaaattgaacgcgctatacacttagcaaaatgggctgtaggtatattgcgagctgaaggcgttgaagttgtgaatgtagaaggattctcaatgggtgcatctaatggtaatgcgattttcgatatcggcgctcacggtggtattttattctatgagattttcaaacataatattgctgtgaacttcatacctccttccacagccaagaagtcttttacaggcaacggttcagctaaaaaagaagatatgtgcgctaaattctttgagttgaaggggtggagattctctgatgttctagaggtgaaagaaaatgacagtcctgaaaatgacttggtcgatgcttacgcaattgcccactgtgatgtattacttgatccaaaggcgaaagctaaaatgttcatgtgaggaacgtattgtgaatgatagtaaaatgattgttgaccgttatacatcatttaaagaaatgatggattggatcaacgcggttgtatccccttctgaacgttgcgcaagagcgaattacgtatttcaaaaagcgccgtacttgaagcaaacgatggagatgtatttcaacgacgctccgcacaaatttagtaagttgtctgaaagtattgatgacatcgtttctaaaatcagaccactaggtatcaaagaccatgctaattttgggcttttcgtacgtgatggatacatgtggggagacgattctccgctaaacgattataacttgaaaagcaaattatcaggacttttagattatactattccatctgatgtagaatttatttctaaatttgtgaaattgtcagagattgaagaatttactgaattcggtctagacacttataatatgttagtggggaaataaatgcctgtttattcatacgcttgtcgaagttgtgataacaaattcgatgaaattaaaaaggttgatgaacgcaatactcctactgattcaccttgtgtcagctgcggcggtgagctatatattcatatagggggtggtttaggttatgttgataacactggacgcctgaatcagaagaaagtgcctcaagacttccaaaatcttgtttctggaataatgaagacgcacaaacaggagtataagggatgattggtgaaacggttgaaaatccttttggttgcgctgaagctttttcaaaagcagttgaagatttagtctgggctgaagatattaccctttctgaagcgctttcattaattgtagaagaacatcagattgactacgataaagtaaaaactatgattagtcctgcattaatcagtaaaattgaagctgaagttgtagaacagcgcgtcattcgcaaggagcgctctaccgtttcgctagataatcttttttaatttgattttttaagaaattcagatgaggggtatataatatctctcactgaagaggtgagataaactcactagggcaaaagtccaaacgttggtcagccgtcaaccatgaataaccagaatgacggaaatagaaggaagaaagaaatgcctgtagattttggtgctttacgcaaacaaggttctaacaacatggctcaactccaaaaaactttggagaaaactgagaaaaacaactacgatgatccacgcgtctggaaatatgcgcgcgacgaaaaaacaaacgagtctgtttgtgttattcgtttcttgcctattccagctgtagatatggaaggcgtagaagctggtacattcattgctgaagacttaactcctgttgttaaagttttacgtcatcagtttaaaggtacaggcggaaaatggttcaacttcaactctcctcaaacgtttggtgaagaagatccagttcgtgaatggtcaggtccgaagtggggcgatctcaagaaattgggtgaagggcatccggactatgaacgccgtaagaaggaattgttgaattacattccatctgaagactattacgcgaacgttcttgttatctcggacagcgtaaatccagaaaacaacggtaaagtgaagttgtttaaatttggtcgcgcggtgcgtaaattacttgatgaagcgaatgagcctaaatttggcgcagatccgtttgatccgtttgacgcattcgcaggtcgcaaccttcatttgaagttgacatttgaaaaacgtaactttggtggtcgtgaatcttgggttccagactttaaacattgtgaatgggattcgaaatcaactcctgtttctgctgatgaagctgaaattgaaaaatactggcgcgaatcgttctcgttgcttgaattcttagaccgtaaaaatttcccttcatatgatgttatgaagaaagaattttgtaaagtcatgaatatggacgaaaactacaatccagtagctggtactcaagcgcataatattagcggtgcggcatctgctccagccccgagtgcaggagcttctaatccaaatacagctaacacagctaattctttggatgctgcttctgaatcgccagctgccgatactcctgcttctactccagctgcggcttctccgtcatctgctgatgacttagattctttggctgatttgttgcgcgactaaaggcgttaagaaaggtggcttcggtcacctttcttttttcttacacttttcgttgtgatattgttttatattgctgtttgttgttacagttccgcaataaatacatttgcttctaggttgatttttgactttttcctttattctagcaagttgttcttcagtgaaagttttgcctttgaatggcgagtaatattcgccagatagtctactttctgacatctttttcttttcttctacagttctaggaatacctttattccaagcttttgaacctagcatagattcggacatctttttcctttcttctgacgttctcggaatgcctttattccaaggcgttcttcctttattggcatctgatattcttcttttagcttcatccggcattgaaccatcttgaccaccttctctaacgttatacgtatcctttctcataacatattctttattgactatttttcgttctgcttcaagcgcttgttcgcgtgtatcaaaggtttgaataatagacttcttgaagttgttgttcccttctttagatatagcttcctttaacgccacgccagaccccatatatttatctctttcgatggattttgaacaacttctcacgccaacataactcataccgtttctgatatttgttatttcgtaaatataatgattcatttgatttcctttatctttacatcgatacgttataatatctataacgtttagaggtgtaaataattatgttcaaaaaacctgtgatttcaatagacgtagacatgacggtggtagatgttctacaaccttgggttcaatggatgggcgtcaatcatccagaagattatattcgtccagcatatgaatcattagatggtcactacaatcatcacctagctgatctttatggcgttgatcctgatcagattaatgatttttggaaaaagaatgacttgtacgacaatcttcaaccaatggacggttctgttgaagcgtacaaaaaattaaaagagcattttgacgtttatttcttgacaatttgttatgctggacataaagattcaaaacaatcattcttacaacgccactttggtgaagatattgaaatgattgattctatgagttgtaagagtcattatgacttcaatatgctcattgatgataatccaaaagttcttatgggagtgcataacgcgcctcagaattttgagcataaaaaatgggggcgtcatctagttcaactaaaccaaatcacaccccaaaatatgaagttgttatctaatggcgatgtctctagtagtatcattcaaacgcaacattggaatgatatagatagcatcattgcgcgttgtttcaacgtagcgctttgcgcctaataattcttcgaacatgagattgtacagctgggtcagctgaaacaggtttgagccagcgttctgtacgtaaaaagcacgccacgatccaatcctctggtctgatctcaacaacatgtgatctaaaacggtctgcgcgatacgcgtggactgctaggtctgcaattttactacctgcaactgataatattgcttcccattctaatctcagtcttgttctctcagatattccttttgtattcttcagcttcataagtcctttatacagcgtagccctttgagctggcgtaaggtaatggacattcaaccccagtagaatagtcttgccttccttactcttaaatgagttaaagaagaaacacatcggaaagcggtcatacatgccgtgttctccgccagcaccttccgcttcatactcaaatgtatacaatgaccctgcgcttagtctagctgtttgtttagctgttcttttcttgtagtctgaatcagcgtcaatgattttaccagcttgtacctttaaatcctttgcaatacgtttttggaaccattctactgcttgtttactgtattgtttttcttgagtcgcgtttgccttgaagaacttgtcccagtaacgttctatagcttttggactcatatattcatcaggcatcttgatatactctcaaattgtatgtcataaatatttatgtattgagaggtatataacatgagtgcgcaacttcaacaatttgtgaatctagtaaagaccaaggatgttcaacgtccgaatagatacgacgttgaaattgcaatccctgaatctgtaataacaaaattcaatcaagaatacggtagtgagcctattaattccagattgacccttatggccaaagatacaaaccttcctggatatactgcagctgcgtcagaaattcacgtgggatataccgcctacgtggcgtacgaaaaggctaatggcgatttcagtatcacgttcatgtgcgctggcgatatgatggaacataagctattcaccatttggaaggatagcatattcaaggaagaccatagcgtagcattctatgatgattacatcacagacagaatgagcgttaagttgaaaacaagggacgataaggtatcaagacattgcgatattacagaagcgtggcctgctacaattactgacgtgacggtcaatagagatgaacgtgacacatatggaacgttccaagtgacgttcaaatatcgcaagctcaagtctatggataaacaatctgtgacaagtggagatgttcaagcatgagttatggtaaatggcatcaaggtaaatatcaacttataaatccaaagaaatacatcggtgacccaacaaacgtcgtttttagaagttcgtgggaacgcatcctaatgaaattcttggatgaaaatgaaaacatcttgaaatggcagtccgaagaacaagaaataccttactggagtcctgtcgattccagaatgcacagatatattttagattttgcattctggagtaatacagctgacggtattaaaaagttcatggttgaagtgaaaccaaagaaagaaactgttgaacctaaacgtggcccaagtgaaagtgaatatacattccaagaacgttgtaagacatggttgatcaataaggctaaatggactgctgcaattgaagtcgctaaacaagatggatctcagttcattattctaactgaagatcatattatgcctaaccacaacgctatcaaggcgtataaacatccaaaaccgaaggtgaagacaaatggcaacgtcagtcgcagagaaactgtcaaaaaagctaaacaacggcaatagtacagagaagaaatcgaacctacagcagcaactgattatgcccattgatctaattgatcgggatgatcatcctggattggttgttatgtttgagctcaatgccattgattacactaaagaagcatccactactggcggcggtaaactgaagtccaaaaacacttcaggcgtagagaatactgaagtggaaatgcagaagaaaggaaacgccaccaatgctgcttctgtgcgttcatcacttccgggatacagaaaaacaggagaatctatcgttcttcctatgcctgatcagtggatcgaaaacttaaacatcgcttggacgcaatttgaatttggtggtctggctagaaatgctgaactcatccagtctgtagttcaaggcgattacggcggtgctggacaacagggtgcggcagctgccccaggagttttggctaaggttgctggtaaagggggtggtaatctagctaaggtcgctgacggcgctagacgttatgtagaacttatggctgggatcaaggcaaatgactttactgagatgttattctcgaatgtaaacaaccgtatgcttccgttccaatttacattcacgcctagaaatcaaaaagaagctgaatcgatgcaacaaattattcatcggttcaagcacgcagctttacctgagttatgggtaaatggcactgataacggttcatacttcagagcgccgtatacattcgacatcagctttgttgattgtaaaacaggtaatcgttcaaaatattggacaaaattacaaacagctggtctgataaacatttcagtgaacagaacgcctaacggtggtttttctgtcatcaagaccgcagacggtgattttgtacctcaatctgtaactatagaattacaattcactgagttgttgagacttgtgaaagatgaaaacaacgaccctaatgaaagctactgatccatatagagtagtctggactgacgaaaaagtatacaaaagcccttgtctaatacaggggctttttaccgaattttaagaaatactgatcatgaacactaagtagttttaataattgaactattgagaagaatggcgatgatatctcgttttttcggacaggcagaatacaataagcaaggtcagattgtagcgatccctgatctgactaagcatgttgctattcctgatgaatacaaaaataattcaaaatattggattcgatataccttaaaagactcagatacgctttcaggtctatctaaatcagtttatggctctcatcattattggtttctcatttctcttctcaatgatatggttaacgtagatgacagttggccaattcctgaatatggtttcagagaatggtttaagaaaacattccctgagaacgagtatagtgatgtatatcaatgggttgattcaaaaggcaatgtccatactccagaagcctatgttgatctaggtgaggtgtcaagtgttaaagaagcgattcaaaaatacagtttgacgtctgtttcgtacttcactcatttttacaatttgaatgaagagaaaagaaagattaaactacttttgcctgagtacgtttccaatataacaactcagttggagactatgttcagatgagcgacttcatacctttagacacatttcgaatagagtgtatcaatctttacagagaaggaacgcctgaagaagaaaagtttgacataagagattttgcatacgagattgctatacatgaggatatgttttctcctaccatttttgctcatattgtaatgaatgacttaatcaatcttccggaatctttcccattagtgggcggtgagttcgtagacctacgtttcaaaacgccttctcagcccgatgttcaaacgttgacgctttgcgtggctaaggtgggtgagcgtttacagactgaccaaaccaataaacaggtatacactctacaccttatcactcaagaccgttatctagactttaagataaatctatcaaaggcttatgatggaacgtatgacaacattgttgattcgttactgaaagatttgggtacgactacatctctagagaaggacacagcaaattataatcagaccttcatatgtccatattggtcacctttaaaggcttgtcagtgggttgcttctagggcatacggcgttaaaactgatccatttatgttttgggaaacgatggacggttataatttcaaatcgttgtacaacgtctacgctcaagagccatacacgaaagtctttattgaaccatctagggttgatactgacggagacaaggcttggagaagagtacaaaagtgggaatataaagcttcagcagatagaatgcgacaactttctaattcagcttggggcgtgaatgtcattcttctcaattcagagtctagaagcattgagaaacaaacatttgactatagaacgctatctgaagcagaagattttgctaagatagatcagtttccgctgtatccagaagcggatagcaagatgtttcacgttagctttatgtacaccagagcagacaaaagtcatgagggtgaagtctatagaaacatgatgatgtctatgttagacttttatcgcatgaagatagtagttcctggcgatagtgggtatagagctggacagattatcgaaatggatgtacccgacttctcatcttctgctgttcagaatgaaactctgacgtctgggcgttggcttattgtatctctatgccaccttatccgacaaaacaattacagttgctcactagaactctgtaaggatagctatgcagctgatgtcagagctgaaataggtttaccagaggtaactgaatccgataaaaaggttgtagaaggtaaagaagcccagaagcccgagaacaaaccagctgataaattaattccggaagctaaatcatgagcgaaaatcaaagagaaacgcctacagaattggctaataaacgtctacaagagcttgagacagctaaggcgactaaagaccttaatgacgctatggctaatctgtctaagaatggtaaaaatgatctcagcaaagaagatatgttcaaagctatgcacgagatgactagacagatcaagctaaatgaatcaaagactattgatacagtgacatctgctgctatacagacggctttggacgtttctatgcaaatgaagtctttagatgaccctagactcagacgtcaggctatgatcactctgaagacaatcaatgataacataaaattgtctgaagactcatcgaagaataatgtagatgaagcagctgttaaacgtaatcttgttgtagctatagacaacctgaagactgaaagtctcatgtctcgtatagcagataagatgagtcagaagcaaggtgtagtgggtgacgcaatggataccgctgggccaatatttggtcttatgaaagatgcattgaaaaaggtatctagcgtaattgtatccaagaatacgaaggacgaaacgccagcattcttagatcatcagattgagcgcaaacctgttcaggatacgcccaaaccagttgctttaccgcctgtacaggttactgagaaaactggtaatgagattacagtcccaactattgcgacagcagcccctactgttatcgtacacatggatgatctgaagtccgacattcatgctgtcaaagaagctgatgagagtattctagagcatatcaaggatatctcggctgtaatgaaagagcaactcattatggctcaagatcagagtgaacgcgacaacgctcatcgtatggaagccaatttcagaaagttcaaagtaaagggtaacagtaacacaaatcaacaaccaactcagacgcagccagaaggtagttcaggtgggttactagattttataactccattactgtttggaccaaaaggtctgatgaaagttcttatgcggttcatcataggcgcagggggcatgattctatctatgctcaatccaaagaacatttggaagtttgcaaaaggcgtagtgactgtcgtaactgatctagctggtaagattttcggaaaattgggcggattgttcgatgatttgggcggtgtattgggtaagattggtaaaatcggtaaattagccaaaggaataccgtatattgggcaaattatagctgtagttgtaggtatattcgactttttcagttccttcatgaatgcagacgaagttctgggcaaagctaaggaagctattacactttgggacagagtgagcgcaggtataggtggtttcgtcggaggtatagttggtatagcagattctatactaggtctgtttgatattgaaacagacatgggcggttttgttaaagaaaaggttgctggtctactttctaatattccaaacttgattgtagctagattcgagatgctattagacctaaaagatgtcattgaacagaagatagattctggtatctccgcaattaagggcggatttcaagtgtttatggatattcttgatgatatttccaacttcatctctaatcttggcccaactctcaagaagatgtttactgaaaaggctagacagattcttaatgtacttcctggcgatctaggtgataagctgcttccaactgaagaaaaaccaaagcaaaaggaagatgaacagaagcctgagaccaagaagcctgaagagcagaattttgtccaaagagcgctatctaatcttccatctttgtctgatatatttggtaagaaggaagaaaaggctaaggttgagaaggaagctgctattgttgcagcgcaaaacaactcgactactaacgttaacaatgttagtaatacaagctcctctaacactaacatctatcagtctatgcctatcgcaacgagaccatcagatgattccttcagggttgttatttgatttttataatatttttaaaactaaggtatgatttcgatcataccttttttatctacagtaggaacaaaagatgtcttttacaattcgagatattctagcaatgtctatgggcgctcaagtagctcgtatgaattctcttttcacgcgtaagttgggcgacgctccttgcggttctaaaaatgcatacaaccaacacgcctattcaaaatcagaatgggatgaaaccgttgaagctatggaacacggctatgtttctgatgaatttcgtgacggtatcgcagaccttttcactactgtaattggaagtctttggcgcgctggtgatgaagattctacaatttttcatctacactccattccatctcatggcggcgaacaacatttcaattcagaagcgtttaacaactataaaacagaaattgatcgtctgattgatgatttaagttgtttcgtgtctcttcagggtgtcatcaaagtatctaataagacatttgttgaaaaattacgcgaattgttgaatgttctacttcaatttgccaaccttgtaggtatcgatccgcatgctgacttgtctcaagtcacattggccaacctttctaaactatgtcaaggtcaagatgctcttgaacgcacatatgaaaaatacaaagaaattggcgttgagctttatcatgaagaagttgaaccaaatatctttgctgtgttctcgaaaggcgatcaagtcgggacagacggcaaaacgtacgatgccgataagttcttgaaatcagcagactggcatgaaccgtactttccatctatgactaaagactcagcggcagcggcagcatcgatggcagtggctcaagttcagactacgcctaaagcccagcttgtagatgaaaagggtcaacctcttatttgattttttggtattttgtaaaaatactttataataactggagatcactactgatctcctttttataggaagacaagcgtatgcgctataaagccccttcaggatggacgtttattgaccacgagacattaagtctatgggatacatccatcattgcaatgactggcgtttgttatgtatcagacgcagaactaaaacagggtcatctagacggaacagatgaattgaccctagaatctttaatcgaacgcgctaaagcatggaagtttaacattgatgagcaagtgcgtctaggtcgaggtgtttctaagagcgctatgcaattctggaaagatcaaccagaatctgttcgtgaaatgattatcaaacccaaagaaggtgatatttccgtattggaaatgttcaatctacttcaggcgtatcatattcagtacggaacgcgcctagatcaaacgcgccttattgatcgcaacaacttcgatattacaaaattacaacatttgtatatggtaacaatgggtaatgataaatcccagactaaagtgcctttgccgtataatccatacgccacttgggaatgttctgtaatgttcaaattgtttactggttgtcgttacgctaaactcaagcccgaagacgttaaatgcgaacaatttgtttaccatcacccagctcatgatgccgcgctagatgcgtaccgtttcatgctcacattctcaggtaatgactcaatcgaaggataagatatgcttaacgaagctgaagaatacgtaaatccatataacttgttgcgctctggttctactccagaaaatgaaatgttgctaacattagccaaaaaacatttcccatttgacaccccaaatcctggacaattacaagctatcgtttgggtgtgttcgcaatttttgaagggtgaaaaacacgttgtactacaagcgcctacaggcatcggtaaatctgtaattgctactaccgtggacgctgtattgaaagacctagctaaaggcttccgtaccactattattactgctactattggtctacaacaacagtatattgagacagataaacgcatttatgatctacgaggacgtaccaattataaatgtccgttcaataaaggaccatataacagcggtcagtgtcgaacaatgcgtcgtaacggcggttgcgtcgcgaaacgcgaatgtccatacgtcattaagcgcactaaatggtgtaatgaagcagaactgcgctcttcaaatttcagcttccaaattgaagcatgtgattctttgtgcatgactgaagaaacaaaaacgcaacttatggttattgatgaatgtcatgagattgatgatgccatcgtagaccatacgtctattacaatcattccgggacaataccgttttactgaagaattaggcttccctcaattctaccaaggtatggttgaattacatgcttccttagaagcgtattctaaacaagaatacttcgatacgccagagcaactatcagcagattgtaatgttctcagtgaaatatcagaagatatccaagaaacattagatggtatgatttcagacaaagatagtggattgtctgactccaaagttgagcgctatggagatatgctggagctgatgcaacaggttggagataaactgactctatttggtaaagcatctgcaggcgctaaatggattgtaaatgaatacaaaaatgattcagaaagtcagttagccattgtcaaacctattttcgcgtggcaggttgctgactatgccttattccgcaaagctgatatttttcttcatatgtctgcaactatttgcggtattgatgaatatgcgtccactctagggctttctaaatttgtgtacggcgaattagacaatccaattccattgaataaccgtaaaatctattacattcctaagttcaaggttaatgctggtttcaaagactttgaaaagatgggtacgttcatgggtaaaattattgaacgtcacgcgcctagtaatggattgatccattgtgtgtcttacgccttgggtaataacatctttcagcaccttccgaaaaagggtggcgcttttattggacgagatcgaaatgaaattgttgatgagatgtctaatcacaattcaggtcgagtgtgtatcagtccgtctattgtaaagggctatgacttcaagggtgacatgagtcgttttcaagttattgctaaagtcccatacggttatctcggtgaccctttgatcaaactgaattcagaatatcgttcaggttggtatgcgcgtaaagccattctacaaatcgttcaggctgcagggcgttctgttcgcggcgtggatgatcatgccacaacgtatattacagatagctcttttgggcgtttacttcacgacaaccgtgaaatttttcctgactggttcttggaatcaattgtagaggtgaaaatgtaatgggcggactattcacagaagatgaacgcaaattgtataacgctgaacaagatgttatacaagctacaaagaaggcaatggctaccgctatggaagttggtcagggtgtggattcaactaaattgaaaaacgaacttcagcaaaatttagttcatactcaagagggctatgaatttttaaaggttttgatgaaccgtcacagctccggattcaataaattggatcacggcacatacagttatatttctagcgctatgcgagaattggaaaaagttgaaggactcaagaaatgaagttgaaaaatatttgtaagcaatacaacgtaaaagcatctaccattgctacagtagtattcaatcatgccaccttacagatcacgcctgaaatgaaacaggtacaagtgcagaatggtgacctttatatgcgtcaacattatggcaatatgaagtttggagcgtggaagtgtgtaactaaagtagacgctgctttggaagctgagctcgaagcaagtattgaacgcgtagcctgatacataagaaggggcatagccccttctttttggagtgtgtaatgacaagaaaatttgaaatctcgaaaaccatctctaagaactctgatattgagattctacctgagattaaacaaactggtaatgttttgataaacattcgtggttccaacggttcaggtaaatcttacaccgtccgcaaaatcattgagaattttgggacaggagttttgaaaacgattgatgttccttcagaaaagcatggaacaattacatttccatattatgaatacgatgacttctttttgttaggttcatatgaacgcgactgcggtggtctagatgtaatacgcgacttctatgttattgcgcctatggtgaaagaccttattcgtacaaaatctgttgtcatggaaggtattttatggtcttccgtattttcggcaatgtaccgtctagatcgagagcttcgagctatgggacatgtatctgtttggtgtgggtttaatcatccttgtgaacttcatctggagcgcattatggaacgtcgtgcacgcgctggaaagtttgaaccaatgcagatcaaacatgtactagagaagattaagtccactgaaaagggtctgaaccacgccatcttctacggcgctaatgttttgattggtccaactgagtatcttgcgcctatagtggctgatattctgaagaacaactctattgaaggcgttgactttgaagacaactatcattttgttccagacttagagaaatggaatgaaatctaccgttcgaacagagagtataaatattctccgtctgatgaattagtaaagaaatattcaacaacgaattcattgctggagttattctagtgcaactttgtcttcctgagtttgttatagaccgtttagttgataagggttataaactggacttatctgaatggaatacaagagaagatgaatacgatgtattcctaagattcaagaacagttttacaggcgttactcataaattaacaggtcgagttaaagacaattctttcattttgaaaagtgttaaggtgattaagtgaattttcctagaataatcagacctctacaactattcaacattgcgaaaaattctaactgtttacgccttcaggtgggggcagttggtaaggactccaagggtaatatatacgctggatggaatcacatggaagatggttctccatgcgaaataaatcctgaactttctaagattgacgttgttcacggtgaaattcatgttctgcgacaatcggatgatattgtatctattcatgtaacacattctccatgcttggaatgcgcaaaacatatagttgaacgcggtgttacatatgtagaatacattgaagggtatcgtctttcagacggtattgaatacttgaaacgtatgggcgttgaagttgtgcaaattaaagcttcagcgcctttacaaaagcctataattctcaaggatatttaaaatgcaaacacgtatgccacctcacattgaagtaatgattcgcaagaatccaaatgctgtattaactccgcgcggcatcgttgcgaagcgtacagaccgcattgatgaacttttaatgggtcttcctaacgcagacaaaatctattcggagcactttgatgaaatccgttcagaacaatcagccaatcatgaattggttaaacaaaaaacaactgaagcagcaccagtgggttcaaaagatgatggtgttgtacctgatactgaaggtgaatccaaaatggagttaccagaaaaggaacaacaagttactgcatccgaacaaacagatgttgaagctgttcaagatacaaactctaccgaaaaggaactagcgttcccaaatactgatgatgaacgcacggcgcaagaagtgtcagatcaaattgtagaacaaaatggctatactgatccgttcgatgctgcttttgcagaagcaaaattgaatccaaagtataaagacctttctgatgatgaaattaagaaacgcgtccaaatgtctattgccgcgaaagaacgcgctgctaaaaagaaagcagaagctgaagcagcaaaatgatgtaacagtaaatcttaaaatttagattttaagccctgtctagtacagggctttttaataccaaaatagtgggttccagactaccgtttggaattcgggaaatattcctttactttatcttgaatattgattataattctaaatcataaagttcaatatcactcaacggagtaaagacaatgtcttctaatatcatcattgaaaatttttctgaaaatgtttacatgacatctaaagacgcgtcacttagtggtcttcctgctctaaattacatcaaaggcgatcgttggatctatgttgcttttgataagtctgttgattcagctggtgggttatacgaagttaatgaatcaagcattgaagcttgtgatggcggcgatgctattcattttgaaactttatctgaagtgtctgaatatataaaatcatgctactatccatttacaggtgagatgattgaagcgtacaaacaaatgcttatcaaaatggatttatttggaccacacttaaaataatactaggggcgtttgccccttttattttgcctgattgataaatatctcaatagagaatttatcaatcgggcttttttatgtctcaaatatcaagaccgttgtattgcggcgattatgtgggcatcgttgaagacctaatggatccaactgaaacaggtcgggtgcgcgtacgcatatttggtatacattcattgaacactgctgaggttccaaaagaaaagcttccttggtgtacgattgcggttccagccactaatccatttgtatcaggtctaggagcagctccattagggcttctggaaggttctatggttaggctcagaccaaagagcgagaacgattatcaagagtgggaagttcaattcaccatagctggtcaacgccaaaatctcacagatacggctatgccttttccaaatggtacagatcatgatgttaacccattagctagaggtattgtaacgtcttcggatgtttatgcagctaaagacgctaatgcaattgaaggtgacattgacggctctaccaacagcaatgaaacccaaattagccctgaagagttagcgaactacccttggttgaaagtggccattggtgaactgggcaagaatgagcgagatcatcctaatcagatcagacaataccacgctaaaggtggcggttcatctaaatggggaggtgaaactccatggtgcgcttcctttattggttggtgtttgaaccaagttggtatcaaaggttctggttcagcagcagcgcgttcatacgtgaaatacggtaaaaatgttctaaaccaaaaccccatacctcctggatcaatatgtgtcattgctggtaatcgtggcgctagttctgggcacgtattcttctgtgaatatgaaaagaatggacgcatctacgcagtgggtggtaaccagtcttcaaagaataaggcttctcaaaacggcggtgaggtcacaagaacatcatatgctagatcgtctttgattggagcttcattcccagatactgctaacttgaggaaaaaataatgagtgcggaaaatattcctgttgaattaacagtagaccatctacaaaagattttcggtctgagtaatgcatcagattggcattcagtattatggccatatatgaagaaatatgatataacaacaacttatcgtatttccatgttttttggtaacttgtctgttgagtctaataactttaaaacacttgttgagaatctgaactattcggctgaagggttgagaaatacatttccatctaagtttgcaaaggtatcagcggagtcatatgctagaaatccacaaaagattgctaactacgtttatgctaacaaaaatggtaacggctcagaagcttcgggtgacggttggagataccgtggacaagggcttatacagttaacaggtaaaggtaacgtagcagcctttcatcgtaaagagccatcattcaattctctgagcaaccctgaagtattgcagcaaaaacctgccgcttcagcttcagcttgttggttctggagtgacaaaggtttgaaccgtcaggctgattcaggaaactgggctgtttgcgtgaaacgcattaatgctgctagtcttcatatggaagatcgtacgtcaatgtataaacgcgcattgagcgttttattgggaaaggaagttgaggagttgcctgagtcatcaggtagctctactgacgcctcaactccagattctagaatatctggggttactgaaccaagaggtaaggcagctcctcaatatccgcataataaagtctatcaatcaccatctggtcattgtattgaggtggatgatactcctggcgctgaaaggattatgatatttcatatgtcaggttcttacattgaaatgcatatggacggtgatgttgtgataaaggctcataaagacctttatttgcttgcgggtggttcagttcagaaacttcagaaggaagaccaagcagaaatcgtcgaggggcaatcttatgagaaggtcggtcaaaaggttatgtctaatgatcagatgactgtagaatcgtcagatttgcaggtgaacgcgccttcagccttttctaagcctatggacgcaaaagagattaattccacggatgttaccgcagctgaattaacttgtaccgcgcctgtcgctcaactcaatgctgctactgcagaggttgcgaagtttgctaacgcatttgctcctggttctccagctggtatgtctctagctaatctgtctcagtctattcagatagaaactgatcaagacggcggaagtaaacagatcaaaccagacgcgcctttgaacgttgctaacggtatgttcattcctagacttgacgaaatgccttctcaatatagtggtttctttgtttacaatgatttaccctatcttggaatgggcactcaaataataccactcggcgcgcaacaaaatactggagatagctctaatggctgattttgatcctaattttctcatacacccgcagactagagatttgaacctgttgcgcggtaaaaaggctgtattccaagccttcaagaccctagtattatctgggattgaagattttattatgcgcagcgatatccacggtggaaatatctatcagaccttgtttgataatgataacgctttgttgagagcgcagataacatctaagattgaggagattgctagaattgacgaaccaagaattgttattaaagcaatagacttcgatggttcaaaacatcaccatctcattatcaagatcaagtatctgtacgaaaatgagctggagatgtctgaagattctattaagattgtacgcacatcttaatattgagggcgttcaataacgccgttaaacaataccactctattgccacctttctttgcattgatataaaagtgaaagaaaggcggcggatttacaagccccattgtttcgatagggtgctcattcaacagttcagattctgttaattgaaagattaatgttctgggtaaagctgcgtcaaaatccaaaggcttagaataatacggctgccctattttggaaagtgaaagatgtagttgaatatcttcatattcatcaaaacgataactcaacttagagacagggtcagttccagatattgtttcatttatatccgcgcccatctgagttgtgattttaatacggttatcaatgacgttcatgattaaatgttcctctgtttaatatacttacaatgtaaataaagtaaatcacgcgcattagagcatttaacatgactgatatctctttaccatccatatatcctaaaggagcaatgtccaagtctttaattggtcttgaacagctattgtttgggcgtaactttgggtgctctacagaagaaatacaagtgagcttttcgggttctgtttgtaatattgttttggattataagcaaacaatctctggactttattttgtcagcgttgaggatatagacggcggttcactccgttgtctcatttctgcgagcgctaagcaatctgcatttgaaggttctattgaatatcaaaatacagtacaattattgagtctaacgaccgtcaataacatgtccttagaatatttggacataaaggcttcatctacaacggacgttggtcttttaatgagcatagctgggacagggtttgaatcttatcctgtgaaggttaaagtaactcaggttggttcaagttctaaggtgagaccatcgtcatcttttgcagactacaccattgatatcaagaccacagtaactggtattctatcgacgaaaaaggtcattgatgatagcgaaggggtgaacgttaatctcagcgacgttaatgaaaattccataatactttgtaacccagctagtcaatcgtctagacctaataaatctggtataggaatgtgcacaacattcatctcagatgtatttaaagtacaattgttttggtctgatgatggtaagatgtatcttcgttctggaaattcatcttgggttggggttgataatgattctacatatccgaaagttgtctatagtggaatattctttgtatcttcagatggggtagatgagactataatcccagttaacggcatcgacctgcgtcaattgagatattctataacattcacgcctcaatatgacgttgtgagcctgacagatgatattcaaatatctagtagagcagaaaatggatttggggtaaaaactgtaaaaccatgggctggtgtaatttcttattccatcacagagacacgcgcagctgaatcagaccctgtatttaatttaacttgcgggcgtttggatggtcaggatcttcgaccagctacttctgttgaagaagggcagacagctagattcgtactttcgacacaaaatgtagcagatggcacttctgttccatttactattcaaggcgttgatgagtctgatgtaggcattcctcttagcggtcaatttgttgttagaaattcatctgcttatattgatatatctgttcttagagatgcaatagccgatgaagacaaaaatctttcattggtgctcgctaatggaacaagcgttactgcttctataatgctcaaagatacttctattgaacctttatttgaattgtattttgcgaaggacgctgcaggtacacaacctataacaaaattggatagtgggtttcagttttttgttatcgttaagggcgttgaatatgatccaagcgtagaataccatattgattttgaaggatctactttttctgaatatgttttaggtggtcttcctagtcagattgctgttaagtttaataataacaatcttttttcatatccaataaacgtaaaccaacctagtttagatgaagaggttgtgaacatcacaacagaccagtctaacctgaatctttacaatttagctaagactacgttgggaaaaattagcgacccttgtaatatcagatttgtcatttcttctggcgttactgtttcagcatctaccgttttacaacctgcgataacagtaggagattttccgtacggttctaccataacactagtgaacaacggcaatatctacggtagaggaggtcggggtggatacgctccttcatcaatgtctattaatcagcttgacggtgagaacggcggtgcttgtattgacggaagtcctactgtattcttaacagtgtataactacggaaacgtaatgtctggtggcggagggggtggcgcttctggcgtttgttttataaatctaaatggttatagcgcaactaattataatccaattatgtctggaggaggtggcgctccttatggcgagaccccgccaataatgggtgcgggcgtagaattcattggagcaggtaaaaactcccaatcgttgatagaatataccccttcatacgctttgatacgtcaatggtattctaaggataatgttctgtacaagatggaaggaaatgtcacaactgtaaataataaacaccctgtgggcgacgcaactgcaactctaggcgcaaagtacaaagatataattgctattccatcttctgccggcaatggcggagatttaggaaaagatggcacagacggcattatggacaacgttaacaccttttattctgtcgtcgttaatgcgggtaaaggcggtaaagcaggaagaatcattctccaaaatgaattcaacgtagatatcaagaacatatctgggatagtgagtggtcattaatgagcgcattagcacaagtcaatatcaaggttggtagcaacaagctaaaaacagctagtatggtaattgaacgcgtgacgcgttcagctaccgtttctccagtagtgagagatggcgttaaccaactcgtcactagcgtagaaagaatgaagcagcataaagtatccttttcatcattcagtggattggtctcaggagacgaaatagttgttattcttcaaacttctggcgacaatagttcaggaagtaacttcatcgaattaccagctatcagattcataaacggaacagagtgcgtatggactttttctataccttctgattggaacggttcattaattgtttcagctcttcacaaaaactacggtatgaaacaaattggttaccgttttttcaatttgaaataagtaaccaaaagaggaacatcgcatgtcagacaacagcctatccaccagatttccggaaggcatccaaacgcctaacatctctggactaaaatatctttcacttgaaaagaatttcacgctagataccacttcggctgctttgaaagtgttaagaaagtcaaatgaatataattttgttgctgatgagggtaacgtcatcaacggcatttatatcgttgacatttctctaaacaaaaagcgtttcaatattgtaatagctgcgagtggtcagctatcatcagcagacttatcttcagatagcatcgataacagtattaacattctgagctcaatgaatgaagacccttctatttttaaattcaagggcgcgtacatagcaaaacgcaatgggaatcggaacgtcggattgattatctcaatgacagcgatatctggtttgtattctgaagctctagtagaaatcacatctatgagcgttcaggggcgtttgaacccttctgtagattatcctcaattcacagaattgaggaatcagagaaaatacggctacgtgaatgctattgaggtgaaagattcatctaacggcgtgaacatagaccttaatgaagtggtttattcgtctgtcgcttcttgtaatccagctacagctggttctagaccgaatacagacggtattggtatttgttatacaatatctggtacaaataatacatccatcggcgctcaattgtttatcgacctccagaatgatatgtattatcgttctagaaacaatggtgtttacggcttctggactcaagtgtttacgtccaagcgctatccaagagctgtagcaggtggatctgatattttgagccttaatggttccactccagtaaccatttcagtacccaatatagacttgtctcagagtcgttatatcattaacgcttctccagagaaaattctaactgactgtcaaggcttccagattgttagtcgtacaaatggcggttttggcgttgtcgctaagtctcctaccgctaacacttggaatggtaaggtcaattgggttgtgtatgaatcgaagccttcggaatctgatgtagttctaggaatcagaatgggcattctccaaggttcattattcacagaatctactcagatcagcgaaggtcaaaccttacgcgtaattgtgtctgcaaatggtcttgcgaacggtgaatccttagcatataattttacacaaatatctcagtctgacgtagatggttctttatcaggatacgttactattcagaataatcaaggtattgttgaccttccaattgtacaagacttccttccagaatctaccgaaacattgcgtttcactctagctgccactaatacatttaaagaaattagtgttctagatgtatctggaccaccagaatacaagagtcgggtgtctagattctctgacggttctacaactattactcaggctaatgaaggcgacactgtttaccttatgcttgagtcaaagtatgttgcaaacggcacatcttttaccttctctagttcatctgattcagacattgaaaacattgttgaatttggggctgcaagaaacattaacaataacttaattgttgtaccagtaaaatttgaagctgatgcaactaaaccgtacgaaggcactgagacaattacgcttaatgtacttctagcaggcgttgtggttacttcaacaaatattcaaatagcggacacagcttggaaggctgagtgttggtattctacagattctaacggttctacagccattacacaagctaatgaaggcgacactgtttacttcgtgctgagagtaccgtctggtacagctcaaggtacgttcttcaaacctaacattaagacagtcggcacaacgggcaatattgtcgagggcgatgatgtagacattaccaatagcaatattgtagctcagtcattcaacggtaatatcatattatctcaaaaaattgtaatacgcgcagaccgtaaaacagaaggcgatgaaattctatatatggagtgggtgaatcaatcaacctcgtctatagttgctgactctaataaaccgttgttgattaaagatacgtcaacaaccccgatccaaaacttgtacatttcgggcgaagtaaaacaagacgttgatttgaaaactttgtttgaaactcagtttggtactcaagacgttccttggatttgtaacctctatgttacaggagaatcttggtttgaggcttctgctacaaacaaattcgctgtaacttctgataattggttggctggaacagttgttaatctttattggaatagcactggcgggataataggaaagggtggacagggcgctgatcctaaagttcctagagttggggcagctggaggaaatgttttcaatattgggtctggtcacacattgaacatcaatctttattctggaaacgtaatcgtagcgtcaggtggcggtggaggtggcggtgttactatcaatggaaaatacgttggtggcggtggcggtgcagctacaggtttaggtggtactgctgactacgcaggttctcaagcatcaagacttttgggcgctcaaggtggaacgtacacccacgttgacgaagtaacctctgtatccacggattacattggcggaaagggtggtgatctaggaatggctggttctacttctacctctactggcgttaatacggctgcaggagctgcgggtttaataaaatctggtggagggactctagtattgaaaaaagatggcgtaacgattacatcatcgcagtattacaatgttaacagtttcatacgaggcaactggtaagaaaaaggggcgcaagcccctttcttatgtctaatgctaaatatagacagacatatacgttccaaaatatcggagattggccttggacacttcacacgctccattttatgatgattacaacccatctaaagggtttcataaaattctattcagacctcgagttgtacaagcccgagagttgaaccaattgcaatccattcttcaagaacaaatcaatcgaatgggttctcactttttcaaagatggctcagtcgtaggtgagggatcaatctcattaaaagaacaccagaatatggttaaaatagttcttccagtgaattttgatgcgtcctttttcgaaggcgatgtttctttgaacgcaagagcatctaatgctggtttgaatgctaagattctgaagtatcttcctatagatggaacgtctggatggttagcaattgaataccttaactcagacggcgctgaaggcgctgcatttgaagatgcatctacaatcatcatctctcaagacatcgatggttcctctgtagacgtattaatgcctaccgttgttgaaagcgctttaggcttctacgcttctgttctagaaggcgtttatttcgtaggcggaaaccttgtctggacgaatagccaagatattctaattccagaggaagagccatctatacttgttggtttccgtattgatgaagaagtgatcgacgaaactgtagacccatctttatattcaaatgcttcgggtgagccaaacttcaaaggcgctggcgcttgccgtctcaaagttacattaaaacttgaagcgatagataaatctgaaaaagatgacactttcattgagattgtagacattgacggcggtgtaatccaaagtgtaaatgataatacgcaatacgctgttttcaatgatattctagctcagcgtacctacgaagaatctggtaactacactgttacacaccctcctattgaattattggaacatctacgcgatagtgaccacgtagatggtctatatacaaacgtagaaggtggtaaggaagacacgtttgtagcgcgtctgaagcgcggtgtttcttacgttcaaggataccgtcacgaaattgtgggttacaaagacgtagaggttggtaaggcgcgagatacaaagattgttaacaatggcgttcttcaggctttatacggatcattcattgtagttacaattgataaagggcttccgaaacttgatctaacgcgccttcacagcctagtagatgaaagcaattcgctcttgggtacttgtaaagtgcgttctatcaaacgcgaaggcaacaatgcgatcctttacttgatggatatcaatatgtctctgggtcaatcatttggctctgtgaaacgtattttaggcagcttctcgacttctgatacctttgaagctactttggttgcttcaagtgttttcggtgggacaactgatagccttttattctcattaccgtactcaaatgtgagctcattgacaggcgctgctggtcaagacgtgtcattcaatgtgacgcgttcatttgacgtcacattagatgcttcaggtaaggcttctattggtcttacaggttctgaactattcaactcagttaacagcttcgactattttgcgagcgtaaatggcgacacggcgcttcctatttcaggtatctcactagcaggttctccaaatggtaaggctcttaatctagactttggtgcatcttactctggttcagttgttcgcattgttgcttctatcgttaagaccaatccaacttacaaaacaaagacgttgagagaagtatcagaaaacatctctttcaatgaatcatcttctgttaagttgaacaaagctgatatttttagaatagtgagcgttaccaatactgatagcggtttggatatgactcctggtttccgcttctttgatggtcaaagacctaactggtatgaagtaggtgaactgatcagcactagtggtaatatcactgcaaacgtatctgtgacttacgaatactttgaacattctaacacaggcgatttcttctgtgtagattcttatgatattgcgcgtaccgatatccctacatctaaagaagcgtcagaccgcgttatccgtagtctagcagattgcatcgacttccgtgttctgaaagcaagtgacggtagcttcacgaatcttccagctgtatttgatgtaatcaacccgaatgacaacgtgcgtattgatttgacaatgtatctacctcgaattgatgtattgtacgtttcatcttctggaaattactcagtttctcggggtgtaccatcagagaatcctaaagcgcctactataccagcagacggtatggctttgtatcaacttcacattccagcttacactgactcagtgactcagattaaggcaatcaaggttgaaaacaaacgcttcactatggctgatattggtcgtttggaaaaacgtattgagaatacagagtattatatctctttgagtcaaagtgagactaaagcagagcgtacacagattcttgaccctgttacaggaaataaccgcttcaagaatggtattgcagctgatcgcttcaaagatttcaaactggttgacgttttggattctgaatgggctgcatctattgataccgatctagggcgtattgcacctcctattgtacaaaatggcattgacatgacatatctttctggcgctcagaagcatacgtcaactgtatctcttccttatacattagttgaattcgctagtcagccgtatgcaacaacaacgtctaacatcaacccatttgcggtattctcatgggcaggtagcgttacgcttacaccagctcaggatttctggcgcgacactgtttacaataaacctattgtgttaaatgtgacagatgatctaaccaatggactccaagctggtgatcaacttataggtactacagtttcatcttcattccaatggtctagatggtggagaaacacagttgaaacgactactcagacgtttctgaatactaaaatcacatcctcaactgacgtgaaggttgacgattctcgagtgtctttgagtattattccttatatgcgtagcattgaggttaaattcgatttacgcggatttaagccactaactcgattgtatcctttcttctcaggcgtagctgtttcatcgcaatgcgctcaaacaggtaaagcctacggtgaagctattgtcactgacgtatctggtaacgtatcaggcgtattcaaggtaccaaacactaatgacttccgtttcaaaacaggttctaacattctgaaattcactgactcagtgaataatgacgacaacccgaataacaactttacacgcggtgaagcaacccatctatcaggcggtgaattagaggtgcgtcaaaagactacaaccatcacaaagacactgcgtgctgaacagacaaaatggacttcatcaacttctcgtgtgacagcgcgttggaatagtgatcctgttgctcaaacgttcaatatctctaaagacggtggcgggtttatcaaagcagttgatattttctttgctacaaaggctaagtcaattcctgttacgcttcaggttcgtaacgtgtacaacggcgtgccatctaatgaagtattgacttttggcgaagttgttctcaatccgtctcaggtgaacatttcagaaaatggtacagttgctactcagtttgtgtttagcgaccctgtgtatcttgagaaagacagagaatatgcaattgtattactagctgatacccaagaatataatgtttatcttgcgcgcatgggtgagattgatttatctaaccgctatgttgttgctaaacaacctcatactggtgttttctttgtgtcatctaacggtcaaacatggacagcaaaccaactggaagacttgaaattcaagatttacggttgtaagttcaaaactgattctgcgtacgttgcaacattagaaggcgcttcgcctgaagataaagcattaccatacaactcaatttctacaactttggactctaatattatcacctttggttgcttaggtcacggcttaaaagtaggcgattatgttgaagtatctggatctgtatcaggtaatggtatagacgctaatctaatcaatgtgcgccaccgcgttacagcagcaaatgcttcaacattctctgtagaaacaacggctgttgctcaactaacaggggctatcggaggtggttctattgctatcaaagcgaacaacccatacaataaggtttgcccgtggttagggtcattcattctggatggtacgtctttgaacattgatctgttaactcatcttcaagaaaatagaacggcggttacattacaaggtctagagagtgatacatttgctgatatgccttttgacgccgtttcttcatctaccttgaagcccaagttgaatctaacattgaagtctgataatgaatggttaacaccactcgtagacctagatggcgcaggtctagaaatggttggtttttgtcttaacaacgatggaaacaatgttcttgcaaacgccgttacgcgttctttgaagttcaacaacccatccacttcagctaaaatctatgttgtgggacgcgtaccattcggttccgccgttaagttgaaatacaaagctatcacaagtggcgatgataacttaacactgaaagaatgggcagacgtagaagtctcaaaagcgttcaagaactcttctacagatatgtacgaagctgagtttgaaataaactctgatgaatctttcctaggtctgaagctcaaaattgaaatgctaggtgaggttggttctcaatctccagtattggaagagctgagattgatcgctttggcataaaggaagacaatgaataaatggattagaaatggagcagctgtgtcaaatactgacacagctggctttttacaatacaccaaacaaagagaattagcaaaacagaaggacgaccaaattcaggaactccagactaatgttaaaacgctaaatgaacgtttggatagacaagaattgttgttacaggagattctgagacatgtacagaaatagaacacgcaaagatgtgaaggcgcgtatcttaaacagtttaggtcatcctattgttaaggtaaacataagtgacgaacaacttaacggagccattgacggcgctatcaagaaaatgtggcgctggcacaataacttttcatttgaatcattctgtgtagtgaagatcacaaaggaaattctagaacaagggtggttccaagtaccagattcttttgatgcagtaacagacatcattccagttggaaatagcaatacagatcctttgtattttgcgaccacagaatttacattgacgcgcaatgtattcttaaacggcgtgtttatgacacctctgtctatggttgattactttgcggcaatggctcgggtagatacctataagattgtcacaggcatggacattttcccgtttgtattcacaaagtttaatcatcaaattactctgaagtttcctgttaaagaaaatgatctgatctgtattagagcacttgaagactatattcctgaagaggaacatactggaatgtcttctgttcccacaagtacaattggcggaggtctaatctttgataatgaaacaatgcaagacttagcaaccgcattgtgcaaacaaatcattggtcaaactatgtccaagtatgaaggtatgcaacttcctggaggacaagttattcaaggacaagctatgttggaacaaggtcgccaagatgaagctgacatcatggagcgtttgagaaatgaagcttgcgacctgttctatttggcttaaatagaataaaggagacactatatgctaaatccacacttaaacgcaatacaggtcacaaatgctgaatgcgaactatttgaccagatggttattgaagcaattcaggtcattggtgtggatttagtctatatccagttacttgaaagtaatcataatgctttgaatgacgctgacgttaaaggtctaaagcaacagttcactattgaagctcaaatcatggagattcagagctttggcggcgagtctgatatattcggacagtttgggttcactccagctgattctgctgtctttaagatggctataactcgttttaaggctgaagctgataagctaggaatcactgaaccacaagagggtgacgtattatatgacccagtgggtaatagactttgggaaattagacgcgtacgcactgatgacctatactacgttggaaacaaaagatattgttggattctaaactgcgtaatctatcaaccaaagcacgaagataatatcgatgacttcagtggagtcgtgttccaaggtacactaggtgatcttgttcaaccttctcaagatgagtcagataattttgtgatggagtctgagaagcacgtaactgatgtaccagactttgacgtaaacaacccatttggagaataacatgagcatctccctattctcagatggctctcatagagtcgacacgagtcagttgtcaaagtatacagctgtcttcggttctttattttctggtttcggtataaagattaacaataagctgatcagcgtgcctattagatttggtaatggtaatagcagaaataaagctgacactcagttaaatgtagatgagcaactacaaccaaaagtgaaaccagcccttccagctatggcgtttactctaggctctattgttaaggatggtcaacgccaaactgtagaccacgccactatgcgtagtcgcaacctgtacttcaacaattctactgctaacagcaagtctgcgatgtctccgtctccgtataacgttgattttcatcttacagtcagagccaacaatacaacagaagcgttattgatctttgaacaaattgaatcagcctttgctaagggcgtgaatgtgagattgatggattcagaccttctagatgtagaacgtgtgatcaacatcagaaagcaacctaatctagacattcaagacaacttcgagaacgttgatgaaacgcgtattgttgaatttgaacttgtattcacactgaagggctatctgtataatcggatcaatgacgtacctgttattcttgaatttgatgcgtctatattctctaaagaaaatgcttgggttacagatcatgtaaatacagatgaattggaaatacgctcgttgaaacaagcgcttgcggagaacaaagatgaatgatattttaagagaattgggaattatcccagctggcgctgatactaatacagatgacataaacgcagacaatagaccaactcgcgaagaatttgaaatggaacagaaggcatctaatgttccagaaattgaagactcagacgccaaggttgactatgaatacgctcgtagtgctatctatgctatcgtgcaaatgaacctttctatcacacacagagcgatgacagtagcaaaagagacagaacaccctagagccattgaggcgttcaactcagcgtcatctaacactttgaactcggtcaaggaattggtcaacattcagacttccgctaagaccatttacaaagatgctaaggtcaaaccaaaagaagtacctgagtcagaaggtggcacaaccgttaacatcgctcaaactgtagccgtgaacacttcagacctactgaaacaggttgatgaagatattgcgcgtgaacaggctgaagctgagggtaagctgaatgaaggctgatggaatcaatacgttccaaaagagtggcggtaaacacccttggccagttgacgattgtatcttatcaaaagatcaattgtttgttggtaagccgagggtgaaacgcgctggtgtcactattgatatgacaccagatcagctcaaagaatggatcagatgtaaaaaagacccaatctacttcttgaccacctacggtcgagtagtgcacattgaccacggtcttgtaccattgaaactctacccttatcaggaagagatgatcaagaactacaaagaacatcgtttcaatattgctttaacttgtcgtcaggcaggtaagacaacaggtgtagtaggcttcttggcttggtatctgatattccacgctgagaaggactgtcacgttcttgcgaacaaggaagctcaggctatggagatcatgaaacgtctccgccgtctgtatgaagaattaccatacttccttcagcaaggtattgtagcattcaaccaagggaatatcattcttgaaaacggttcaagctgcgaaggtcatgcgagttcttctgactctatccgtggtcgttctgcttcattactttatcttgacgagtttgctttcttgggcttctcagacggcggtcaagactttgttgactctgtatttccagtagtatcttcaggtaagacatctaagattctgatcacttctactcctaaaggggcaagaggtcagttctacaaattctggcaaggagcagttgctaagggtgatgattggaatggttacgttccattgagtgttacttggaaggacgtcccaggacgagatgaagattgggctgtaaaaactaagaaacagcttggttcttctcgattctcacaagagcatgaatgtaaattcttgtcttcatctggtggtttgatctcatcagaggttctagaaaagctcaagattgctacgcctgaatatacgtttgaagatggtgcgtacgagattctctacccacctgaaaaggatcgcatctatgttgcaaccgtagactgcgctgagggtataggtgaagacaactccactatagccatctttgatgttacatcaaagccgtacagagtggtagcaacctatcattccaatactgtttcacctctagtgtttccgtatgaaattgagcgcatttgttctcagtataatgaggcttgggtgttagtggagaacaacgctatcggatctcagatcattcagattctctactatgagattgaatacgagaacgttttcctgacttctaacaagaaaaatggtttgggcactcagataggtggagcaggggctaaaccaggaattaagaccactaaatcgacgaaatctgttggttgttctaacctgaaaacgcttctggaactcgatattctgcttattgaatcagaaatcatggttgatgaactgggtacgtttgttcttcagacaaacggttcttatgcagctgataaaggcgctaaagatgacacagtaatgaacttggttttattctcttggttgactaagactgagttcttcaagtctgaatatgagaccaaagagggcgacagtattcaagacagaatgaagtccctcagagaagcatcaattgaagatacctctatgacttcattctttagcaccaagtctttggaagctgaagaggaaattgaatctcagcgtcaagaatctctttatgacttcatgaatagagatgatggcccagcttatggttcagacggttatggaggttatggcttcaacggttggagttaaacctaccgtccagcctaccgtgtggcgttggatttttacatacaaaagcccttgtctagtacaggggctttttattggattttacgtttctttgttcatgtaacatccttgtatttcaaaactttagtcttgttaacaaaggtgcatgaacttgggaaatggttaactgttttgaaaacgttacctgcgccgtcataataagttgaggtttgatattccaactcatagataacaccgtattctatatttctccaattactgcagtgttttgtggtactgagacgttggttgatcgatccatctagcgctttaacgtgaacgctgaaatgtttaggtctgtttatatcaatgacttctacgctcgtagtcttgaattcagttgttaccttactagggtgttcagatttagagcacccgagtaatacgagacaaactacagctaggatgaatttattcactatcaaatactccataccaatgcatatcctcttcctcgcgtctcgctaattcatcagctgcgcggttggcatcagattgatttttacagacagataagatgtcaagcgttttacccttgacaatcttgttggggttacattgccaacctctagattttcctaactcagtttggcaactgatacatccagtacatttagattgataccaattgtagttccaatagatacaaacgcaccattgcttatccaaacggcgcacttgaatatggttatatccagaaggtttatgtttgcgtttagccatttttattttcagccttgaaaaagataaatgcgctaaattcattggactctagcgtgacgttcactatagaagcatcttccatatccataagagctttgtacaggtctttgttatgagacaaaatcttcagcgctctaaacatttctggatagacgtccatttttggtgtcatgcgttctgggtagtgtttaccgtaagcgttgaatgtaatatcccgaaaattatccatgtggttatagataaagttcatcccaacttgtccaaccgtttcagataaagttgagaaaggaagtgagacagaagccatagtcatttaccatccaaataaataaaattataaagtattattgcgaaagaaataaaaatcaagaagcccccgaaggggcttgaaactaagcggtggtaatgatgaagagaccattgtagtcccaatgtaatgctagacaacggcgtccgttgacctcgccaacatagaggtcgcctagaatgaaaccgtcattgcgaacatcgcgttgaataagaaccatatctggaacgtcagaagcctcaatggcgccactgttagatgggcggacaactagagcctcagaatcgcccacaatagatttgtaatgaggagctacaagctcattggtgagaattcgttggatgaaatcttcgtcaattagaccagaaacgtagtcaaaatctaaatcaaaatctactaagcctaaatgtgtcatgtcttgtactccgttgagtgtaattaaataatgaacttgttgtccatgattaaaattatgtgggcgtaccagagaaaagtaaaggaaaaaagaccgaaatattcaaaatatttcggtcttcaattattatgcaataacttctttcatcagcttgaagccttcttcagtgaatcgcactgagttatcttcatcaaagttatcaagtacgtcaatgataccctttttagccaatgatcccaaagtaccctttacagtacgaacagtcaacttagcgtattctgcgaatgtatcagtgtaacaccaagtagaaacgtcatcagctgattctgcaacttcagggtgtccattcacttcacaccattctgagtaagcaattgctttgataactgcttcttcatttgcagtgattttgattgagaattcttttgaagatttagttgatttgcgtttagctttgggcttgtcttcaactacttcttcagttttgatcaagtgagcaacagcctcaagacctttaacagtgtattggtattgtgaaccagttttctcttttttagaaacaagaagttctttgtgaatcatagagcaaactaagccagatgcaaccaatttagttaaaccagagatgtctgcgatttgagtagttgtaacccaaggttgagtttcaatagtagtgtttgtagctagagcaacaagtacttgagtttggttagcagttaatgtagtcatttttgatttactccgttgagtgttattgaaccttatgagattcattataccctaaccatcaacggaagtaaaggattatttcggaaataatcaaatatttttatttccagtacttttgaagcatagcagttctatggtgcgcgcccatagaaaataaggaatcttcaaattctttattttctttgttgcgtattcttttttgacgcgaagacctcgattcttgatatttgatgctggaagttttagatataaaagccccagagacggttctagaaaaatcttcgagtaatttgaattcaaacatctttatctcctaaaatgaaaacttaggggcaatagcccctaaattctctcaattattagttttgattttgtttttcttatttttatagatacattctgaggtattttaaattgatcatagcttttatcttcaaacaaaaaagtttgtctaatgactattagattgctatattgttcctctatacaattcagaccaaattctaacaatctttgaagagactgttcccaatcttcgccgttttgtataataagaatatcttcgtgttctaactctgacatactgtattcaacgcctaatggccacggcgttctaaaatcttgtctcagagcttcgtaacgcagtttgatgtaggctttattcttagaaccttcaacatccttattaaccacttcatttgctccaaagtagacgcggaacaccttaatgttatatcgaccctttacgctgtcgcaagggtattttgtgtaaagattaggcatgaagaagaaaaataaccagtagtgaattggcatgtaacgaagatatgtaaacagaaattttatgtatttcatctattaatctccgtgtaacgacaattagaacggtaatgagtgacttcagttccatcatccatctttagatacaaaaaggttttacctccagatattctgttttgagatacatttttgtaatgaatatcatttgcgccaggaactatacagacaatgtcgtagtttcctacgtaacgttgttcctttgaaggtttataacaaccagctagagcgattgtaagaaataatatgaaaaatttcatgaggttctcctttattggagattattatagtgtttttattttataattgtaaaaaatcaatttggcccattgtctagggaattaaaccctagtctttcaatacgtctgtttaccgtattgacgtgcaccgtacactaacgctggataatggtgtccctgacaaggattcgaacctgctttctctccgttatgagcggaacgcttataccatttaagcttcaggaacatgttaaaggagcgtattgctacgcccacggcttttaccgactgccgcgttgtccatgctaataacgcaaccaccgccgtgttctgggtgatactgttgtcctcgcatactggagtttgaaagtggcaccccgcataggattcgaacccatgtatagcacactagaattatgctgcctaatccactcggccagcggggcgtagtcaaactacaacgtaataacaactattgggaataacccactccatgacccattcacttagtgggcaacaattgttattacgttatagtctgtccattctcctcttggggttgaggaaggaggacaagccctttgtcaaatgtagctacccaagttcaagttaggctgagccttgacatgaacagtattatatagtcaagacaaattaacggcttaaaatcaatttatctttcgttacccgaacagcaaccttatgattctttggtctataaccgccgtcaacgttacaataagatattgtaatattagacgcgctaagaatcttgaacccgataaattcattagccatttagttgaccatccactttcacgccattgacatccgcgtctacgtcatttggatcgccttgaacgttcacttcagtacggcgcgagttgtcatttacaatagagaagccattgaaatgacagtgttcaattgtggctgacgctttgaacgcgtctagggctttcagagtgactttatcattggtgtgatttttcgcagctgtatccagaatacgaacgatcgcatcagttgaagccttaattgatgcctcatctacctttacagctacgcttacaaggttggtttcgtttacatttttaacgtcagacattttgttctccaatgatgtcttaataggatttgaggtttagactttgccgccgataagaaatattttgctgtcgtctagcggattgtacgctcgaccattagatgcgtacggaaatagaacttcaacttggtcagtcttagcaggtccttcaatgaacagtaccaccgcgcctttactgaagcgaatagtgtggatgtcttctgaggtcatttgataccattcgccttcactatgaagtttttcagaaatctgaaacgcttggcgttgttgtctgttgaacttatagtctcccaactggccagtttgaatagtcacgcaatactcagcgtgaagctcattattttctttgtatttgctattttcaataggcgtatacaagcgattcaatacagaacctttgagcaccaaacacgcaaaatcatatttgtgagagtgcggtagacgaacttgagtttcgtagttagccgtaaacactcgaaacataccttcactcaatagagttgagtctaagccgtgttctacatgatttacaatacgcgtggactcgtctgccattgaaaggataaggtcacgcaaaatttcgagattcatcgtttggtctccgttgtagttcagagaattatactatatattacggtttgggataaaaaatcaaagagaattgatatgatcgacaataatattgaagttggacgcattgcggttatgatatcgcttcaagctgataaatcttttggaaacgttggacgcatcgcatctttcaacggcatgagagtagaggtggaacaaaatggagacgttatttctacattcccttcaggtattgctctagtttgcgatacaatggaagaggtagaacgcgctatgacttgcgatgaccaaacctacgagaagatcagagaattgaagaaggctagacaagccttcattcgcaacctgcaatcggaaagtagtgtttctcaagcgtagacattatatgcttttgaaacataatgagggaagtgactgtttcaaatacgtctgcttccctttttaacgaaactccaatcattggtaccactaccccttgtatacggttacgaaccatgctattaccaaggaaaaggtcggtatttattggtagaacttcagctgaatctgttaaagctagttcatatttcaagaataggtcatgttgaagagatacagctaaagtgcttctattcaaaacatcattcaagaatggagtatgagctggtagatacaccgtgattctttctctttcactcttcacgtaggctcttttgtaataatctaaaaatgatagatcatctatcaactttttagttagctctattctctcgctatcaaaagattgatctgattttatacgttcttgctcattcatcatcttactcctagacagaatgctattaaagaagctactaaagaagcagccaaggataaaggcacaaacacgctagggtgaatgagtttgtcatttgcgtaaccagctactataacaaatgttacgcagtacaccaagcatatcattggcataatgtcttccattaagttatcataaaggtattaatgtaaaaagaaaaaggagctataaagctccttttttccaaagactgtattttagaagcgcgagatccgctgatctaaaatgtcacggtaccacaccatggcttcaagttgttcagtgagacttttctgctcaatctgagacaaggtaaagaatacacttgtaccaatgaatgactcaagaccttcagcgcgttcattcaactgagcacgttcaatatccactcgttcttgatgaggccacgcacgctcaggtaattctttaaaggttttttcaaatacatctttaggagaccaagatacgtgaccatcataaccgtctaaattggatggttgaccactttgaacaaccataaacccctgatctgaaccatcttcatcttctggaagagtccatccgcgcaatgtgttatagtctagacgactcattgatgcagccgtaattatctttgtaccgtaatactttttcatgatcatttacccacgttgatgaatggaagagcgccgttgccaccgactacagtttcaggaagtttaccagaccacttttcaatagctttcaactgtacatattcagtaccaccctgagctcgaacagcttcagattgaatgcgaatagcttcggcttcagcctttgccatttcaatcttttgtttcgcttctacttcaatacgcttgaggtcattttcagccttcaatgcactttgttcagcaatctgcttagattcgattgagtcatagaaaactttggagaaagtgaattcagtgacctgaacatcttcaactacaataccgtacttggcaagacgcgccgttaaatctacagacaaggtatttttaacatcatcgcgttgttttgtcaattgatcagccgtgtatttggtcacaactgatttagtagcttcttggatacgcggatcaatgatcttcatttcaatatccaaaccagtctgactgaacagcttttctaggctagacggattaatatgatagttcacaacaaccttggttgctacgcgctgcatatcacgcgtattcgcctgaatagccgtttctgctttagctgttcgagtatcaacaagaatcaccttttgcataaacggcattttgaaatttaaaccagagtctttaatggtatgcaattcattgaattgagttacaatacctttatgacctgttggtacagtgaagaacagagatgataagaagatcaaccccacaataatagcaataacaatagcagcaattttaggtaaaataccatactggtttaaatttaagcgcattgcgcctttctcccatgttgagtgtaacgaccctgtctgttagttgttagggcattcacaaattgtttgagctgtttacgctctacaaataagctattgaagtcatcaaacttcactagcgccaatctgaagtctagaatattgatatacatgacatcaacatcaccttcataagaaactatttcatcgctgaaatttgttaaaacataacatcaaatggtaaaagaccgtttttagaccatatagctgatgtcaactctacaacatcatcagattcccagatgtctagatttgctacacaatctaataaaggactcacaccttcaacatctgagggctttatctcggttgtgagaaagatgtttttaccatcatgttccatttttacactaccgacataaaacttcatcactgcccccgtgatactactgggttgttgccgaaccctttgattgtgtcttttaagtgccccaaaacatctttgtttcgtagcgcgttgaatctagacttttcagcttgtttcaataattcaacaatcttcaaaagatcagattcagaacatttaaacttcagatgcgctttcaatttttctaaaggcataccatcagtaataatttcagaaataccttcttcaaaaaagtctaactgaggtattttagttagattgctaaggtctaaatgcacgatgtatccagcttcaatgcgctgttgtatttcgtgtttcagattagaagcgccgtagcagccatctattcgttgttttaagtcaatataaaagattgtcatatccatttcaccaaagtgggggatttctcccccgaaaacaatattatatactaaaaagattcactgaagaagaatcaatatttgttgacttttcgctcttagaaggaatgaagcgccctttcttccatccatctttcaaatggttatccaactcatcggagtatatcattttattgatctctccatttctaacccaaattctgctttgaagctttttaaccatattagctttaccgtcttccgacatttgtaagtgtctacctttagaccaccctttctccaaatagtcgtctacttcgtttggtaaaactgtaattgatcttccatctgaatgttttatccatactctgcttatctggttacgttcaagtagaatctttttatgacggtctgaacctacatactcacttttgtatttttccagttcccctgtttcgaatctagggtcatctacaacagcttgaatgattttacctgtggcaacgtcaacagcgttcattttaccagcatttacgtgtttatgaatcttagagtcatatacgtctgtactgatcttcattctacgaccattaggaagaatgatattggttttaccgttattgataaaagtatgtttggaaggatcataatcatttaagttgaccatcatgaaatttccattctcgtcataaacggacttcaaaccttgttttattatagtttctttaggaggaataacaacgtatctaccaactgttgacactggattcatgatctggtaactgaataactcacctgtcttaattctagggtcgtcaacatcaacccaatgtttctctccgaagtgatctagacataggactttacctttcatacatttagacatatattctgaatagataggtctaatctgttcttgaagtatttgatattgtctacctgttaatcttttattgccgtgagtagaactatcattattcattaaccacatagcttgttgcatttccttccactctgtacctctccataagagcaggtgagcaatgaaatgttctctaggcgttaatgttactagattccacttttcagttctgtattctggaaatacagatttaggtagaacgtgatgaacatgattgctttctttatctagaacttcagtttgtctcttttggacaagtaatagataatgcttcatgtatttgtttcgaggtagataattttggaactgattcatttcattcctccacaggattatgactatttgtatcttgacaatactatatagcacataatcctgcgggattgttactagacgttacgctttaaatttgtaatcgtctgtgtactgagtatgtttaccctgtttccattcgtctgttacgtctttaatgacagtgtaacgacatgtgcggcattttgaggctgcgtaatctgtgggtatcgcaacaatatcgcgtggatctactttcactaacactaggcggtcattagagctgctgaatgacttaatgtaggattcagagcaaatgtgtagaccattagaacaagtatttgtatcttggtcatccacttcgtttcgaggcatctgaactgttttgcctacgctgttgtcaaatgtaccagagtgtttatctttgtagtcgtggcgtactactttgaacgcaatgatatcaccatcttcggtaatctgtaagctgttctttgagatgaagtcaaagaagcgttggttgactttataagaaacacactgacgcaccttctcaatgaaagcagagaagtttttaaggcgtttcgtatcaccagaagcagccaagcgcagcataacttccttcagaccgccaacaagaggtttaccatcccaacgcactacaccgttgtcaatagaaagtttaccctgcgaccaagtcttcagctgagtaacttgttcaccttcaataaggttttcaagttctttggcttgttctgttgttaccttattttcttcaaccttactcaagaaaatattgatttggttgaaacgtgggtgatctttcttcacctcatgactttgagagccaattgcaattactgtgctgtttggaagcgtaatgacctttaactcaaccgatcctgaagttttctcttgcgttccaacttctgttttaacttctgtcgagcttgtgtgttcagcttcactttggttttttgctgaagccttcttgcgtcttgaggtgctagaagctggctgacgctttgctttcgctttacgagacgttttgacttcaacttctttaccgtctttatcagtagcttcagcaggggcatattgttcaatccagcttttgatagtacggttcgcaataccaaattctttagcagctgagttaatagaaccaccagatttgatgattttacatgctttgatagcataatctttcttttgtgcatctgtgtatttgacagccattgggcttctcctagtttggaactgacatattatagagtcagttccagtttatgttaaaaatcaattttggaaactaaaatcaaagatggttctttgtttctcaaaacgtattgcagctttcaacgcatccgattctgataaagtattcagagccgtataaactaaaaactcagtaaaggtttccggagcagaattgtttttcttagtcttttccactaatgattttgcgcgtttttcaacgtaccctccttgtatgtttctgaagatattgtttatcttgcgtcctatttgagatttaggataatgttttgcataatacaaaacagatattgcatccttgaagtgctgtaagtcttcagtatcatctttgaaaaactttttgcgaatgttagtgctgttatatcttataccgtgaatagcattaatgaaattttggaatcttaattcacaattgtaacggtattctttaggattgtaaacgtcaaatatttgtatagcaactttgtagaaataatcatccaaataatctaaattctcacgagctgtgctattcatagttaccgattgataacgcaatatgaacatccagatagatttacattctgaacttaaagttttgaaccacttatcagcgtctgtcatctctgaaatattagggatgcctgtctctgagatgatgtctccgtatttcttagctgctgttttggacattaacaaaattgatttcccaaagtatctagacgtggtcatgatagaaagggctttcatatccaccttttgtttatgttctggatcagagtatattgcgtttgtatcaacgtcaaaataaccgtaagcgaagaattgagattcatccaaattgtcatcttcataagccttgaagtgctcttgaagctcagacatcttaatttgagatggttttgtgtcaaagcgcgttttcttagtttctggtacaaacaaatcaagaacagctggcatgcgtctaaaaccagcagcattgtttactttatccgttgctgccttagtgtacttcagctcagataaacgcacaatattcacatttacaggtaaagattctgtaagaatgtattcaaactcagaaaaatcataacttttgtcagcgcaacgaacaacgtatatattacgattagatacagttgtagtaggttctgctaaccagagttctaatgtctttgcaattggtttagaatttgtgtcaactaatatcacagtcatgttatcaaaacgcgacgcttgtccagaagaccaaatttttatatgctcatattgatccaatacaacctttgtgctattatacgcaacagaagcctttgaaaaagagtattttgggttgtaattcttaacaaccttatctactttctgccccttctcgtctgttgtttgtacaacatgcgtttcttcaaaaaatggcgcggaaaaagtccagtggaagtcgccacgttctgtattacaaggggcaaaacgcattgtcacatttaccacttcacccttttcgttgatgataggcttctcgtacatagaccattctttgtaattttccatgccgtttgtatttctcacaaacggaagcatatatttcatctctgaagaaaagtttgcggctaattttgttgcctcaagaaagtttttgacttcagaaaagtttttcaagtattcttccgtcttgtctttagtaaacgcatcccctacactatgcaactccttcactagattttcaattgtgaagtcactatattcaacgcgttcgcggtctcctggtacttccacgctaccaatagggacactaatcacaacaatctcattatgaccgagcgtctgttgaatagctttgaataattgttcgcattcaggattcacagaatctggtttgaatggatacgtcactccacccatttccagagcaaaagcgccgaagaattcacgttgatcttcagctctgtacgctcgaacatttccgcgctctagagtcatcaaaggtctatcatacggccatgctgcgccgtcaatattaggtttaggctcaaacatgaacatctgagtcttcagagcatcttcatatttcttgcgttgaagatcgcgtacatgaatgcggaaagctacgccgtttggttcgtcagttggagacttatccaaaagcatcaattgcggtaaactacgacctgaacggaagaattggaatgaacacttcacgccatctttgatacattcaatattgtaagagtcagaaatagcaaatggagattttgctccaataccgtatgcaccaacggctttgtttgtattagtttttgatgaagaatataactgagacagatatttgatagcatcggcttcattcaaacctgtaccttgatctttaataacaaggtacggagaaatttcagacggaaggtagatttcaaaaggcacgtctgcttttccagcttctacgtgggaatctcgagcgttggaacaaatttctcgaataactgattcaatcttatgtacgtacaatttgtcaattgacaaagagaatccaatagctgagtttgcatccattgtcatctgaatagctgtaccgcctgatactgaaacggtttgatctgtgtcgtggagttgcattgtattcctcaaaagaatggtaatatatcaatctgcgtctattatagagtcatttactgaaatgaataaaaatcaaatagtcgcaactactgctgaaccgtaaggtaacagaccccaaagtgcattagctgatgggcgtatgctatctccagactttattacggctatatcattagccttcaatgtaccgccattgacactcaactttccatcgaaaacagtgactaaaacggcgttacattgttctgtcatattctgtatttcttcgaatggtatttctccgcgcagtacagctagagaaacgtaatcatatttgtcatctacagtatatccaggatgacggaatgaatctaaaaagtgtctagctggaatatgggataaggtgaagactccacgccccatatctgtcatacgacttttcataaaatctgatatctcagaagaaagactcaaagcgtagtattgcataatgttctccaaactaccgtgtggttgtgtaaaatatatgtgtttttgatccatactttacagaccaaaaaattacaaaattcgcaatttactgatcaatgttttcgactatctcagtgtatgttaaaagatattctgcgtaaccatggttacgcagccaaggagttatagaaacaaccttattgacacattcctttgttagaaactcattcagttgttctgtatcactaaaagccttgaccttggttacttctattaaaccatctatggtagacggaactgagaaatttgattttccgtcatcaaacgcagattccaaaacctctttgaacttttcagatagttttctataccctttgataaatctaattctttcatctggatcaggatatcgcacccgatgaaaatcttcctccatcttgtggattctaaaatccagagaagttattatgtgctttatttcttctacgcttaaatccattgttagtcactaccttttgacgttgagaccattcaagtttagctgctaaacctttgaatgtgttacttttgattatcatatagatttc

SEQ ID NO: 2 - DNA, 372 bp, Artificial Sequence
  lysin protein derived from the bacteriophage
atggctgattttgatcctaattttctcatacacccgcagactagagatttgaacctgttgcgcggtaaaaaggctgtattccaagccttcaagaccctagtattatctgggattgaagattttattatgcgcagcgatatccacggtggaaatatctatcagaccttgtttgataatgataacgctttgttgagagcgcagataacatctaagattgaggagattgctagaattgacgaaccaagaattgttattaaagcaatagacttcgatggttcaaaacatcaccatctcattatcaagatcaagtatctgtacgaaaatgagctggagatgtctgaagattctattaagattgtacgcacatcttaa

SEQ ID NO: 3 - protein, 123 aa, Artificial Sequence
  lysin protein derived from the bacteriophage
MADFDPNFLIHPQTRDLNLLRGKKAVFQAFKTLVLSGIEDFIMRSDIHGGNIYQTLFDNDNALLRAQITSKIEEIARIDEPRIVIKAIDFDGSKHHHLIIKIKYLYENELEMSEDSIKIVRTS

(19) United States
(12) Patent Application Publication
(10) Pub. No.: US 2022/0073567 A1
(43) Pub. Date: Mar. 10, 2022

(54) ACINETOBACTER BAUMANNII BACTERIOPHAGE MIKAB48 OR LYTIC PROTEIN DERIVED FROM THE BACTERIOPHAGE

(71) Applicant: MIKROLIZ BIYOTEKNOLOJI SAN. VE TIC. LTD. STI., Ankara (TR)

(72) Inventor: Medine COTAK, Ankara (TR)

(73) Assignee: MIKROLIZ BIYOTEKNOLOJI SAN. VE TIC. LTD. STI., Ankara (TR)

(21) Appl. No.: 17/417,123

(22) PCT Filed: Dec. 23, 2019
(86) PCT No.: PCT/TR2019/051177

§ 371 Date: Jun. 22, 2021

(30) Foreign Application Priority Data
Dec. 24, 2018 (TR) PCT/TR2018/050871

(51) Int. Cl. C07K 14/005; C12N 7/00

(52) U.S. Cl. CPC C07K 14/005; C12N 7/00; C12N 2795/10032; C12N 2795/10022; C12N 2795/10021

(57) ABSTRACT

A novel Acinetobacter baumannii bacteriophage and lytic protein derived from the bacteriophage are disclosed. The bacteriophage and lytic protein derived from the bacteriophage both have strong in vitro antibacterial effects on pan-drug resistant Acinetobacter baumannii clinical strains providing experimental basis for developing a preparation for preventing and treating infections caused by Acinetobacter baumannii containing the bacteriophage or lytic protein thereof.

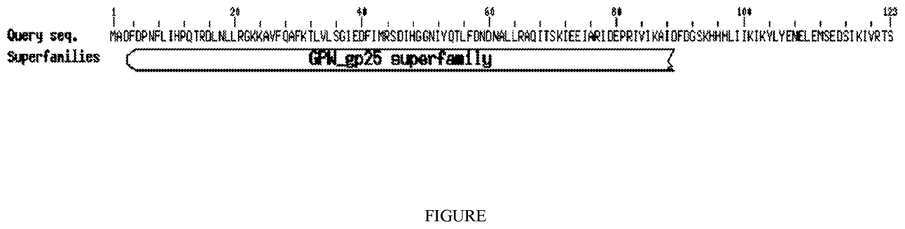

### CROSS REFERENCE TO THE RELATED APPLICATIONS

This application is the national phase entry of International Application No. PCT/TR2019/051177, filed on Dec. 23, 2019, which is based upon and claims priority to International Application No. PCT/TR2018/050871, filed on Dec. 24, 2018, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBUY138_SL.txt, created on 06/16/2021 and is 178,213 bytes in size.

### TECHNICAL FIELD

The present invention relates to a novel bacteriophage and a lytic protein derived from the bacteriophage specific for Acinetobacter baumannii.

### BACKGROUND

Acinetobacter baumannii is an opportunistic Gram-negative pathogen and one of the leading causes of nosocomial (hospital-acquired) infections. The highest prevalence of this pathogen is observed in immunocompromised and hospitalized patients in intensive care units. Common Acinetobacter baumannii infections include, ventilator-associated pneumonia, meningitis, sepsis, bacteremia, post-surgical endocarditis, soft tissue infections (particularly in burn patients), and urinary tract infections. Alternatives to standard antibiotics for treatment of severe Acinetobacter baumannii infections are urgently needed due to the emergence of multidrug and pandrug resistant Acinetobacter baumannii strains.

Bacteriophages (phages) are the bacterial viruses that can be found in all ecosystems such as oceans, up in air, sewage water, and in our body. The discovery of the phages was credited to two scientists; Frederick William Twort Felix in 1915 and d'Herelle in 1917 independently described the bacteriophages. After traditional antibiotics discovered, bacteriophage studies continued only in some Eastern European countries and in some countries in Soviet Union. Recently, however, due to the global antibiotic resistance health issue, bacteriophages have again regained interest worldwide and more and more bacteriophages are being identified and studied as antibacterials.

Many phages follow lytic cycle leading to the host cell lysis and death. Bacteriophages first bind to the host cell receptors with its tail proteins and then phage inserts its genome into host cell. Lytic phages take over the host cell machinery and multiply inside the cell. At the end of the lytic cycle, bacteriophage encoded enzymes called endolysins (lysins) hydrolyze the cell wall releasing the new phage particles.

Bacteriophages are specific for their host bacteria in species and even in strain level. The specificity of phages prevents harming the normal microflora of the human or animal body unlike broad host range antibiotics and bacteriophages do not affect the mammalian cells. Hence, bacteriophage therapy can be considered as a safe treatment method and reduces the side effects compared with other antibiotics. The application of phages has been studied as therapeutic agents to treat acute and chronic infections especially caused by the multidrug-resistant bacteria. Nowadays, the use of lytic bacteriophages and their enzymes to deal with antibiotic resistance crisis is getting renewed attraction by the researchers and by pharmaceutical companies.

Endolysins (lysins) are the enzymes responsible for the degradation of the peptidoglycan layer resulting in osmotic cell death at the end of lytic cycle of bacteriophages. Lysins are antibacterial enzymes having peptidoglycan hydrolase activity. They are part of the bacteriophage life cycle; however, they can be applied exogenously as recombinant proteins. Numerous studies have shown that, endolysins expressed in suitable hosts, purified and applied as antibacterial agent with rapid killing activity. Importantly, endolysins are so specific that they can target the bacteria at genus or even species level. This characteristic is important to protect normal microbiome of the human body. Additionally, bacterial strains either are slightly or develop no resistance to phage lysins. The scenario in the case of antibiotics different since antibiotics affect both the normal flora of the body and promote resistance in the host. Endolysin source is bacteriophages found in the nature so it is a plausible idea that there is a significant diversity of endolysin proteins. In general, lytic protein derived from bacteriophage exhibits wider spectrum of antibacterial activity than its mother bacteriophage. Taking all together the advantages of endolysins, they are now considered as potential antimicrobial agents to cope with multidrug resistant bacteria.

Bacteriophages are evolving with their host bacterium and they exhibit high specificity for the bacteria. Therefore, in order to treat the multidrug resistant Acinetobacter baumannii strains, it is necessary to collect many bacteriophages for improved antibacterial strength and to broaden spectrum of action. Phage and phage-encoded lytic enzymes are promising approaches to fight Acinetobacter baumannii infections.

Thus, the present invention discloses nucleotide sequence of the genome of the bacteriophage, vB_AbaA_MikAB48 (shortly called MikAB48 hereafter) and its derived endolysin protein sequence. Both the phage and its lytic enzyme showed high lytic activity against multidrug resistant Acinetobacter baumannii clinical strains in vitro studies.

### SUMMARY

A bacteriophage specifically infecting Acinetobacter baumanniii was isolated from sewage. Additionally, lytic protein of the bacteriophage was identified, purified and characterized. The purified phage and the phage lysin having strong antibacterial activity in vitro are well suited for applications such as preventing and treatment of of Acinetobacter baumannii infections and disinfection. The present invention particularly relates to the bacteriophage having a genome represented by the nucleotide sequence by SEQ ID NO: 1 and gene coding the lytic protein derived from the bacteriophage having nucleotide sequence represented by SEQ ID NO: 2 and amino acid sequence represented by SEQ ID NO: 3.

As explained herein before, bacteriophage MikAB48 and lytic protein derived from it of the present invention are capable of killing Acinetobacter baumannii specifically. The beneficial effect of the present invention is that the isolated bacteriophage MikAB48 has strong lytic activity and has a wide host spectrum on pan-drug resistant, meaning resistant to all standard antibiotics including colistin, Acinetobacter bauamnii strains. Bacteriophage or the lysin protein derived from the bacteriophage can be used alone or in combination with other antibiotics or antimicrobial peptides. They can be used in compositions of bactericidal agents, disinfectants, or therapeutical drugs as active ingredients.

### BRIEF DESCRIPTION OF THE DRAWINGS

FIGURE: shows the conserved domain within the lytic protein (LizAB48) derived from bacteriophage search result obtained by NCBI Conserved Domain Database.

### DETAILED DESCRIPTION OF THE EMBODIMENTS

The detailed description of the present invention is illustrated in the following Examples. Those skilled in the art can follow the conventional molecular biology methods in Examples and conceive the other advantages and effects of the present invention from the content described in the specification of the present invention.

### EXAMPLES

### Example 1: Culture Media and Growth Conditions

The bacteria were cultured in Luria Bertani (LB) (trypton, 10 g/L; yeast extract, 5 g/L; NaCl, 10 g/L) broth or on LB agar plates and incubated at 37° C. Phage propagation with host strains in liquid media were incubated at 30° C. For phage isolation, double plaque assay method was used. Double plaque assay was performed with two LB agar mediums with different concentrations: LB medium with 1.5% or 0.7% (w/v) agar was used for the standard agar (top layer) and for soft agar (bottom layer), respectively. Bacteriophage enrichment assays were carried out with 10× strength LB. LB broth was used for the storage of bacteria with 20% glycerol at −20° C. Bacterial growth was measured by optical density at 565 nm by densitometer turbidity detector (DEN-1, Biosan) where the bacterial cell concentration of 3×10⁸ cells/ml was calculated approximately equal to the 1 McFarland Standard Unit.

### Example 2: Bacterial Strains

All of the strains of Acinetobacter baumannii and other clinical isolates were taken from a local hospital (Ibn-i Sina Hospital, Ankara University, Turkey) isolated from various samples of hospitalized patients (ie., blood, sputum, wound etc.) in between 2015 and 2018. The isolates used in this study are Acinetobacter baumannii (n=123), other Acinetobacter species; Acinetobacter nosocomialis (n=5), Acinetobacter junii (n=5), Acinetobacter pittii (n=5). For host range analysis of the bacteriophage and its lytic enzyme, other Gram-negative bacteria; Klebsiella pneumoniae (n=5) Pseudomonas aeroginosa (n=5) Escherichia coli (n=5) and Gram-positive bacteria Enterococcus faecalis (n=5), Staphylococcus aureus (n=5) were included. In addition, standard strains were used: ATCC 25923, Escherichia coli ATCC 25922, Pseudomonas aeroginosa ATCC 27853 for host range analysis.

Clinical bacterial strains identification and their antimicrobial susceptibility profiles were provided already provided by the hospital microbiology laboratory. Bacterial clinical isolates were identified by BD Phoenix (Becton Dickinson) automated systems and MALDI-TOF. Antimicrobial susceptibilities of these isolates were tested by disk diffusion method and the results were assigned following the Clinical & Laboratory Standards Institute (CLSI) guidelines. The tested antibiotics are erythromycin, gentamicin, clindamycin, penicillin, oxacillin, cefotoxitin, vancomycin, rifampin, linezolid, teicoplanin, ciprofloxacin, quinopristi/dalfopristin, chloromphenicol, tetracycline and trimethoprim-sulfamethoxazole (Clinical and Laboratory Standards Institute, 2015).

All of the clinical bacterial strains taken from the hospital are multidrug resistant while all of the clinical Acinetobacter baumanii strains (n=123) are pan-drug resistant meaning that they are resistant to all standard antibiotics tested including colistin. Acinetobacter bauamnnii strain number 48 (AB48) is the origin host of the isolated bacteriophage MikAB48 so this strain was used for further protocols like phage and its endolysin amplification and purification steps.

### Example 3: Bacteriophage Screening and Isolation

Raw sewage water was collected from a waste treatment center (Middle East Technical University, Ankara, Turkey). Phages were screened by phage enrichment procedure. After centrifugation (9000×rpm, 10 minutes, 4° C.) of the raw sewage water, the clarified suspension (45 ml) and 5 ml of 10×strength LB broth containing 10 mM MgSO4 and 10 mM CaCl2 were mixed into a 500 ml Erlenmeyer flask. Then, 0.1 ml of ten randomly chosen overnight grown Acinetobacter baumannii clinical strains were inoculated into the mixture and incubated at 30° C. with shaking at 100 rpm. After overnight incubation, 2.5 ml of chloroform was added to the flask to kill bacteria and let 30 minutes with gentle mixing for 5-6 times at room temperature. Then, centrifugation (9000 rpm, 10 minutes, 4° C.) was performed to discard cell debris and the supernatant was filtered through a 0.45 μm pore size membrane to remove bacterial cells.

Phages were isolated by double agar layer method. Briefly, 0.1 ml of each overnight grown Acinetobacter baumannii strain was mixed with 1.5 ml of the filtered phage supernatant and 2.5 ml of melted soft agar (0.7% w/v agar). The mixture was poured evenly onto the bottom agar (1.5% w/v agar). The plates were incubated overnight at 37° C. and the cleared zones were observed on the plates where phage lysis occurs. Plates in which bacteriophages shown with clear zones were chosen for single plaque isolation. Using sterile pipette tip, the top layer with clear zone was picked up and inoculated into 2 ml LB with 0.1 ml of corresponding host Acinetobacter baumannii strain and incubated for 6 hours to enrich the potential phages. The 2 mL enriched phage filtrate was tenfold serially diluted (10⁻¹ to 10⁻⁹) in 1 ml of LB broth. 0.1 ml of overnight grown Acinetobacter baumannii host strain to each dilution was mixed with 3.5 ml of heated (60-70° C.) soft agar (0.7% w/v agar) medium and poured onto bottom agar plate (1.5% w/v agar). The plates were incubated at 37° C. overnight and checked for separated plaques on the dilution series. The well-separated single plaque was taken with a pipette tip and suspended in LB medium and again serially diluted (10¹ to 10⁻⁴). This single plaque isolation procedure was repeated three times to ensure single phage isolation.

The purified phages are named as MikAB48 according to their host strain number. MikAB48 have plaques with a halo around the plaques. Clear plaques were observed wherever phage lysate was spotted onto LB agar plates covered with a bacterial lawn of AB48. The plaque size is around 6-7 mm in diameter.

### Example 4: Phage Propagation and Concentration

The bacteriophages isolated with single plaque isolation method were propagated with their hosts for high titer phage concentration. Briefly, the single plaques on the plates were picked up from the plates with pipette tips and inoculated into a 500 ml Erlenmeyer flask with its host of Acinetobacter baumannii strain (0.5 ml overnight culture) in 50 ml LB broth. The mixture was incubated overnight at 30° C. with 100 rpm shaking. 2.5 ml of chloroform was added to lyse the bacteria in the mixture and waited 30 min with shaking for 5-6 times. After centrifigutation (9000×rpm, 10 minutes, 4° C.), the phage supernatant was filterized through a 0.45 um pore size membrane. To precipitate phages, 6 ml of sterilized solution (10% (w/v) polyethylene glycol PEG 6000 and 1M NaCl) was added to the phage filtrate and incubated overnight at 4° C. After centrifugation (15000×rpm, 20 minutes, 4° C.), the supernatant was discarded and the pellet containing the phages was resuspended in 1 ml Saline-Magnesium (SM) buffer (50 mM Tris-HCl (pH 7.5), 100 mM NaCl and 8.1 mM MgSO₄) and stored at 4° C. Purified phage lysate was supplemented with 20% glycerol and stored at −20° C. for longer term.

### Example 5: Phage Titer Calculation

The titer of concentrated single phage solution in SM buffer was determined. Tenfold dilutions of concentrated phage preparation (10⁻¹ to 10⁻¹¹) were prepared. 100 μl of the selected dilution was poured into 3 ml of soft agar medium and 100 μl of overnight Acinetobacter buamnnii culture were mixed into a test tube. The top agar mixture was poured onto a bottom standard agar plate, allowed to cool down for hardening, and then incubated overnight at 37° C. The plates with 30-300 plaques were counted. The titer of the original phage lysate was calculated as following formula: Plaque forming units/ml (pfu/ml)=(Number of plaques)×10×(1/dilution).

### Example 6: Phage Host Range Analysis (Spot Test)

The host range of isolated phages was checked by spot test. The top agar medium mixture (LB with 0.7% w/v agar, 10 mM CaCl₂ and 10 mM MgSO₄) heated in test tubes on heat block to 60-70° C. After cooling to around 50° C., 0.1 ml overnight grown tested bacteria were added into top agar medium and poured onto bottom agar plates. 10 μl of bacteriophage MikAB48 (around 3×10⁸ pfu/ml per spot) was spotted onto the bacterial lawn using a new sterile pipette for each spot and the plates were incubated overnight at 37° C.

Among the 123 clinical pan-drug resistant 46 (37.39%) were lysed by the MikAB48 while the rest of the clinical strains and standard strains (as mentioned in Example 2) are insensitive to the phage except two of the clinical Pseudomonas aeruginosa strains.

### Example 7: Phage Genomic DNA Isolation

Phage DNA was extracted from the isolated phage stock solution (3×10¹¹ pfu/ml). 100 μl of phage stock solution in SM buffer was taken into Eppendorf tube and 2 μg/μl of DNase I (Promega) was added and incubated at 37° C. for 45 minutes on heating block. Then, 2 u/μ1 DNase Stop Solution (Promega™) was added and followed by incubation on heating block at 65 C.° for 10 minutes. Proteinase K (50 μg/ml) (Macherey Nagel™) was added. Subsequently, phage MikAB48 genome isolation was performed with DNA isolation kit (DNA, RNA, and Protein Purification Kit, NucleoSpin Tissue™, Macherey-Nagel™) following the manufacturer's instructions. The eluted DNA was stored at −20° C. for further experiments.

### Example 8: Whole Genome Sequencing and Bioinformatics Analysis

The isolated bacteriophage genome concentration was measured with spectrophotometer (NanoDrop™, Thermofisher) and the DNA concentration of phage MikAB48 was 30 ng/ml. Phage DNA was sequenced at a commercial local firm. For next generation sequencing, the DNA library was constructed with Nextera sample prep kit (Illumina). Paired-end sequencing was performed by MiSeq PE300 sequencer (Illumina) with the 300 nucleotide read length.

Nucleotide sequence of the whole genome of bacteriophage MikAB48 is represented by SEQ ID NO:1

Homology of the nucleotide sequences of the isolated bacteriophage with the known bacteriophage genes was analyzed by using BLAST (https://blast.ncbi.nlm.nih.gov/Blast.cgi). According to BLAST analysis the isolated phage was named, vB_AbaA_MikAB48 (shortly MikAB48, throughout the invention) following the guidelines of International Committee on Taxonomy of Viruses (ICTV) classification since the phage belongs to order of Caudovirales and family of Ackermannviridae based on genomic sequence. The phage MikAB48 genome size is a 135242 bp, of linear, double-stranded DNA with a G+C content of 39.1%. According to BLAST analysis, the nucleotide sequence of the bacteriophage MikAB48 was confirmed to have the highest homology with the bacteriophage Acinetobacter phage SH-Ab 15599. Multiple sequence aligment results showed that MikAB48 and Acinetobacter phage SH-AB 15599 have 46% percent identity created by Clustal Omega (Clustal 12.1). To understand genetic functions of each part of the genome, ORF (Open Reading Frame) analysis was performed by GeneMarkS and functions are assigned by BLASTp.

As a result, gene sequence of the lytic protein of bacteriophage MikAB48 was obtained. The lytic protein derived from bacteriophage MikAB48 was named as LizAB48. The nucleotide sequence of the gene coding for the lytic protein derived from bacteriophage MikAB48 is represented by SEQ ID NO: 2. The amino acid sequence of the lytic protein derived from bacteriophage MikAB48 is represented by SEQ ID NO: 3.

According to the Conserved Domain Database of NCBI, LizAB48 lytic protein has a domain of Gene 25-like lysozyme superfamily (FIGURE). BLASTp of the LizAB48 showed it thas the highest homology with and lysozyme family protein Acinetobacter phage SH-Ab 1559 listed on NCBI database. Comparison of the amino acid sequences of LizAB48 and lysozyme family protein Acinetobacter phage SH-Ab 1559 by Clustal Omega (Clustal12.1) showed 68.29% identity shared.

### Example 9: Cloning of Lytic Protein Gene

From the sequencing and ORF analysis performed in Example 8, gene sequence of the lytic protein LizAB48 identified as SEQ ID NO: 2. To express the target lytic protein gene, a small scale expression system of lytic protein was constructed using pET28a vector. The gene of the lytic protein was subcloned into BamHI and Xhol restriction enzyme sites of the vector according to the conventional method. The constructed lytic protein plasmid was named pET28a-LizAB48. E. coli BL21 (DE3) was tranformed with the lytic protein expression plasmid, resulting in the preparation of a producing strain of a lytic protein.

### Example 10: Over-Expression and Purification of Lytic Protein

E. coli BL21 (DE3) cells harboring the recombinant plasmid pET28a-LizAB48 were grown overnight in LB medium containing kanamycin (50 μg/ml). An overnight culture of cells were inoculated into 300 ml of LB medium and then incubated at 37° C. until reached 0.5 of McFarland unit. To induce expression of the target protein, a final concentration of 1 mM isopropyl (3-D-1-thiogalactopyranoside (IPTG) was added to the culture followed by incubation at 20° C. for overnight. Cells were harvested by centrifugation and resuspended in 30 ml of Hepes/KOH buffer (pH 7.4, 20 mM Hepes/KOH, 140 mM NaCl, 1% Triton X-100). The cells were lysed by adding 100 μl of 1% SDS solution to the cell precipitate.

Subsequently, overproduced protein was purified. Bacteria lysate was loaded onto Ni-nitrilotriacetic acid column (Ni-NTA; Qiagen, Germany) according to the manufacturer's procedure. Proteins bound to column were eluted with with increasing imidazole concentrations (20 mM, 50 mM, 100 mM, 200 mM, 500 mM) in buffer (0.5 M NaCl, 20 mM Tris-HCl, pH 7.4). A Bradford assay was used to determine the protein concentration.

### Example 11: Antibacterial Activity of the Lytic Protein

Acinetobacter baumanii and several other Gram-negative bacteria were incubated 37° C. in LB medium until McFarland of 1 was reached. The bacteria were centrifuged (9000×rpm, 10 minutes, 20° C.), washed and suspended in potassium phosphate buffer with a pH of 8.0. Approximately 10⁷ of cells were mixed with a pH 100 μg of lysin protein LizAB48 dissolved in potassium phosphate buffer with a pH 8.0. In all cases, negative controls with the buffer mixed with lysin protein LizAB48 was performed. The mixtures were incubated in Eppendorf tubes 37° C. for 2 hours and appropriate dilutions were spread onto LB agar plates. Colonies were counted after an overnight incubation at 37° C. The antibacterial activity was quantified as the relative inactivation in logarithmic units (=log 10 (N₀/Nᵢ), where N₀=number of untreated cells (in the negative control) and Nᵢ=number of treated cells counted after incubation).

The results showed that lysin LizAB48 is active against Acinetobacter baumannii and some other Gram-negative pathogens (Table 1). Addition of LizAB48 lysin to 10⁷ Acinetobacter baumannii strain 48 caused a 3.59 log reduction within 2 hours. Lower but still significant LizAB48 lysin activity was observed in the case of Pseudomonas aeruginosa 45 (1.30 log reduction in viable cells). On the other hand, randomly selected clinical strains of Escherichia coli and Klebsiella pneumoniae were relatively insensitive to the LizAB48 antibacterial activity.

TABLE 1
Bactericidal activity of lytic protein
LizAB48 against several multidrug
resistant Gram-negative clinical strains

| | Viable |
|---|---|
| | bacterial log |
| Bacterial Strains | reduction |
| Acinetobacter baumannii 48 | 3.59 |
| Acinetobacter baumannii 37 | 2.70 |
| Pseudomonas aeruginosa 18 | 0.96 |
| Pseudomonas aeruginosa 45 | 1.30 |
| Escherichia coli 14 | 0.33 |
| Escherichia coli 7 | 0.27 |
| Klebsiella pneumoniae 5 | 0.01 |
| Klebsiella pneumoniae 1 | 0.12 |

1. (canceled)

2. A gene coding a lytic protein, wherein the lytic protein is derived from an isolated Acinetobacter baumannii phage comprising a genome set forth in SEQ ID NO: 1, and the gene encoding the lytic protein is set forth in SEQ ID NO: 2.

3. A lytic protein, wherein the lytic protein is derived from an isolated Acinetobacter baumannii phage comprising a genome set forth in SEQ ID NO: 1, and an amino acid sequence of the lytic protein is set forth in SEQ ID NO: 3.

4. A pharmaceutical composition for prevention and treatment of diseases caused by Acinetobacter baumannii, comprising a vector carrying the gene according to claim 2.

5. The pharmaceutical composition according to claim 4, comprising a vector carrying a gene of a lytic protein having at least 80% similarity to SEQ ID NO: 2.

6. A pharmaceutical composition for prevention and treatment of diseases caused by Acinetobacter baumannii, comprising the lytic protein according to claim 3.

7. The pharmaceutical composition according to claim 6, comprising an antibacterial peptide comprising of an amino acid sequence having at least 82% similarity to SEQ ID NO: 3.

8. A method of preventing and treating diseases caused by Acinetobacter baumannii, comprising the step of using the isolated Acinetobacter baumannii phage of claim 2 alone or in combination with other antibiotics or antimicrobial peptides.

9. The pharmaceutical composition according to claim 4, further comprising an antibiotic, a disinfectant, a medical cleaner, an antibacterial agent, an antibacterial cream, a medicinal agent, an antifouling agent, or a composition for removal of biofilm.

10. The pharmaceutical composition according to claim 5, further comprising an antibiotic, a disinfectant, a medical cleaner, an antibacterial agent, an antibacterial cream, a medicinal agent, an antifouling agent, or a composition for removal of biofilm.

11. The pharmaceutical composition according to claim 6, further comprising an antibiotic, a disinfectant, a medical cleaner, an antibacterial agent, an antibacterial cream, a medicinal agent, an antifouling agent, or a composition for removal of biofilm.

12. The pharmaceutical composition according to claim 7, further comprising an antibiotic, a disinfectant, a medical cleaner, an antibacterial agent, an antibacterial cream, a medicinal agent, an antifouling agent, or a composition for removal of biofilm.
